(19) United States
(12) Patent Application Publication
(10) Pub. No.: US 2026/0218169 A1
(43) Pub. Date: Jul. 30, 2026

(54) DOUBLE STRANDED DNA COMPOSITIONS AND RELATED METHODS

(71) Applicant: FLAGSHIP PIONEERING INNOVATIONS VII, LLC, Cambridge, MA (US)

(72) Inventors: Alexandra Rachael Sneider, Cambridge, MA (US); Jacob Rosenblum Rubens, Cambridge, MA (US); Camilo Ayala Breton, Andover, MA (US)

(21) Appl. No.: 18/863,919

(22) PCT Filed: May 12, 2023
(86) PCT No.: PCT/US2023/066950

§ 371 Date: Nov. 7, 2024

Related U.S. Application Data
(60) Provisional application No. 63/341,960, filed on May 13, 2022.

(51) Int. Cl. C12N 15/11; A61K 31/7105; A61K 31/7115; A61K 31/7125; A61K 31/713; A61K 38/16; A61K 48/00; C07K 19/00

(52) U.S. Cl. CPC C12N 15/11; A61K 31/7105; A61K 31/7115; A61K 31/7125; A61K 31/713; A61K 38/16; A61K 48/005; C07K 19/00; C12N 2310/315; C12N 2310/334; C12N 2310/531; C12N 2310/532; C12N 2830/34

(57) ABSTRACT

The disclosure provides, for example, therapeutic double stranded constructs (TDSCs). In some embodiments, the TDSCs comprise a double stranded DNA region, an upstream closed end, and a downstream closed end. In some embodiments, the TDSC comprises chemically modified nucleotides. In some embodiments, the TDSC is resistant to endonuclease digestion and/or resistant to immune sensor recognition, and supports expression of a heterologous payload encoded in the TDSC.

### RELATED APPLICATIONS

This application claims priority to U.S. Ser. No. 63/341,960, filed May 13, 2022, the entire contents of which is incorporated herein by reference.

### SEQUENCE LISTING

The instant application contains a Sequence Listing which has been submitted electronically in XML format and is hereby incorporated by reference in its entirety. Said XML copy, created on May 10, 2023, is named F2128-7002WO_SL.xml and is 183,436 bytes in size.

### BACKGROUND

There is a need for novel therapeutic modalities to address unmet medical need.

### SUMMARY OF THE INVENTION

Described herein are pharmaceutical DNA compositions, constructs, preparations, methods of using such compositions, constructs and preparations, and methods of making the same.

In one aspect, the invention features a therapeutic double stranded construct (“TDSC”).

### ENUMERATED EMBODIMENTS

1. A TDSC comprising:
a) an upstream exonuclease-resistant DNA end form;
b) a double stranded region; and
c) a downstream exonuclease-resistant DNA end form,
   wherein the TDSC comprises one or more chemically modified nucleotides.

2. A TDSC comprising:
a) an upstream DNA end form which is a closed end;
b) a double stranded region;
c) a downstream DNA end form which is a closed end,
   wherein the TDSC comprises one or more chemically modified nucleotides.

3. The TDSC of embodiment 1, wherein one or both of the upstream exonuclease-resistant DNA end form and downstream exonuclease-resistant DNA end form are open ends.

4. A TDSC comprising:
a) an upstream DNA end form (e.g., an upstream exonuclease-resistant DNA end form) comprising a Y-adaptor configuration;
b) a double stranded region; and
c) a downstream DNA end form (e.g., a downstream exonuclease-resistant DNA end form) comprising a Y-adaptor configuration,
   wherein the TDSC comprises one or more chemically modified nucleotides.

5. The TDSC of embodiment 1 or 3, wherein one or both of the upstream exonuclease-resistant DNA end form and downstream exonuclease-resistant DNA end form are blunt ends or sticky ends.

6. A TDSC comprising:
a) an upstream double stranded, blunt-ended DNA end form (e.g., an upstream exonuclease-resistant DNA end form that is double stranded and blunt-ended) comprising a phosphorothioate modification on each strand;
b) a double stranded region; and
c) a downstream double stranded, blunt-ended DNA end form (e.g., a downstream exonuclease-resistant DNA end form that is double stranded and blunt-ended) comprising a phosphorothioate modification on each strand,
   wherein optionally the TDSC further comprises one or more chemically modified nucleotides.

7. The TDSC of embodiment 1, wherein one or both of the upstream exonuclease-resistant DNA end form and downstream exonuclease-resistant DNA end form are closed ends.

8. The TDSC of any of embodiments 1-3, 5 or 7, wherein one or both of the upstream exonuclease-resistant DNA end form and downstream exonuclease-resistant DNA end form comprise a loop.

9. A TDSC comprising:
a) an upstream DNA end form (e.g., an upstream exonuclease-resistant DNA end form) which is a closed end;
b) a double stranded region;
c) a downstream DNA end form (e.g., a downstream exonuclease-resistant DNA end form) which is a closed end,
   wherein the TDSC comprises one or more chemically modified nucleotides.

10. The TDSC of any of embodiments 1-9, wherein the upstream DNA end form (e.g., upstream exonuclease-resistant DNA end form) comprises one or more chemically modified nucleotides.

11. The TDSC of any of embodiments 1-10, wherein the downstream DNA end form (e.g., downstream exonuclease-resistant DNA end form) comprises one or more chemically modified nucleotides.

12. The TDSC of any of embodiments 1-11, wherein one or more of the chemically modified nucleotides comprises a modification in the backbone, sugar, or base.

13. The TDSC of any of embodiments 1-12, wherein one or more of the chemically modified nucleotides is conjugated to a peptide or protein.

14. The TDSC of any of embodiments 1-13, wherein one or more chemically modified nucleotides comprises a chemically modified cytosine nucleotide and/or a phosphorothioate bond

15. The TDSC of any of embodiments 1-14, wherein one or more chemically modified nucleotides comprise a chemically modified cytosine nucleotide.

16. The TDSC of embodiment 15, wherein the chemically modified cytosine nucleotide has a substitution other than hydrogen at carbon 5 of the cytosine.

17. The TDSC of any of embodiments 1-16, wherein one or more of the chemically modified nucleotides comprises a phosphorothioate bond.

18. The TDSC of any of embodiments 1-17, wherein each of the first and second strands of the TDSC comprises one or more chemically modified nucleotides.

19. The TDSC of any of embodiments 1-18, wherein each of the first and second strands of the TDSC comprises one or more phosphorothioate bonds.

20. The TDSC of any of embodiments 1-19, wherein the upstream exonuclease-resistant DNA end form comprises at least 1, 2, 3, 4, 5, 6, 7, 8, 9, or 10 phosphorothioate bonds (e.g., between the 1, 2, 3, 4, 5, 6, 7, 8, 9, or 10 terminal nucleotides of the upstream exonuclease-resistant DNA end form, e.g., on the first strand, the second strand, or both of the first and second strands).

21. The TDSC of any of embodiments 1-20, wherein the upstream exonuclease-resistant DNA end form comprises at least 3 phosphorothioate bonds (e.g., between the 1, 2, 3, 4, 5, 6, 7, 8, 9, or 10 terminal nucleotides of the upstream exonuclease-resistant DNA end form, e.g., on the first strand, the second strand, or both of the first and second strands).

22. The TDSC of any of embodiments 1-20, wherein the upstream exonuclease-resistant DNA end form comprises at least 6 phosphorothioate bonds (e.g., between the 1, 2, 3, 4, 5, 6, 7, 8, 9, or 10 terminal nucleotides of the upstream exonuclease-resistant DNA end form, e.g., on the first strand, the second strand, or both of the first and second strands).

23. The TDSC of any of embodiments 1-22, wherein the downstream exonuclease-resistant DNA end form comprises at least 1, 2, 3, 4, 5, 6, 7, 8, 9, or 10 phosphorothioate bonds (e.g., between the 1, 2, 3, 4, 5, 6, 7, 8, 9, or 10 terminal nucleotides of the downstream exonuclease-resistant DNA end form, e.g., on the first strand, the second strand, or both of the first and second strands).

24. The TDSC of any of embodiments 1-23, wherein the downstream exonuclease-resistant DNA end form comprises at least 3 phosphorothioate bonds (e.g., between the 1, 2, 3, 4, 5, 6, 7, 8, 9, or 10 terminal nucleotides of the downstream exonuclease-resistant DNA end form, e.g., on the first strand, the second strand, or both of the first and second strands).

25. The TDSC of any of embodiments 1-23, wherein the downstream exonuclease-resistant DNA end form comprises at least 6 phosphorothioate bonds (e.g., between the 1, 2, 3, 4, 5, 6, 7, 8, 9, or 10 terminal nucleotides of the downstream exonuclease-resistant DNA end form, e.g., on the first strand, the second strand, or both of the first and second strands).

26. The TDSC of any of embodiments 1-20 or 23, wherein the upstream and downstream exonuclease-resistant DNA end form each comprises at least 1, 2, 3, 4, 5, 6, 7, 8, 9, or 10 phosphorothioate bonds (e.g., between the 1, 2, 3, 4, 5, 6, 7, 8, 9, or 10 terminal nucleotides of the upstream and downstream exonuclease-resistant DNA end forms, e.g., on the first strand, the second strand, or both of the first and second strands).

27. The TDSC of any of embodiments 1-21, 23, 24, or 26, wherein the upstream and downstream exonuclease-resistant DNA end form each comprises at least 3 phosphorothioate bonds (e.g., between the 1, 2, 3, 4, 5, 6, 7, 8, 9, or 10 terminal nucleotides of the upstream and downstream exonuclease-resistant DNA end forms, e.g., on the first strand, the second strand, or both of the first and second strands).

28. The TDSC of any of embodiments 1-20, 22, 23, 25, or 26, wherein the upstream and downstream exonuclease-resistant DNA end form each comprises at least 6 phosphorothioate bonds (e.g., between the 1, 2, 3, 4, 5, 6, 7, 8, 9, or 10 terminal nucleotides of the upstream and downstream exonuclease-resistant DNA end forms, e.g., on the first strand, the second strand, or both of the first and second strands).

29. The TDSC of any of embodiments 1-28, wherein one or more of the chemically modified nucleotides comprises a methyl group.

30. A TDSC comprising:
a) an upstream exonuclease-resistant DNA end form;
b) a double stranded region;
c) a downstream exonuclease-resistant DNA end form, wherein one or both of the upstream exonuclease-resistant DNA end form and the downstream exonuclease-resistant DNA end form comprises a Y-adaptor configuration.

31. The TDSC of embodiment 30, wherein the Y-adaptor is formed by cleavage with a uracil DNA glycosylase (UDG) and a DNA glycosylase-lyase endonuclease VIII (e.g., a USER enzyme mixture).

32. The TDSC of embodiment 30 or 31, wherein the Y-adaptor comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

33. The TDSC of any of embodiments 30-32, wherein every nucleotide in the Y-adaptor is a chemically-modified nucleotide (e.g., a phosphorothioate modified nucleotide, boranophosphate modified nucleotide, 5-methylcytosine modified nucleotide, 7-methylguanine modified nucleotide, and/or methylated nucleotide).

34. A TDSC comprising:
a) an upstream exonuclease-resistant DNA end form;
b) a double stranded region;
c) a downstream exonuclease-resistant DNA end form, wherein one or both of the upstream exonuclease-resistant DNA end form and the downstream exonuclease-resistant DNA end form comprises one or more of: a nuclear targeting sequence, a maintenance sequence, or a sequence that binds an endogenous polypeptide in a target cell.

35. A TDSC comprising:
a) an upstream exonuclease-resistant DNA end form;
b) a double stranded region;
c) a downstream exonuclease-resistant DNA end form, wherein one or both of the upstream exonuclease-resistant DNA end form and the downstream exonuclease-resistant DNA end form have one or more of the following characteristics:
i) does not comprise the nucleic acid sequences TATCAGCACACAATTGCCCATTATACGC (SEQ ID NO: 55) and GCGTATAATGGGCAATTGTGTGCTGATA (SEQ ID NO: 56), or nucleic acid sequences having at least 75%, 80%, 85%, 90%, 95%, 96%, 97%, 98%, or 99% sequence identity thereto; and/or the nucleic acid sequences TATCAGCACACAATAGTCCATTATACGC (SEQ ID NO: 57) and GCGTATAATGGACTATTGTGTGCTGATA (SEQ ID NO: 58);
ii) every nucleotide in the TDSC binds another nucleotide in the TDSC;
iii) the upstream exonuclease-resistant DNA end form has a loop size of less than about 28 or 56 nucleotides in length or greater than about 28 or 56 nucleotides in length; or
iv) the downstream exonuclease-resistant DNA end form has a loop size of less than about 28 or 56 nucleotides in length or greater than about 28 or 56 nucleotides in length.

36. The TDSC of any of the preceding embodiments, which comprises one or more of:
i) a promoter sequence (wherein optionally the promoter sequence is in the double stranded region);
ii) a payload sequence (e.g., a therapeutic payload sequence) operably linked to the promoter sequence (wherein optionally the payload sequence is in the double stranded region);
iii) a heterologous functional sequence, e.g., a nuclear targeting sequence or a regulatory sequence;
iv) a maintenance sequence; and/or
v) an origin of replication.

37. The TDSC of embodiment 36, which comprises:
i, ii, and iii;
i, ii, and iv;
i, ii, and v;
i, ii, iii, and iv;
i, ii, iii, and v;
i, ii, iv, and v; or
i, ii, iii, iv, and v.

38. The TDSC of embodiment 36 or 37, wherein the nuclear targeting sequence comprises a CT3 sequence (e.g., a sequence of AATTCTCCTCCCCACCTTCCCCACCCTCCCCA (SEQ ID NO: 59)), or a nucleic acid sequence having at least 75%, 80%, 85%, 90%, 95%, 96%, 97%, 98%, or 99% sequence identity thereto.

39. The TDSC of any of embodiments 36-38, wherein the nuclear targeting sequence binds to a hnRNPK protein (e.g., a human hnRNPK protein).

40. The TDSC of any of embodiments 2, 7-29, or 36-39, wherein one or both of the closed ends comprise a loop, wherein one or both of the loops comprise a nuclear targeting sequence as listed in Table 3, or a nucleic acid sequence having at least 75%, 80%, 85%, 90%, 95%, 96%, 97%, 98%, or 99% identity thereto.

41. The TDSC of any of embodiments 2, 7-29, or 36-40, wherein one or both of the closed ends comprise a loop, wherein one or both of the loops comprise a nuclear targeting sequence that binds to a nuclear import protein as listed in Table 3.

42. The TDSC of any of embodiments 36-41, wherein the payload sequence encodes a polypeptide (e.g., a protein).

43. The TDSC of any of embodiments 36-42, wherein the payload sequence encodes a functional RNA (e.g., a miRNA, siRNA, or tRNA).

44. The TDSC of any of embodiments 36-43, wherein the payload sequence is heterologous to a target cell.

45. The TDSC of any of embodiments 1-44, wherein the double stranded region comprises a sense strand and an antisense strand.

46. The TDSC of embodiment 45, wherein the antisense strand comprises one or more chemically modified nucleotides.

47. The TDSC of embodiment 45 or 46, wherein the sense strand does not comprise any chemically modified nucleotides.

48. The TDSC of embodiment 45 or 46, wherein the sense strand comprises one or more chemically modified nucleotides.

49. The TDSC of any of the preceding embodiments, wherein the TDSC is resistant to endonuclease digestion and/or resistant to immune sensor recognition.

50. The TDSC of any of the preceding embodiments, wherein the upstream exonuclease-resistant DNA end form is resistant to endonuclease digestion.

51. The TDSC of any of the preceding embodiments, wherein the upstream exonuclease-resistant DNA end form is resistant to immune sensor recognition.

52. The TDSC of any of the preceding embodiments, wherein the downstream exonuclease-resistant DNA end form is resistant to endonuclease digestion.

53. The TDSC of any of the preceding embodiments, wherein the downstream exonuclease-resistant DNA end form is resistant to immune sensor recognition.

54. The TDSC of any of the preceding embodiments, wherein the double stranded region is resistant to endonuclease digestion.

55. The TDSC of any of the preceding embodiments, wherein the double stranded region is resistant to immune sensor recognition.

56. The TDSC of any of the preceding embodiments, wherein the upstream DNA end form and the downstream DNA end form have the same nucleotide sequence.

57. The TDSC of any of embodiments 1-55, wherein the upstream DNA end form and the downstream DNA end form have different nucleotide sequences.

58. The TDSC of any of the preceding embodiments, wherein the upstream exonuclease-resistant DNA end form and the downstream exonuclease-resistant DNA end form have the same structure.

59. The TDSC of any of embodiments 1-57, wherein the upstream exonuclease-resistant DNA end form and the downstream exonuclease-resistant DNA end form have different structures.

60. The TDSC of any of embodiments 1, 7, 8, or 10-59, wherein one or both of the upstream exonuclease-resistant DNA end form and downstream exonuclease-resistant DNA end form are open ends (e.g., blunt ends, sticky ends, or Y-adaptors).

61. The TDSC of any of embodiments 1-3, 5, or 7-60, wherein one or both of the upstream exonuclease-resistant DNA end form and downstream exonuclease-resistant DNA end form are closed ends (e.g., hairpins).

62. The TDSC of embodiment 61, wherein the closed end comprises one or more (e.g., at least 1, 2, 3, 4, 5, 6, 7, 8, 9, 10, 15, 20, 25, 30, 40, or 50) nucleotides that are not hybridized (e.g., are not part of a double-stranded region).

63. The TDSC of embodiment 61, wherein the closed end does not comprise any nucleotides that are not hybridized (e.g., wherein all nucleotides of the closed end are hybridized to another nucleotide).

64. The TDSC of any of embodiments 30-63, wherein the upstream DNA end form, the downstream DNA end form, or both, comprise at least one chemically modified nucleotide.

65. The TDSC of any of embodiments 30-64, wherein both of the upstream DNA end form and the downstream DNA end form comprise at least one chemically modified nucleotide on the sense strand and at least one chemically modified nucleotide on the antisense strand.

66. The TDSC of any of embodiments 30-65, wherein both the upstream DNA end form and the downstream DNA end form comprise chemically modified nucleotides at every sense strand position and every antisense strand position.

67. The TDSC of any of embodiments 30, 31, 34-45, 47, or 49-63, wherein the upstream DNA end form, the downstream DNA end form, or both, comprises an inverted terminal repeat (ITR), wherein optionally the dsDNA comprises no chemically modified nucleotides.

68. The TDSC of any of embodiments 1-67, wherein the upstream DNA end form, the downstream DNA end form, or both, does not comprise a protelomerase sequence.

69. The TDSC of any of embodiments 30, 31, 34-45, 47, 49-63, or 67, wherein the upstream DNA end form, the downstream DNA end form, or both, comprises a protelomerase sequence, wherein optionally the dsDNA comprises no chemically modified nucleotides.

70. The TDSC of embodiment 69, wherein one or more of the protelomerase sequences comprise (e.g., in 5′-to-3′ order) the nucleic acid sequences TATCAGCACACAATTGCCCATTATACGC (SEQ ID NO: 55) and GCGTATAATGGGCAATTGTGTGCTGATA (SEQ ID NO: 56), or nucleic acid sequences having at least 75%, 80%, 85%, 90%, 95%, 96%, 97%, 98%, or 99% sequence identity thereto.

71. The TDSC of embodiment 69 or 70, wherein one or more of the protelomerase sequences comprise (e.g., in 5′-to-3′ order) the nucleic acid sequences TATCAGCACACAATAGTCCATTATACGC (SEQ ID NO: 57) and GCGTATAATGGACTATTGTGTGCTGATA (SEQ ID NO: 58), or nucleic acid sequences having at least 75%, 80%, 85%, 90%, 95%, 96%, 97%, 98%, or 99% sequence identity thereto.

72. The TDSC of any of embodiments 69-71, wherein one or more of the protelomerase sequences comprise (e.g., in 5′-to-3′ order) the nucleic acid sequences ACCTATTTCAGCATACTACGC (SEQ ID NO: 60) and GCGTAGTATGCTGAAATAGGT (SEQ ID NO: 61), or nucleic acid sequences having at least 75%, 80%, 85%, 90%, 95%, 96%, 97%, 98%, or 99% sequence identity thereto.

73. The TDSC of any of embodiments 69-72, wherein one or more of the protelomerase sequences comprise (e.g., in 5′-to-3′ order) the nucleic acid sequence CACACAATTGCCCATTATACGCGCGTATAATGGGCAATTGTGTG (SEQ ID NO: 62), or a nucleic acid sequence having at least 75%, 80%, 85%, 90%, 95%, 96%, 97%, 98%, or 99% sequence identity thereto.

74. The TDSC of any of embodiments 69-73, wherein one or more of the protelomerase sequences comprise (e.g., in 5′-to-3′ order) the nucleic acid sequences:
(i) TAAATATAATTTAA (SEQ ID NO: 63) and TTAAATTATATTTA (SEQ ID NO: 64),
(ii) AATATATAATCTAA (SEQ ID NO: 65) and TTAGATTATATATT (SEQ ID NO: 66),
(iii) TATTTATTATCTTT (SEQ ID NO: 67) and AAAGATAATAAATA (SEQ ID NO: 68),
(iv) ATATAATTTTTAATTAGTATAGAATATGTTAA (SEQ ID NO: 69) and TTAACATACTCTATACTAATTAAAAATTATAT (SEQ ID NO: 70),
(v) TATAATTTGATATTAGTACAAATCCC (SEQ ID NO: 71) and GGGATTTGTACTAATATCAAATTATA (SEQ ID NO: 72),
(vi) ATATAATATTTATTTAGTACAAAGTTC (SEQ ID NO: 73) and GAACTTTGTACTAAATAAATATTATAT (SEQ ID NO: 74),
(vii) ATATAATTTTTTATTAGTATAGAGTAT (SEQ ID NO: 75) and ATACTCTATACTAATAAAAAATTATAT (SEQ ID NO: 76),
(viii) TAAATATAATTTAA (SEQ ID NO: 63) and TTAAATTATATTTA (SEQ ID NO: 64); or nucleic acid sequences having at least 75%, 80%, 85%, 90%, 95%, 96%, 97%, 98%, or 99% sequence identity thereto.

75. The TDSC of any of embodiments 69-74, wherein one or more of the protelomerase sequences further comprise (e.g., in 5′-to-3′ order) the nucleic acid sequences:
(i) TAGTATAAAAAACTGT (SEQ ID NO: 77) and ACAGTTTTTTATACTA (SEQ ID NO: 78),
(ii) TAGTATACAAAAGATT (SEQ ID NO: 79) and AATCTTTTGTATACTA (SEQ ID NO: 80),
(iii) TAGTATATATATCTCT (SEQ ID NO: 81) and AGAGATATATATACTA (SEQ ID NO: 82), or
(viii) TAGTATAAAAAAAATT (SEQ ID NO: 83) and AATTTTTTTTATACTA (SEQ ID NO: 84);
or nucleic acid sequences having at least 75%, 80%, 85%, 90%, 95%, 96%, 97%, 98%, or 99% sequence identity thereto.

76. The TDSC of any of embodiments 69-75, wherein the protelomerase sequences are produced by TelN protelomerase, ResT protelomerase, Tel PY54 protelomerase, or TelK protelomerase digestion.

77. The TDSC of any of embodiments 69-75, wherein the protelomerase sequences are not produced by TelN protelomerase digestion.

78. The TDSC of any of embodiments 69-75 or 77, wherein the protelomerase sequences are not produced by Tel PY54 protelomerase digestion.

79. The TDSC of any of embodiments 69-75, 77, or 78, wherein the protelomerase sequences are not produced by TelK protelomerase digestion.

80. The TDSC of any of embodiments 69-75 or 77-79, wherein the protelomerase sequences are not produced by ResT protelomerase digestion.

81. The TDSC of any of embodiments 69-80, wherein the protelomerase sequences are about 28 or 56 nucleotides in length.

82. The TDSC of any of embodiments 69-81, wherein the protelomerase sequences are less than 28 (e.g., less than 15, 20, 25, 26, 27, or 28) nucleotides in length.

83. The TDSC of any of embodiments 69-82, wherein the protelomerase sequences are between about 28 (e.g., 25, 26, 27, 28, 29, 30, 31, 32, 33, 34, or 35) nucleotides and about 56 (e.g., 50, 51, 52, 53, 54, 55, 56, 57, 58, 59, or 60) nucleotides in length.

84. The TDSC of any of embodiments 69-83, wherein the protelomerase sequences are greater than about 56 (e.g., greater than 50, 51, 52, 53, 54, 55, 56, 57, 58, 59, 60, 65, 70, 75, 80, 90, or 100) nucleotides in length.

85. The TDSC of any of embodiments 30-94, wherein the upstream DNA end form, the downstream DNA end form, or both, comprises a Y-adaptor.

86. The TDSC of embodiment 85, wherein optionally the dsDNA comprises no chemically modified nucleotides.

87. The TDSC of embodiment 69, wherein the protelomerase sequence is produced from a first protelomerase recognition sequence (PRS) and a second PRS that are recognized by a TelN protelomerase or ResT protelomerase.

88. The TDSC of embodiment 69, wherein the protelomerase sequence is produced from a first protelomerase recognition sequence (PRS) and a second PRS that are recognized by a Tel PY54 protelomerase or TelK protelomerase.

89. The TDSC of any of embodiments 1-85, 87, or 88, wherein one or both of the upstream exonuclease-resistant DNA end form and the downstream exonuclease-resistant DNA end form comprises at least one chemically modified nucleotide (e.g., comprises a chemical modification on every sense strand nucleotide and every antisense strand nucleotide).

90. The TDSC of any of embodiments 1-85 or 87-89, wherein one or both of the upstream exonuclease-resistant DNA end form and the downstream exonuclease-resistant DNA end form comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

91. The TDSC of any of embodiments 1-85 or 87-90, wherein the double-stranded region comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

92. The TDSC of any of embodiments 1-85 or 87-91, wherein the double-stranded region encodes a payload sequence, and wherein the antisense strand for the payload sequence comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

93. The TDSC of any of embodiments 1-92, wherein the double-stranded region encodes a payload sequence, and wherein the sense strand for the payload sequence comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

94. The TDSC of any of the preceding embodiments, wherein 80%, 85%, 90%, 95%, 98%, 99%, or 100% of the sugars of the TDSC are deoxyribose sugars.

95. The TDSC of any of the preceding embodiments, wherein the TDSC comprises a sequence encoding an RNA (e.g., an mRNA, siRNA, or miRNA).

96. The TDSC of any of embodiments 1-94, wherein the TDSC does not comprise a sequence encoding an RNA.

97. The TDSC of any of the preceding embodiments, wherein the TDSC can be replicated (e.g., by a DNA polymerase native to a cell comprising the TDSC).

98. The TDSC of any of embodiments 1-96, wherein the TDSC cannot be replicated.

99. The TDSC of any of the preceding embodiments, wherein the TDSC is linear and can be circularized.

100. The TDSC of any of embodiments 1-98, wherein the TDSC is linear and cannot be circularized.

101. The TDSC of any of the preceding embodiments, wherein the TDSC or a portion thereof can be integrated into the genome.

102. The TDSC of any of embodiments 1-100, wherein the TDSC or a portion thereof cannot be integrated into the genome. 103. The TDSC of any of the preceding embodiments, wherein the TDSC can be concatemerized.

104. The TDSC of any of embodiments 1-102, wherein the TDSC cannot be concatemerized.

105. A pharmaceutical composition comprising a double stranded DNA (dsDNA) comprising an effector sequence, wherein:
a. the dsDNA lacks a vector backbone or lacks a material portion of vector backbone, or does not comprise a non-human (e.g., bacterial) origin of replication;
b. the dsDNA is unencapsidated, is essentially free of viral proteins, does not comprise a viral packaging signal, or does not comprise a viral ITR;
c. the dsDNA comprises exonuclease-resistant ends; and
d. the dsDNA comprises at least one chemically modified nucleotide.

106. A pharmaceutical composition comprising a TDSC of any of the preceding embodiments.

107. The pharmaceutical composition of embodiment 105 or 106, wherein the dsDNA or TDSC is comprised in a lipid nanoparticle (LNP).

108. The pharmaceutical composition of any of embodiments 105-107, further comprising an electroporation buffer.

109. The pharmaceutical composition of any of embodiments 105-108, further comprising a transfection reagent.

110. A proto-TDSC comprising:
a) an upstream exonuclease-resistant DNA end form;
b) a double stranded region;
c) a downstream exonuclease-resistant DNA end form,
   wherein the proto-TDSC comprises one or more (e.g., 1 or 2) uracil nucleotides.

111. The proto-TDSC of embodiment 110, wherein the upstream exonuclease-resistant DNA end form comprises one or more (e.g., 1 or 2) uracil nucleotides.

112. The proto-TDSC of embodiment 110 or 111, wherein the downstream exonuclease-resistant DNA end form comprises one or more (e.g., 1 or 2) uracil nucleotides.

113. The proto-TDSC of any of embodiments 110-112, wherein the upstream exonuclease-resistant DNA end form comprises a loop structure.

114. The proto-TDSC of any of embodiments 110-113, wherein the downstream exonuclease-resistant DNA end form comprises a loop structure.

115. The proto-TDSC of any of embodiments 110-114, wherein the upstream exonuclease-resistant DNA end form comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

116. The proto-TDSC of any of embodiments 110-115, wherein every nucleotide in the upstream exonuclease-resistant DNA end form is a chemically-modified nucleotide (e.g., a phosphorothioate modified nucleotides, boranophosphate modified nucleotides, 5-methylcytosine modified nucleotides, 7-methylguanine modified nucleotides, and/or methylated nucleotides).

117. The proto-TDSC of any of embodiments 110-116, wherein the downstream exonuclease-resistant DNA end form comprises one or more chemically-modified nucleotides (e.g., phosphorothioate modified nucleotides, boranophosphate modified nucleotides, 5-methylcytosine modified nucleotides, 7-methylguanine modified nucleotides, and/or methylated nucleotides).

118. The proto-TDSC of any of embodiments 110-117, wherein every nucleotide in the downstream exonuclease-resistant DNA end form is a chemically-modified nucleotide (e.g., a phosphorothioate modified nucleotides, boranophosphate modified nucleotides, 5-methylcytosine modified nucleotides, 7-methylguanine modified nucleotides, and/or methylated nucleotides).

119. A proto-TDSC comprising:
a) an upstream exonuclease-resistant DNA end form;
b) a double stranded region;
c) a downstream exonuclease-resistant DNA end form,
   wherein the upstream exonuclease-resistant DNA end form comprises a sticky end, and/or
   wherein the downstream exonuclease-resistant DNA end form comprises a sticky end.

120. A method of expressing a heterologous payload in a target cell, the method comprising:
(i) introducing into a target cell a TDSC or composition of any of the preceding embodiments, wherein the double-stranded region of the TDSC comprises a sequence encoding a heterologous payload; and
(ii) maintaining (e.g., incubating) the cell under conditions suitable for expressing the heterologous payload from the TDSC;
thereby expressing the heterologous payload in the target cell.

121. A method of expressing a heterologous payload in a target cell, the method comprising:
(i) providing a target cell comprising a TDSC or composition of any of embodiments 1-119, wherein the double-stranded region of the TDSC comprises a sequence encoding a heterologous payload; and
(ii) maintaining (e.g., incubating) the cell under conditions suitable for expressing the heterologous payload from the TDSC;
thereby expressing the heterologous payload in the target cell.

122. The method of embodiment 120 or 121, which is performed ex vivo or in vivo.

123. A method of delivering a heterologous payload to a target cell, the method comprising:
introducing into a target cell a TDSC or composition of any of embodiments 1-119, wherein the double-stranded region of the TDSC comprises a sequence encoding a heterologous payload;
thereby delivering the heterologous payload to the target cell.

124. A method of modulating (e.g., increasing or decreasing) a biological activity in a target cell, the method comprising:
(i) introducing into a target cell a TDSC or composition of any of embodiments 1-119, wherein the double-stranded region of the TDSC comprises a sequence encoding a heterologous payload that modulates a biological activity in the target cell; and
(ii) maintaining (e.g., incubating) the cell under conditions suitable for expressing the heterologous payload from the TDSC;
thereby modulating the biological activity in the target cell.

125. A method of modulating (e.g., increasing or decreasing) a biological activity in a target cell, the method comprising:
(i) providing a target cell comprising a TDSC or composition of any of embodiments 1-119, wherein the double-stranded region of the TDSC comprises a sequence encoding a heterologous payload that modulates a biological activity in the target cell; and
(ii) maintaining (e.g., incubating) the cell under conditions suitable for expressing the heterologous payload from the TDSC;
thereby modulating the biological activity in the target cell.

126. The method of embodiment 124 or 125, wherein the heterologous payload increases the biological activity in the target cell.

127. The method of embodiment 124 or 125, wherein the heterologous payload decreases the biological activity in the target cell.

128. The method of any of embodiments 124-127, wherein the biological activity comprises cell growth, cell metabolism, cell signaling, cell movement, specialization, interactions, division, transport, homeostasis, osmosis, or diffusion.

129. The method of any of embodiments 120-128, wherein the cell is an animal cell, e.g., a mammalian cell, e.g., a human cell.

130. A method of treating a cell, tissue, or subject in need thereof, the method comprising:
administering to the cell, tissue, or subject a TDSC or composition of any of embodiments 1-119, wherein the double-stranded region of the TDSC comprises a sequence encoding a heterologous payload;
thereby treating the cell, tissue, or subject.

131. A method of making a TDSC, the method comprising:
(i) ligating:
   a double-stranded DNA molecule to
   a hairpin DNA molecule comprising: a loop region and a double-stranded region comprising one or more chemically modified nucleotides;
   thereby producing a ligated dsDNA; and
(ii) incubating the ligated dsDNA with an enzyme that cleaves the loop region from the ligated dsDNA;
thereby making a TDSC.

132. The method of embodiment 131, further comprising incubating the dsDNA (e.g., after step ii) with a blunt end-producing enzyme (e.g., a Mung bean nuclease).

133. The method of embodiment 131 or 132, wherein the enzyme that cleaves the loop region from the ligated dsDNA is a uracil DNA glycosylase (UDG) and a DNA glycosylase-lyase endonuclease VIII (e.g., a USER enzyme mixture).

134. A method of making a TDSC, the method comprising:
(i) ligating:
   a double-stranded DNA molecule to
   a hairpin DNA molecule comprising:
      a loop region, and
      a double-stranded region,
      wherein the hairpin DNA molecule comprises one or more chemically modified nucleotides, e.g., in the loop region;
   thereby producing a ligated dsDNA; and
(ii) incubating the ligated dsDNA with an enzyme that opens or cleaves the loop region; thereby making a TDSC.

135. The method of embodiment 134, wherein the loop region comprises a uracil nucleotide.

136. The method of embodiment 134 or 135, wherein the enzyme that opens or cleaves the loop region is a uracil DNA glycosylase (UDG) and a DNA glycosylase-lyase endonuclease VIII (e.g., a USER enzyme mixture).

137. A method of making a TDSC, the method comprising ligating:
a double-stranded DNA molecule to
a self-annealed DNA molecule comprising a first region and a second region, wherein the first region is hybridized to the second region;
thereby producing a TDSC.

138. The method of embodiment 137, wherein the self-annealed DNA molecule further comprises a loop between the first region and the second region.

139. The method of embodiment 138, wherein the loop comprises a heterologous functional sequence, e.g., a nuclear targeting sequence (e.g., a CT3 sequence); or a regulatory sequence.

140. The method of embodiment 138 or 139, wherein the loop comprises a nuclear targeting sequence as listed in Table 3, or a nucleic acid sequence having at least 75%, 80%, 85%, 90%, 95%, 96%, 97%, 98%, or 99% identity thereto.

141. The method of any of embodiments 138-140, wherein the loop comprises a nuclear targeting sequence that binds to a nuclear import protein as listed in Table 3.

142. The method of any of embodiments 137-141, wherein the self-annealed DNA molecule does not comprise any nucleotides that are not hybridized (e.g., wherein all nucleotides of the self-annealed DNA molecule are hybridized to another nucleotide).

143. The method of any of embodiments 131-142, which further comprises ligating a second hairpin DNA molecule to the double-stranded DNA molecule, wherein the second hairpin DNA molecule comprises a loop region and a double-stranded region, wherein optionally the second hairpin DNA molecule comprises one or more chemically modified nucleotides in one or both of the loop region or the double stranded region.

144. A method of making or manufacturing a TDSC, the method comprising:
a) providing a TDSC comprising closed ends, e.g., a TDSC described herein;
b) incubating the TDSC with a double stranded DNA exonuclease, e.g., Exonuclease III, e.g., e.g., 1 μL of Exonuclease III per 5 μg of DNA in 50 μL, for 1 hour at 37° C., e.g., as described in Example 10;
c) optionally, purifying the TDSC treated in step b), e.g., by Silica membrane column, e.g., as described in Example 10,
thereby making or manufacturing the TDSC.

145. A method of making or manufacturing a TDSC, the method comprising:
a) providing a proto-TDSC, e.g., a proto-TDSC that has been treated with Exonuclease III, wherein the proto-TDSC comprises closed DNA end forms each comprising a uracil;
b) incubating the proto-TDSC with a uracil excision enzyme, e.g., USER enzyme, e.g., 3 μL of USER enzyme is to 5 μg of DNA in 100 μL, e.g., for 1 hour at 37° C., e.g., as described in Example 12;
c) optionally, incubating the TDSC with a single strand DNA nuclease, e.g., mung bean nuclease, e.g., 10 U of mung bean nuclease to 5 μg of DNA in about 100 μL, e.g., for 30 min at 30° C., e.g., as described in Example 12;
d) optionally, purifying the TDSC treated in step c), e.g., by Silica membrane column, e.g., as described in Example 12,
thereby making or manufacturing the TDSC.

146. The method of embodiment 144 or 145, which further comprises assaying the TDSC for degradation, e.g., by agarose gel, e.g., as described in Example 10.

147. The method of embodiment 146, which further comprises, responsive to the assay for degradation (e.g., responsive to a determination that degradation is below a predetermined value), performing one of more of: releasing the TDSC, placing the TDSC into a container, formulating the TDSC, or adding one or more excipients to the TDSC.

148. A method of making or manufacturing a TDSC, the method comprising:
a) providing a TDSC, e.g., a TDSC of any of embodiments 1-119;
b) determining whether the structure of the TDSC matches a reference structure;
thereby making or manufacturing the TDSC.

149. The method of embodiment 148, wherein the determining of (b) comprises sequencing the TDSC.

150. The method of embodiment 148 or 149, wherein the determining of (b) comprises digesting the TDSC with a restriction enzyme.

151. The method of any of embodiments 148-150, wherein the structure of the TDSC that matches the reference structure is identical to the reference structure.

152. The method of any of embodiments 148-151, wherein the structure of the TDSC that matches the reference structure has the same sequence as the reference structure.

153. The method of any of embodiments 148-152, wherein the structure of the TDSC that matches the reference structure has the same length as the reference structure.

In an embodiment, the TDSC has at least 15 nucleotides, at least 30 nucleotides, at least 50 nucleotides, at least 75 nucleotides, 100 nucleotides, at least 200 nucleotides, at least 300 nucleotides, at least 500 nucleotides, at least 750 nucleotides, at least 1,000 nucleotides, at least 2,000 nucleotides, at least 3,000 nucleotides, at least 4,000 nucleotides, at least 5,000 nucleotides, at least 10,000 nucleotides, at least 15,000 nucleotides, at least 20,000 nucleotides, at least 25,000 nucleotides, at least 30,000 nucleotides, at least 35,000 nucleotides, at least 40,000 nucleotides at least 45,000 nucleotides, at least 50,000 nucleotides, at least 60,000 nucleotides, or more.

In an embodiment, the TDSC has between 20 and 1000 nucleotides, between 20 and 50 nucleotides, between 100 and 500 nucleotides, between 500 and 50,000 nucleotides, between 1,000 and 50,000 nucleotides, between 2,000 and 40,000 nucleotides, between 5,000 and 50,000 nucleotides, between 500 and 50,000 nucleotides, between 500 and 25,000 nucleotides, between 1,000 and 20,000 nucleotides, between 1,000 and 10,000 nucleotides, between 10,000 and 60,000 nucleotides, between 1,000 and 20,000 nucleotides, between 1,000 and 40,000 nucleotides.

In an embodiment, the TDSC comprises at least one nucleotide modification, e.g., a covalent nucleotide modification, e.g., selected from: N6-Methyladenosine (m6A, 6 mA); 5-formylcytosine (5-formyl-2′-deoxycytosine, 5fC, f5C); 5-carboxylcytosine (5-carboxyl-2′-deoxycytosine, 5-carboxycytosine, ca5C, 5caC); 5-hydroxymethylcytosine (5-hydroxymethyl-2′-deoxycytosine, 5hmC, hm5C); 5-methyldeoxycytosine (5-methylcytosine; 5-methyl-2′-deoxycytosine; m5dC; 5mC, m5C); 5′-methylcytosine; 3-methylcytosine (m3C); 5-methyl pyrimidine; 8-oxoguanine (8-oxoG); phosphorothioate; S and R phsophorothioate linkages; methylthymine; N3′-P5′ Phosphoroamidate (NP). In some embodiments, the nucleotide modification is a base modification. In some embodiments, the nucleotide modification is a backbone modification. In some embodiments, the nucleotide modification is a sugar modification. In some embodiments, the nucleotide modification comprises a peptide conjugate. In some embodiments, the nucleotide modification comprises a protein conjugate.

In an embodiment, the effector sequence is a DNA sequence encoding a therapeutic RNA (e.g., mRNA or regulatory RNA), operably linked to a promoter. In an embodiment, the RNA can be, e.g., an mRNA, a tRNA, lncRNA, miRNA, IRNA, snRNA, microRNA, siRNA, piRNA, snoRNA, snRNA, exRNA, scaRNA, Y RNA, or hnRNA.

In an embodiment, the effector sequence is a DNA sequence encoding a therapeutic peptide or polypeptide, operably linked to a promoter. The therapeutic peptide or polypeptide may be, e.g., a DNA binding protein; an RNA binding protein; a transporter; a transcription factor; a translation factor; a ribosomal protein; a chromatin remodeling factor; an epigenetic modifying factor; an antigen; a hormone; an enzyme (such as a nuclease, e.g., an endonuclease, e.g., a nuclease element of a CRISPR system, e.g., a Cas9, dCas9, a Cas9-nickase, Cpf/Cas12a); a Crispr-linked enzyme, e.g., a base editor or prime editor; a mobile genetic element protein (e.g., a transposase, a retrotransposase, a recombinase, an integrase); a Gene Writer polypeptide; a polymerase; a methylase; a demethylase; an acetylase; a deacetylase; a kinase; a phosphatase; a ligase; a deubiquitinase; a protease; an integrase; a recombinase; a topoisomerase; a gyrase; a helicase; a lysosomal acid hydrolase); an antibody (e.g., an intact antibody, a fragment thereof, or a nanobody); a signaling peptide; a receptor ligand; a receptor; a clotting factor; a coagulation factor; a structural protein; a caspase; a membrane protein; a mitochondrial protein; a nuclear protein; or an engineered binder such as a centyrin, darpin, or adnectin. In an embodiment, the effector sequence is a DNA sequence encoding a reporter protein.

In embodiments, the TDSC can include a plurality of effector sequences. The plurality may be the same or different types, e.g., a TDSC can include an effector sequence that is a structural DNA and a second effector sequence that is a DNA sequence encoding a functional RNA or polypeptide. A TDSC can include an effector sequence that is a DNA sequence encoding a functional RNA and a second effector sequence that is a DNA sequence encoding a functional polypeptide. The plurality of effector sequences may be the same or different sequences of the same type.

In embodiments, the TDSC is not disposed in a carrier, e.g., it is formulated for naked administration.

In embodiments, the TDSC is formulated with a carrier, e.g., a lipid-based carrier, e.g., an LNP.

In embodiments, the TDSC is formulated with a pharmaceutical excipient.

In embodiments, the TDSC is formulated for parenteral administration.

In embodiments, the pharmaceutical composition is formulated for topical administration.

In embodiments, the pharmaceutical composition is substantially free of impurities or process byproducts, e.g., selected from the group consisting of: endotoxin, mononucleotides, chemically modified mononucleotides, DNA fragments or truncations, and proteins (e.g., enzymes, e.g., ligases, restriction enzymes). In some embodiments, the pharmaceutical composition is substantially free of circular DNA.

In another aspect, the invention includes a method of delivering an effector to a subject, e.g., a subject in need thereof. The method incudes administering to the subject a composition described herein, e.g., described in any embodiment above. In an embodiment, the subject has or has been diagnosed with a condition that can be treated with the effector.

In another aspect, the invention includes a method of modulating (e.g., increasing or decreasing) a biological parameter in a cell, tissue or subject. The method incudes administering to the subject a composition described herein, e.g., described in any embodiment above. In embodiments, the biological parameter is an increase or decrease in gene expression of a subject gene in a target cell, tissue or subject, which increase or decrease is effected by an effector sequence described herein. In an embodiment, the subject has or has been diagnosed with a condition that can be treated with the effector.

In another aspect, the invention includes a method of treating a cell, tissue or subject. The method includes administering to a cell, tissue or subject in need thereof an TDSC or construct described herein, e.g., described in any embodiment above. In an embodiment, the subject has or has been diagnosed with a condition that can be treated with the effector.

The disclosure also provides method of making the TDSCs and dsDNA compositions described herein. In an embodiment, the method comprises performing golden gate assembly.

In an embodiment, the method further comprises enriching or purifying the TDSC.

In an embodiment, the enriching or purifying includes substantially removing from the TDSC one or more impurity selected from: endotoxin, mononucleotides, chemically modified mononucleotides, single stranded DNA, DNA fragments or truncations, and proteins (e.g., enzymes, e.g., ligases, restriction enzymes).

In an embodiment, the method further comprises formulating the enriched or purified TDSC for pharmaceutical use, e.g., formulating the TDSC with a pharmaceutically acceptable excipient and/or with a carrier, e.g., an LNP.

### Definitions

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi-tri- and quad-specific antibodies, diabodies, triabodies, and tetrabodies), single-domain antibodies (sdAb), epitope-binding fragments, e.g., Fab, Fab′ and F(ab′).sub.2, Fd, Fvs, single-chain Fvs (scFv), rlgG, single-chain antibodies, disulfide-linked Fvs (sdFv), nanobody, fragments including either a VL or VH domain, fragments produced by an Fab expression library, and anti-idiotypic (anti-Id) antibodies. Antibodies described herein can be of any type (e.g., IgG, IgE, IgM, IgD, IgA, and IgY), class (e.g., IgG1, IgG2, IgG3, IgG4, IgAQ1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)2 fragments) that are capable of specifically binding to a target protein. Fab and F(ab′)2 fragments lack the Fc fragment of an intact antibody.

As used herein, the term “carrier” means a compound, composition, reagent, or molecule that facilitates or promotes the transport or delivery of a composition (e.g., a TDSC or nucleic acid encoding a dsDNA described herein) into a cell. For example, a carrier may be a partially or completely encapsulating agent.

As used herein, the term “chemically modified nucleotide,” as used herein with respect to DNAs, refers to a nucleotide comprising one or more structural differences relative to the canonical deoxyribonucleotides (i.e., G, T, C, and A). A chemically modified nucleotide may have (relative to a canonical nucleotide) a chemically modified nucleobase, a chemically modified sugar, a chemically modified phosphodiester linkage, or a combination thereof. No particular process of making is implied; for instance, a chemically modified nucleotide can be produced directly by chemical synthesis, or by covalently modifying a canonical nucleotide.

As used herein, the term “chemically modified cytosine nucleotide,” as used herein with respect to DNAs, refers to a chemically modified nucleotide wherein the nucleobase comprises a monocyclic 6-member ring in which carbon 4 is covalently bound to a nitrogen that is not one of the six members of the ring, wherein the nucleobase of the chemically modified cytosine nucleotide comprises one or more structural differences relative to canonical cytosine nucleobase. In some embodiments, the C-5 position of the nucleobase can have a substitution other than H. No particular process of making is implied.

As used herein, the term “closed end” refers to a portion of a DNA molecule positioned at one end of a double-stranded region, in which all nucleotides within the portion of the DNA molecule are covalently attached to adjacent nucleotides on either side. A closed end may, in some embodiments, include a loop comprising one or more nucleotides that are not hybridized to another nucleotide. In some embodiments, every nucleotide of the closed end is hybridized to another nucleotide. In some embodiments, a TDSC comprises a first closed end (e.g., upstream of a heterologous object sequence) and a second closed end (e.g., downstream of a heterologous object sequence).

As used herein, the term “open end” refers to a portion of a DNA molecule positioned at one end of a double-stranded region, in which at least one nucleotide (a “terminal nucleotide”) is covalently attached to exactly one other nucleotide. In some embodiments, the terminal nucleotide comprises a free 5′ phosphate. In some embodiments, the terminal nucleotide comprises a free 3′ OH. In some embodiments, in a TDSC comprising a first DNA strand and a second DNA strand, the open end comprises a first terminal nucleotide on the first DNA strand and a second terminal nucleotide on the second DNA strand. In some embodiments, a TDSC comprises a first open end (e.g., upstream of a heterologous object sequence) and a second open end (e.g., downstream of a heterologous object sequence). In some embodiments, the open end comprises a blunt end, a sticky end, or a Y-adaptor.

As used herein, the term “DNA” refers to any compound and/or substance that comprises at least two (e.g., at least 10, at least 20, at least 50, at least 100) covalently linked deoxyribonucleotides. In some embodiments, the DNA is a single oligonucleotide chain, while in other embodiments, the DNA comprises a plurality of oligonucleotide chains, while in yet other embodiments the DNA is a portion of an oligonucleotide chain. In some embodiments, DNA is a compound and/or substance that is or can be incorporated into an oligonucleotide chain via a phosphodiester linkage. In some embodiments, the DNA comprises solely canonical nucleotides. In some embodiments, the DNA comprises one or more chemically modified nucleotides. In some embodiments, at least 80%, at least 85%, at least 90%, at least 95%, at least 98%, at least 99%, or 100% of the sugars of the DNA are deoxyribose sugars. In some embodiments, the DNA was prepared by one or more of: isolation from a natural source, enzymatic synthesis by polymerization based on a complementary template (in vivo or in vitro), reproduction in a recombinant cell or system, and chemical synthesis.

As used herein, the term “DNA end form” refers to a structure comprising DNA that is situated at an end of a TDSC. In some embodiments, the DNA end form comprises a closed end. In other embodiments, the DNA end form comprises an open end. In some embodiments, the DNA end form comprises a hairpin, a loop, a Y-adaptor, a blunt end, or a sticky end. The DNA end form may comprise one or both of a single stranded region and a double stranded region. The DNA end form may comprise canonical nucleotides, chemically modified nucleotides, or a combination thereof. In some embodiments, the DNA end form comprises between 3-100 nucleotides. In some embodiments, the TDSC comprises a first DNA end form at a first end and a second DNA end form at a second end. In some embodiments, the first DNA end form and the second DNA end form of a TDSC are the same type. In some embodiments, the first DNA end form and the second DNA end form of a TDSC are different types.

As used herein, the term “exonuclease-resistant”, when used to describe a DNA, means that the DNA, if it comprises closed ends, is resistant to the exonuclease assay described in Example 10, and if it comprises an open end (e.g., two open ends), is resistant to the exonuclease assay described in Example 11.

As used herein, the term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, the term “heterologous functional sequence” refers to a nucleic acid sequence that is heterologous to an adjacent (e.g., directly adjacent) nucleic acid sequence and has one or more biological function. In some embodiments, the biological function comprises targeting to an organelle, e.g., nuclear targeting. In some embodiments, the heterologous functional sequence comprises a nuclear targeting sequence or a regulatory sequence.

As used herein, the terms “increasing” and “decreasing” refer to modulating resulting in, respectively, greater or lesser amounts, of function, expression, or activity of a metric relative to a reference. For example, subsequent to administration of a TDSC in a method described herein, the amount of the metric described herein (e.g., the level of gene expression, or a marker of innate immunity) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration, or relative to administration of a control TDSC, such as a TDSC comprising chemically modified nucleotides compared to an unmodified TDSC. Generally, the metric is measured subsequent to administration at a time that the administration has had the recited effect, e.g., at least one day, one week, one month, 3 months, or 6 months, after a treatment regimen has begun.

As used herein the term “linear” in reference to a TDSC or nucleic acid comprising dsDNA described herein, means a nucleic acid comprising two DNA strands or portions of strands which hybridize with each other (thereby forming a double stranded region), wherein the structure comprises two ends. An end may be a closed end or an open end. The two strands that hybridize with each other may be partially or completely complementary. In some embodiments, a linear TDSC consists of a single strand of DNA that is circular under denaturing conditions, wherein under physiological conditions a first portion of the strand hybridizes to a second portion of the strand (thereby forming a double stranded region), and the linear TDSC comprises a first closed end comprising a first loop and a second closed end comprising a second loop.

As used herein, the term “loop” refers to a nucleic acid sequence that is single stranded. A loop is connected at both ends by a double stranded region referred to as a “stem”, to form a “stem-loop”.

As used herein, the term “maintenance sequence” is a DNA sequence or motif that enables or facilitates retention of a DNA molecule in the nucleus through cell division. A maintenance sequence typically enables replication and/or transcription of DNA in the nucleus by interacting with proteins that facilitate chromatin looping. An example of a maintenance sequence is a scaffold/matrix attached region (S/MAR element).

As used herein, a “nuclear targeting sequence” is a DNA sequence that enables or facilitates DNA entry into a target cell nucleus. In some embodiments, the nuclear targeting sequence is a DNA sequence of Table 3.

As used herein, a “pharmaceutical composition” or “pharmaceutical preparation” is a composition or preparation which is indicated for animal, e.g., human or veterinary pharmaceutical use, for example, non-human animal or human prophylactic, diagnostic or therapeutic use. A pharmaceutical preparation comprises an active agent having a biological effect on a cell or tissue of a subject, e.g., having pharmacological activity or an effect in the mitigation, treatment, or prevention of disease, in combination with a pharmaceutically acceptable excipient or diluent. A pharmaceutical composition also means a finished dosage form or formulation of a prophylactic, diagnostic or therapeutic composition.

As used herein, the terms “peptide,” “polypeptide,” and “protein” are used interchangeably and refer to a compound comprising amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. In some embodiments, a polypeptide comprises a non-canonical amino acid residue. As used herein, the term “protelomerase sequence” refers to a nucleotide sequence capable of being generated by a protelomerase that joins a first protelomerase recognition sequence (PRS) to a second PRS. In some embodiments, the protelomerase sequence was produced by a process involving protelomerase, and in other embodiments the protelomerase sequence was produced by a process that does not involve protelomerase (e.g., by solid phase synthesis).

As used herein, a “sense strand” of a dsDNA is a strand which has the same sequence as an mRNA or pre-mRNA which encodes for a functional protein, and does not serve as a template for transcription. An “antisense strand” of a dsDNA is a strand that has a sequence complementary to an mRNA or pre-mRNA which encodes for a functional protein and/or can serve as a template for transcription.

As used herein, the term “double stranded DNA” or dsDNA means a DNA composition comprising two complementary chains of deoxyribonucleotides that base pair to each other. The two complementary strands may have perfect complementarity or may have one or more mismatches, e.g., forming bulges. Either of the two strands may, in some embodiments, have paired regions of self-complementarity that form intramolecular/intrastrand double stranded motifs in a folded configuration, for example, may form hairpin loops, junctions, bulges or internal loops. In some embodiments, the dsDNA comprises one or two closed ends. In some embodiments, the dsDNA molecule is circular or linear. In some embodiments (e.g., in a dsDNA molecule with closed ends) the two complementary chains of deoxyribonucleotides are covalently linked.

As used herein, the term “therapeutic double stranded construct” (“TDSC”) refers to a linear construct comprising DNA, wherein the construct is at least partially double stranded. A TDSC does not comprise a plasmid backbone sequence (e.g., does not comprise a bacterial origin of replication). A TDSC does not comprise a viral capsid or a viral envelope. In some embodiments, the TDSC comprises a closed end or an open end (e.g., a blunt end or a sticky end). In some embodiments, the TDSC is suitable for administration to a human subject.

As used herein, the term “proto-TDSC” refers to a construct which can be converted to a TDSC. In some embodiments, the proto-TDSC is a manufacturing intermediate which can be subjected to one or more steps (e.g., cleavage steps) to be converted into a TDSC. In some embodiments, the proto-TDSC falls within the definition of a TDSC, e.g., the proto-TDSC is a first TDSC, and can be subjected to one or more steps to be converted into a second TDSC.

As used herein, the term “terminal nucleotide” refers to a nucleotide that is covalently attached to exactly one other nucleotide. In some embodiments, the terminal nucleotide comprises a free 5′ phosphate. In some embodiments, the terminal nucleotide comprises a free 3′ OH.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “Y-adaptor” refers to a nucleic acid structure comprising a first nucleic acid region and a second nucleic acid region which are complementary (e.g., perfectly complementary) to each other; the first and second regions may hybridize to form a double stranded region. The first nucleic acid region is covalently linked to a third nucleic acid region, and the second nucleic acid region is covalently linked to a fourth nucleic acid region, and the third and fourth nucleic acid regions are not substantially complementary to each other; the third and fourth regions may be single stranded. The first nucleic acid region is 3′ of the third nucleic acid region and the second nucleic acid region is 5′ of the fourth nucleic acid region. As a result, the third and fourth regions may be situated on the same side of the double stranded regions. The Y-adaptor may be part of a TDSC.

### BRIEF DESCRIPTION OF THE DRAWINGS

FIGS. 1A-1B are a series of diagrams showing exemplary covalently-closed DNA end forms that can be included in a therapeutic double-stranded construct (TDSC) as described herein (e.g., at one or both ends of the TDSC). Shown in FIG. 1A are exemplary TDSCs comprising no loop ends (e.g., protelomerase sequences), inverted terminal repeats (ITRs), or hairpins at the ends, which can be made up of unmodified nucleotides (white symbols) or may comprise chemically modified nucleotides (gray symbols). Chemically modified nucleotides can include nucleotides modified, for example, in the backbone, sugar, or base, or nucleotides that are conjugated to a peptide or protein. In some instances, both of the DNA strands are unmodified. In some instances, both of the DNA strands are chemically modified. In some instances, the antisense strand is chemically modified. In some instances, the sense strand is chemically modified. The solid-line box in FIG. 1A indicates a dsDNA molecule that is covalently closed with hairpins at the ends, e.g., a linear, covalently closed dsDNA molecule with end forms comprising phosphorothioate modifications. The dashed-line box in FIG. 1A indicates a dsDNA molecule that is covalently closed with no loop ends, e.g., a linear, covalently closed dsDNA molecule with TelN end forms.

FIG. 2 is a series of diagrams showing double-stranded DNA constructs, including exemplary TDSCs comprising exemplary DNA end forms (e.g., at one or both ends) that are not covalently closed. Such exemplary TDSCs can comprise a Y end (e.g., a Y adaptor, e.g., as described herein). The DNA end forms can, in some instances, be made up of unmodified nucleotides (white symbols). In some instances, the DNA end forms comprise chemically modified nucleotides (gray symbols). Chemically modified nucleotides can include nucleotides modified, for example, in the backbone, sugar, or base, or nucleotides that are conjugated to a peptide or protein. In some instances, both of the DNA strands are unmodified. In some instances, both of the DNA strands are chemically modified. In some instances, the antisense strand is chemically modified. In some instances, the sense strand is chemically modified. Also shown in the upper right is an exemplary DNA construct lacking DNA end forms or chemical modifications (i.e., an unmodified double-stranded DNA molecule).

FIG. 3 is a series of diagrams showing the production of an exemplary TDSC comprising blunt end DNA end forms that include three phosphorothioate modifications between the terminal nucleotides of each strand. In brief, a hairpin structure comprising the phosphorothioate modified nucleotides at the terminal end of the stem is ligated to an A-tailed double-stranded DNA. The hairpin includes a pair of uracil nucleotides (arrows). Subsequent treatment with USER enzyme results in cleavage at the uracil positions, creating an overhang that is then trimmed back to the phosphorothioate-modified nucleotides using a nuclease (e.g., a single-strand-specific nuclease, e.g., Mung Bean nuclease). FIG. 3 discloses SEQ ID NOS 85 and 86, respectively, in order of appearance.

FIG. 4 is a series of diagrams showing the production of an exemplary TDSC comprising blunt end DNA end forms that include six phosphorothioate modifications between the terminal nucleotides of each strand. In brief, a hairpin structure comprising the phosphorothioate modified nucleotides at the terminal end of the stem is ligated to an A-tailed double-stranded DNA. The hairpin includes a pair of uracil nucleotides (arrows). Subsequent treatment with USER enzyme results in cleavage at the uracil positions, creating an overhang that is then trimmed back to the phosphorothioate-modified nucleotides using a nuclease (e.g., a single-strand-specific nuclease, e.g., Mung Bean nuclease). FIG. 4 discloses SEQ ID NOS 87 and 88, respectively, in order of appearance.

FIG. 5 is a series of diagrams showing the production of an exemplary TDSC comprising a Y adaptor DNA end form at each end. In brief, a hairpin structure comprising a uracil nucleotide in the loop region is ligated to an A-tailed double-stranded DNA. In an embodiment, the loop region comprises phosphorothioate modifications between the nucleotides. Subsequent treatment with USER enzyme results in cleavage of the uracil in the loop, resulting in two non-hybridized strands that form the Y adaptor structure at the end of the dsDNA. FIG. 5 discloses SEQ ID NOS 89-92, 92 and 91, respectively, in order of appearance.

FIG. 6 is a series of diagrams showing exemplary covalently-closed DNA end forms that can be included in a TDSC as described herein. The DNA forms include, for example, a small loop adaptor comprising a hairpin, a large loop adaptor (e.g., comprising a hairpin comprising, in its single-stranded loop region, one or more functional elements, such as a CT3 ssDNA sequence), and a no loop adaptor, in which every nucleotide of the DNA end form is hybridized to another nucleotide and in which the end is covalently closed. Each of these exemplary end forms can, for example, be ligated to a double-stranded DNA to form one end of a TDSC as described herein. FIG. 6 discloses SEQ ID NOS 93-95, respectively, in order of appearance.

FIG. 7A depicts an agarose gel showing TDSCs after exonuclease treatment. FIG. 7B shows the TDSC designated 6a, corresponding to lanes 3 and 4 of the agarose gel. FIG. 7C shows the TDSC designated 3a, corresponding to lanes 5 and 6 of the agarose gel. FIG. 7D shows one end of the TDSC designated Ya, corresponding to lanes 7 and 8 of the agarose gel. FIG. 7D discloses SEQ ID NOS 96 and 97, respectively, in order of appearance.

FIG. 8 is a diagram depicting production of covalently closed TDSCs with end forms comprising six phosphorothioate modifications (P6 forms). FIG. 8 discloses SEQ ID NOS 98 and 99, respectively, in order of appearance.

FIG. 9 is a diagram depicting production of covalently closed TDSCs with TelN end forms. FIG. 9 discloses SEQ ID NOS 100, 100 and 101, respectively, in order of appearance.

FIG. 10 is a diagram depicting an exemplary method of production of circular dsDNA molecules. A linear dsDNA molecule may be contacted with a restriction enzyme (e.g., KpnI) that creates compatible sticky ends which may then be joined to each other by ligation, producing a circular dsDNA. FIG. 10 discloses SEQ ID NOS 102-105, respectively, in order of appearance.

FIG. 11 shows a fragment analyzer trace of circular dsDNA produced in a reaction using 25% 5-formyl-dCTP.

FIG. 12 shows a fragment analyzer trace of covalently closed TDSCs with end forms comprising phosphorothioate modifications produced in a reaction using 25% 5-formyl-dCTP.

FIG. 13 shows a fragment analyzer trace of covalently closed TDSCs with TelN end forms produced in a reaction using 25% 5-formyl-dCTP.

FIGS. 14A and 14B are graphs showing expression of the reporter protein mCherry in HEKa cells lipofected with covalently closed TDSCs comprising TelN end forms (TelN form), covalently closed TDSCs comprising end forms with six phosphorothioate modifications in each end form (P6 form), or circular dsDNA molecules (cdsDNA form). The TDSCs and circular dsDNA molecules were produced in a reaction using unmodified cytosines or 25% 5-formyl-dCTP. FIG. 14A shows the percentage of mCherry-expressing cells, and FIG. 14B shows the total fluorescence, defined as percentage of expressing cells multiplied by mean fluorescence intensity.

FIGS. 15A-15C is a series of graphs showing the mRNA levels of IFNβ (FIG. 15A), CXCL10 (FIG. 15B), and IL6 (FIG. 15C) in HEKa cells following lipofection with covalently closed TDSCs comprising TelN end forms (TelN form), covalently closed TDSCs comprising end forms with six phosphorothioate modifications in each end form (P6 form), or circular dsDNA molecules (cdsDNA form). The TDSCs and circular dsDNA molecules were produced in a reaction using unmodified cytosines or 25% 5-formyl-dCTP. RNA expression was normalized to GAPDH and expressed as fold-changes relative to the method control (DNA-free transfection).

FIGS. 16A-16C is a series of graphs showing the mRNA levels of IFNβ (FIG. 16A), CXCL10 (FIG. 16B), and IL6 (FIG. 16C) in THP1 cells following lipofection with covalently closed TDSCs comprising TelN end forms (TelN form), covalently closed TDSCs comprising end forms with six phosphorothioate modifications in each end form (P6 form), or circular dsDNA molecules (cdsDNA form). The TDSCs and circular dsDNA molecules were produced in a reaction using unmodified cytosines or 25% 5-formyl-dCTP. RNA expression was normalized to GAPDH and expressed as fold-changes relative to the method control (DNA-free transfection).

FIG. 17 is a scatterplot showing the innate immune response of HEKa cells to TDSCs comprising phosphorothioated end adapters and the indicated modification at the C-5 position of cytosine (i.e., 5-formylcytosine, 5-carboxycytosine, 5-methylcytosine, or 5-hydroxymethylcytosine). X-axis represents reduction in interferon signaling, defined as the average fold-change reduction of markers IFNB and CXCL10 relative to a TDSC produced using unmodified cytosines. Y-axis represents reduction in inflammatory cytokine signaling, defined as the average fold-change reduction of markers IL6 and TNFa relative to a TDSC produced using unmodified cytosines.

### DETAILED DESCRIPTION

This disclosure relates to compositions and methods for providing an effector, e.g., a therapeutic effector, to a cell, tissue or subject, e.g., in vivo or in vitro. The effector may be a DNA sequence, a polypeptide, e.g., a therapeutic protein, or an RNA, e.g., a regulatory RNA or an mRNA.

### Elements of DNA Constructs

The TDSCs or nucleic acids comprising dsDNA described herein contain elements sufficient to deliver an effector sequence to a target cell, tissue or subject. In some embodiments, the effector sequence is a DNA sequence. In some embodiments, the TDSC drives expression of an effector, e.g., comprises a promoter and a sequence encoding an RNA or a polypeptide, e.g., a therapeutic RNA or polypeptide. In some embodiments, the DNA constructs described herein further contain one or both of: a nuclear targeting sequence and a maintenance sequence. While many of the embodiments herein refer to a TDSC, it is understood that as applicable an embodiment that refers to a TDSC may also apply to a nucleic acid comprising dsDNA.

### Exonuclease-Resistant DNA End Forms

The TDSCs or nucleic acids comprising dsDNA described herein comprise a DNA end form at each end of the double-stranded DNA molecule. The DNA end forms described herein can, in some instances, comprise a closed end, wherein every nucleotide of the DNA end form is covalently attached to two other nucleotides of the DNA end form. In other instances, the DNA end forms described herein comprise an open end comprising at least one nucleotide that are only covalently attached to one other nucleotide of the DNA end form. The DNA end forms are generally exonuclease resistant. In some instances, a DNA end form comprising a closed end (e.g., a covalently closed end) is resistant to the exonuclease assay described in Example 10. In some instances, a DNA end form comprising an open end (e.g., such as a Y adaptor, blunt end, or sticky end, e.g., as described herein) is resistant to the exonuclease assay described in Example 11.

### Closed Ends, e.g., Hairpins

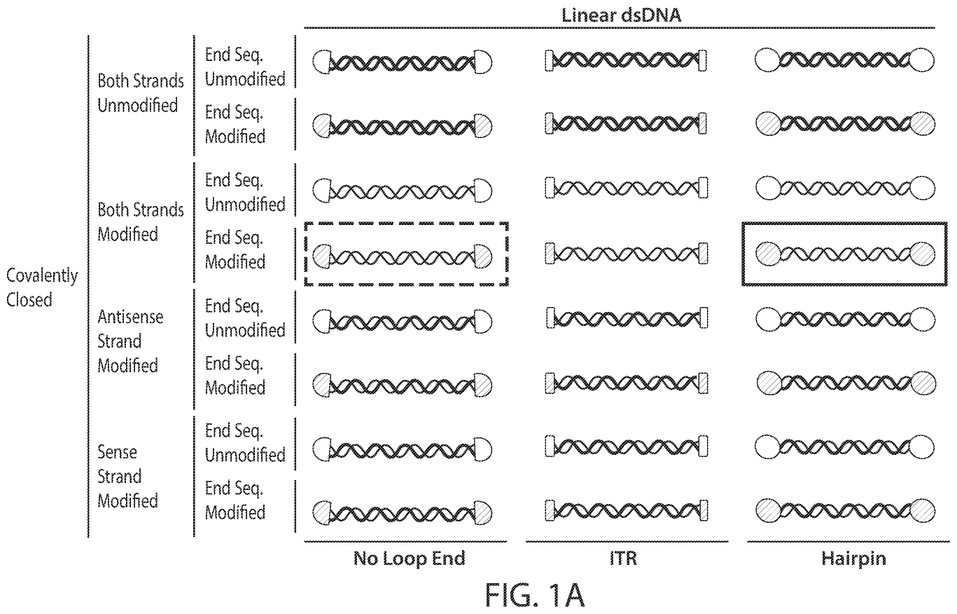

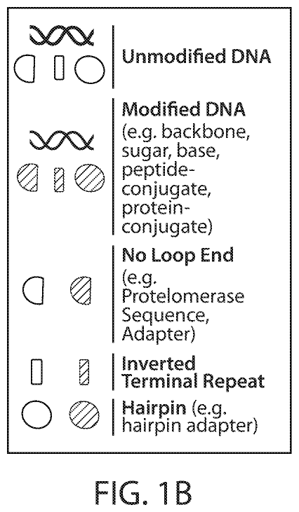

In some embodiments, an exonuclease-resistant DNA end form comprises a DNA hairpin. A hairpin generally comprises a single-stranded loop region covalently attached at both the 5′ and 3′ ends to a double-stranded stalk region. In certain embodiments, the single-stranded loop region comprises one or more nucleotides (e.g., 1-2, 2-5, 5-10, 10-15, 15-20, 20-25, 25-30, 30-35, or 35-40 nucleotides) that are not hybridized to another nucleotide. Exemplary hairpin structures, and exemplary TDSCs comprising hairpins, are shown in FIG. 1A.

In certain embodiments, the single-stranded loop region comprises one or more functional elements (e.g., a nuclear import sequence (e.g., a CT3 ssDNA sequence), or a regulatory sequence. In embodiments, a functional element comprised in the single-stranded loop region is heterologous to one or more other elements of the DNA end form and/or a TDSC comprising the DNA end form. In certain embodiments, the single-stranded loop region of a hairpin loop is less than about 5, 10, 15, 20, 25, 26, 27, 28, 29, or 30 nucleotides in length.

In embodiments, the hairpin is comprised in a TDSC having a doggybone conformation. In embodiments, the hairpin comprises a protelomerase sequence (e.g., as described herein). In embodiments, the protelomerase sequence is produced by TelN protelomerase, ResT protelomerase, Tel PY54 protelomerase, or TelK protelomerase digestion. In embodiments, the protelomerase sequence is less than about 15, 20, 25, 26, 27, 28, 29, or 30 nucleotides in length. In embodiments, the protelomerase sequences are between about 28 (e.g., 25, 26, 27, 28, 29, 30, 31, 32, 33, 34, or 35) nucleotides and about 56 (e.g., 50, 51, 52, 53, 54, 55, 56, 57, 58, 59, or 60) nucleotides in length. In embodiments, the protelomerase sequences are greater than about 56 (e.g., greater than 50, 51, 52, 53, 54, 55, 56, 57, 58, 59, 60, 65, 70, 75, 80, 90, or 100) nucleotides in length.

A hairpin can be attached to one or both ends of a double-stranded DNA molecule (e.g., a proto-TDSC as described herein), for example, by ligation (e.g., as described herein). In some embodiments, a TDSC as described herein comprises, at one or both ends, a DNA hairpin loop. In some embodiments, the upstream exonuclease-resistant DNA end form of a TDSC as described herein comprises a DNA hairpin loop. In some embodiments, the downstream exonuclease-resistant DNA end form of a TDSC as described herein comprises a DNA hairpin loop.

In certain embodiments, a DNA hairpin loop comprises one or more unmodified nucleotides. In embodiments, a DNA hairpin loop consists entirely of unmodified nucleotides. In certain embodiments, a DNA hairpin loop comprises one or more chemically modified nucleotides (e.g., phosphorothioate-modified nucleotides, e.g., as described herein). In embodiments, a DNA hairpin loop consists entirely of chemically modified nucleotides (e.g., phosphorothioate-modified nucleotides, e.g., as described herein).

In certain embodiments, the single-stranded loop region of a DNA hairpin loop comprises one or more chemically modified nucleotides (e.g., phosphorothioate-modified nucleotides, e.g., as described herein). In embodiments, at least 50%, 60%, 70%, 75%, 80%, 85%, 90%, 95%, 96%, 97%, or 99% of the nucleotides in the single-stranded loop region are chemically modified nucleotides (e.g., phosphorothioate-modified nucleotides, e.g., as described herein). In embodiments, the single-stranded loop region of a DNA hairpin loop consists entirely of chemically modified nucleotides (e.g., phosphorothioate-modified nucleotides, e.g., as described herein). In certain embodiments, the single-stranded loop region of a DNA hairpin loop comprises one or more unmodified nucleotides. In embodiments, the single-stranded loop region of a DNA hairpin loop consists entirely of unmodified nucleotides.

In certain embodiments, the double-stranded stalk region of a DNA hairpin loop comprises one or more unmodified nucleotides. In embodiments, the double-stranded stalk region of a DNA hairpin loop consists entirely of unmodified nucleotides. In certain embodiments, the double-stranded stalk region of a DNA hairpin loop comprises one or more chemically modified nucleotides (e.g., phosphorothioate-modified nucleotides, e.g., as described herein). In embodiments, at least 50%, 60%, 70%, 75%, 80%, 85%, 90%, 95%, 96%, 97%, or 99% of the nucleotides in the double-stranded stalk region are modified nucleotides (e.g., phosphorothioate-modified nucleotides, e.g., as described herein). In embodiments, the double-stranded stalk region of a DNA hairpin loop consists entirely of chemically modified nucleotides (e.g., phosphorothioate-modified nucleotides, e.g., as described herein).

In embodiments, the single-stranded loop region of a DNA hairpin loop comprises one or more chemically modified nucleotides (e.g., phosphorothioate-modified nucleotides, e.g., as described herein) and the double-stranded stalk region comprises one or more unmodified nucleotides. In embodiments, at least 50%, 60%, 70%, 75%, 80%, 85%, 90%, 95%, 96%, 97%, or 99% of the nucleotides in the single-stranded loop region are chemically modified nucleotides (e.g., phosphorothioate-modified nucleotides, e.g., as described herein). In embodiments, the single-stranded loop region of a DNA hairpin loop consists entirely of chemically modified nucleotides (e.g., phosphorothioate-modified nucleotides, e.g., as described herein) and the double-stranded stalk region consists entirely of unmodified nucleotides.

### Y-Adaptors

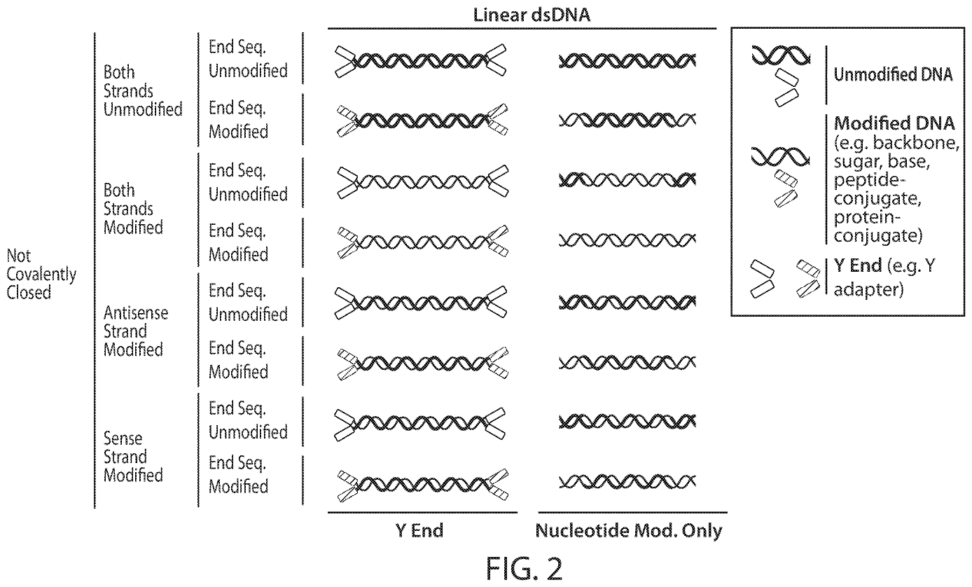

In some embodiments, an exonuclease-resistant DNA end form as described herein comprises a Y-adaptor. As described herein, a Y-adaptor generally comprises a pair of single-stranded DNA regions, each attached at one end to a strand of a double-stranded DNA region, thereby forming a “Y” shape (wherein the base of the “Y” represents the double-stranded DNA region, and each of the upper prongs of the “Y” represents the two single-stranded DNA region). Exemplary Y-adaptor structures and exemplary TDSCs comprising Y-adaptors are shown in FIG. 2.

In some embodiments, a Y-adaptor is produced by attaching a hairpin loop comprising a single-stranded region comprising a cleavable moiety (e.g., a uracil nucleotide) to the end of a double-stranded DNA region (e.g., via ligation). The cleavable moiety can then be cleaved (e.g., by treatment with an enzyme capable of cleaving the cleavable moiety, e.g., a USER enzyme) to produce the two single-stranded DNA regions of the Y-adaptor.

In certain embodiments, a single-stranded DNA region (e.g., one or both single-stranded DNA regions) of a Y-adaptor comprises one or more chemically modified nucleotides (e.g., phosphorothioate-modified nucleotides, e.g., as described herein). In embodiments, at least 50%, 60%, 70%, 75%, 80%, 85%, 90%, 95%, 96%, 97%, or 99% of the nucleotides in the single-stranded DNA region are chemically modified nucleotides (e.g., phosphorothioate-modified nucleotides, e.g., as described herein). In embodiments, a single-stranded DNA region (e.g., one or both single-stranded DNA regions) of a Y-adaptor consists entirely of chemically modified nucleotides (e.g., phosphorothioate-modified nucleotides, e.g., as described herein). In certain embodiments, a single-stranded DNA region (e.g., one or both single-stranded DNA regions) of a Y-adaptor comprises one or more unmodified nucleotides.

In embodiments, a single-stranded DNA region (e.g., one or both single-stranded DNA regions) of a Y-adaptor comprises one or more chemically modified nucleotides (e.g., phosphorothioate-modified nucleotides, e.g., as described herein) and a double-stranded DNA region of the Y-adaptor comprises one or more unmodified nucleotides. In embodiments, at least 50%, 60%, 70%, 75%, 80%, 85%, 90%, 95%, 96%, 97%, or 99% of the nucleotides in the single-stranded DNA region or regions are chemically modified nucleotides (e.g., phosphorothioate-modified nucleotides, e.g., as described herein). In embodiments, a single-stranded DNA region (e.g., one or both single-stranded DNA regions) of a Y-adaptor consists entirely of chemically modified nucleotides (e.g., phosphorothioate-modified nucleotides, e.g., as described herein) and the double-stranded DNA region of the Y-adaptor consists entirely of unmodified nucleotides.

### No Loop Closed DNA End Forms

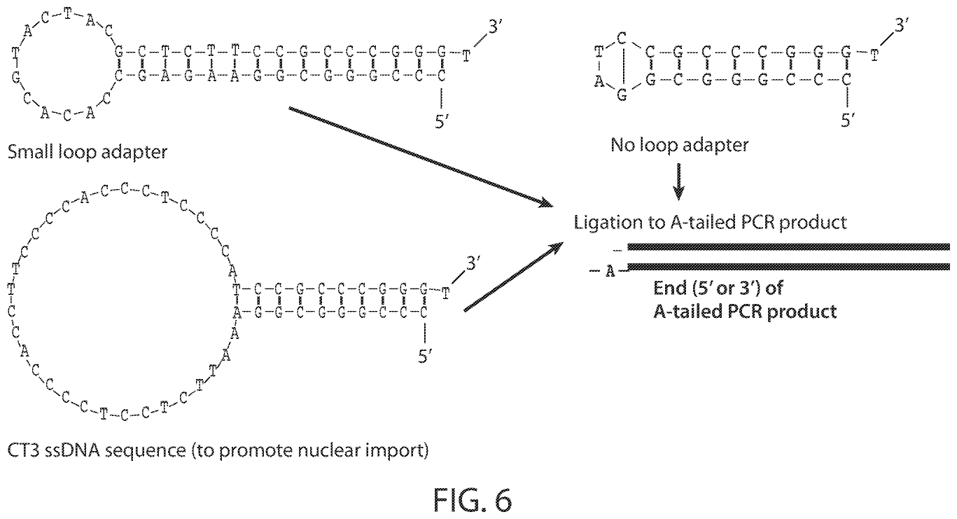

In some embodiments, a TDSC as described herein comprises an exonuclease-resistant DNA end form that is covalently closed but does not include a hairpin loop. For example, in certain embodiments, every nucleotide of a covalently-closed DNA end form is hybridized to another nucleotide (e.g., as shown in the exemplary “No loop adapter” of FIG. 6). In certain embodiments, the covalently-closed DNA end form comprises a first region and a second region, wherein the first region is capable of hybridizing in its entirety to the second region (e.g., wherein the first region is complementary to the second region) and wherein the 3′ end of the first region is covalently attached to the 5′ end of the second region. In embodiments, a covalently-closed DNA end form as described herein can be attached to one end of a proto-TDSC as described herein, e.g., by ligation.

### Open DNA End Forms

In some embodiments, a TDSC as described herein comprises an exonuclease-resistant DNA end form that is not covalently closed. In certain embodiments, the DNA end form comprises a blunt end (e.g., a blunt end comprising one or more chemical modifications as described herein) or a sticky end (e.g., a sticky end comprising one or more chemical modifications as described herein).

In certain embodiments, the open DNA end form is produced by nuclease digestion of a covalently closed DNA end form, such as a DNA hairpin. In embodiments, the DNA hairpin comprises a double-stranded stalk region comprising a cleavable moiety (e.g., a uracil nucleotide) on each strand, and the DNA hairpin is then contacted with an enzyme capable of cleaving the cleavable moieties (e.g., a USER enzyme). In embodiments, this results in the formation of a sticky end comprising an overhang. In embodiments, the overhang is digested with an enzyme (e.g., a single-stranded specific nuclease, e.g., a Mung Bean nuclease) to form a blunt end.

In certain embodiments, a DNA end form comprising a blunt end comprises one or more chemically modified nucleotides (e.g., phosphorothioate-modified nucleotides, e.g., as described herein). In embodiments, at least 50%, 60%, 70%, 75%, 80%, 85%, 90%, 95%, 96%, 97%, or 99% of the nucleotides in the DNA end form comprising a blunt end are chemically modified nucleotides (e.g., phosphorothioate-modified nucleotides, e.g., as described herein). In embodiments, the DNA end form comprising a blunt end consists entirely of chemically modified nucleotides (e.g., phosphorothioate-modified nucleotides, e.g., as described herein). In embodiments, the terminal base pair of the DNA end form comprising a blunt end comprises a chemically modified nucleotide (e.g., one or both nucleotides of the base pair are chemically modified), e.g., a phosphorothioate-modified nucleotide, e.g., as described herein. In embodiments, a plurality of base pairs (e.g., 2, 3, 4, 5, or 6 base pairs) at the terminal end of the DNA end form comprise chemically modified nucleotides (e.g., one or both nucleotides of the base pair are chemically modified), e.g., phosphorothioate-modified nucleotides, e.g., as described herein. In an embodiment, the three base pairs at the terminal end of the DNA end form comprise chemically modified nucleotides (e.g., one or both nucleotides of the base pair are chemically modified), e.g., phosphorothioate-modified nucleotides, e.g., as described herein. In an embodiment, the six base pairs at the terminal end of the DNA end form comprise chemically modified nucleotides (e.g., one or both nucleotides of the base pair are chemically modified), e.g., phosphorothioate-modified nucleotides, e.g., as described herein.

In certain embodiments, a DNA end form comprising a sticky end comprises one or more chemically modified nucleotides (e.g., phosphorothioate-modified nucleotides, e.g., as described herein). In embodiments, at least 50%, 60%, 70%, 75%, 80%, 85%, 90%, 95%, 96%, 97%, or 99% of the nucleotides in the DNA end form comprising a sticky end are chemically modified nucleotides (e.g., phosphorothioate-modified nucleotides, e.g., as described herein). In embodiments, the DNA end form comprising a sticky end consists entirely of chemically modified nucleotides (e.g., phosphorothioate-modified nucleotides, e.g., as described herein). In embodiments, a terminal nucleotide of the DNA end form comprising a sticky end comprises a chemically modified nucleotide (e.g., one or both nucleotides of the base pair are chemically modified), e.g., a phosphorothioate-modified nucleotide, e.g., as described herein. In embodiments, the overhang region of the sticky end of a DNA end form comprises one or more chemically modified nucleotide, e.g., phosphorothioate-modified nucleotides, e.g., as described herein.

### Inverted Terminal Repeats (ITRs)

In some embodiments, a TDSC as described herein comprises an exonuclease-resistant DNA end form comprising an inverted terminal repeat (ITR). In some embodiments, the ITR is an ITR from a virus, e.g., an adenovirus or an adeno-associated virus (AAV). In some embodiments, the ITR comprises a nucleic acid sequence having at least 75%, 80%, 85%, 90%, 95%, 96%, 97%, 98%, 99%, or 100% sequence identity to an ITR sequence from a virus, e.g., an adenovirus or an adeno-associated virus (AAV). In certain embodiments, the ITR comprises an origin of replication (e.g., a viral origin of replication). In embodiments, a TDSC as described herein comprises an exonuclease-resistant DNA end form comprising an ITR (e.g., as described herein) at each end. In some embodiments, a TDSC does not comprise an ITR.

### Promoters and Other Regulatory Sequences

The TDSC or nucleic acid comprising dsDNA described herein may contain a promoter (a DNA sequence at which RNA polymerase and transcription factors bind to, directly or indirectly, to initiate transcription) operably linked to an effector sequence. A promoter may be found in nature operably linked to the effector sequence, or may be heterologous to the effector sequence. A promoter described herein may be native to the target cell or tissue, or heterologous to the target cell or tissue. A promoter may be constitutive, inducible and/or tissue-specific.

Examples of constitutive promoters include the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), the SV40 promoter, the dihydrofolate reductase promoter, the beta-actin promoter, the phosphoglycerol kinase (PGK) promoter, and the EF1alpha promoter.

Inducible promoters allow regulation of expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of sources. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the native promoter for the sequence encoding the effector can be used.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a alpha-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptoralpha.-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be known to the skilled artisan.

Examples of tissue/cell specific promoters are listed in Table 1:

TABLE 1
Tissue or cell specific promoters

| | | Accession Number; Human |
|---|---|---|
| Tissue/Cell | Promoter | Genome Coordinate (hg38) |
| Skeletal muscle | ACTA1 | NM_001100; chr1: 229,439,090- |
| | | 229,432,090 |
| Melanoma | TYR | NM_000372; chr11: 89,300,750- |
| | | 89,293,750 |
| Hepatoma | a-fetoprotein | NM_001354717; chr4: 73,461,175- |
| | | 73,454,175 |
| Mammary carcinoma | Mucin 1 | NM_001371720; |
| | | chr1: 155,197,900-155,190,900 |
| Prostate Cancer | KLK3 | NM_001648; chr19: 50,865,760- |
| | | 50,858,760 |
| Neuronal cells | ENO2 | NM_001975; chr12: 6,928,700- |
| | | 6,921,700 |
| Response to Hypoxia | HIF-1 alpha | NM_001530; chr14: 61,753,200- |
| | | 61,746,200 |
| Retinoblastoma | E2F1 | NM_005225; chr20: 33,691,380- |
| | | 33,684,380 |
| Ionizing radiation | EGR-1 | NM_001964; chr5: 138,474,303- |
| | | 138,467,303 |
| Oncogene | ErbB2 | NM_004448; chr17: 39,735,530- |
| | | 39,728,530 |
| Endothelial cells | vWF | NM_000552; chr12: 6,129,670- |
| | | 6,122,670 |
| Endothelial cells | FLT-1 | NM_002019; chr13: 28,500,100- |
| | | 28,493,100 |
| Endothelial cells | ICAM-2 | NM_001099786; |
| | | chr17: 64,025,630-64,018,630 |
| Retinal pigment | VMD2 | NM_004183; chr11: 61,972,630- |
| epithelium | | 61,965,630 |
| Rod cells | RHO | NM_000539; chr3: 129,540,350- |
| | | 129,533,350 |
| Cone cells | Red/green opsin | NM_020061; chrX: 154,164,030- |
| | (OPN1LW) | 154,157,030 |
| Ganglion cells | Thymocyte antigen | NM_006288; chr11: 119,428,150- |
| | (Thy1) | 119,421,150 |
| T cells | TIM3 | NM_032782; chr5: 157,114,050- |
| | | 157,107,050 |
| T cells | FOXP3 | NM_014009; chrX: 49,269,700- |
| | | 49,262,700 |
| PBMCs | Vβ6.7 | ENST00000390373.2; |
| | | chr7: 142,493,295-142,486,295 |
| Cell cycle | Cdk1 | NM_001786; chr10: 60,799,850- |
| | | 60,792,850 |

The constructs described herein may also include other native or heterologous expression control elements, such as enhancer elements, polyadenylation sites or Kozak consensus sequences.

### Effector Sequence

The effector sequence of a TDSC or nucleic acid comprising dsDNA described herein may be, e.g., a functional DNA sequence, e.g., a therapeutically functional DNA sequence; a DNA sequence encoding a therapeutic peptide, polypeptide or protein; or a DNA sequence encoding a therapeutic RNA (e.g., a non-coding RNA).

### DNA Effectors:

A therapeutically functional DNA sequence may be a DNA sequence that forms a functional structure, e.g., a DNA sequence comprising a DNA aptamer, DNAzyme or allele-specific oligonucleotide (a DNA ASO). A therapeutically functional DNA sequence may not have a promoter operably linked. In embodiments, a TDSC or nucleic acid comprising dsDNA described herein may include one or a plurality of functional DNA sequences, e.g., 2, 3, 4, 5, 6, or more sequences, which may be the same or different.

### Polypeptide Effectors:

A DNA sequence encoding a therapeutic polypeptide may be a DNA sequence encoding one or more effector which is a peptide, protein, or combinations thereof. For example, the DNA sequence encodes an mRNA. The peptide or protein may be: a DNA binding protein; an RNA binding protein; a transporter; a transcription factor; a translation factor; a ribosomal protein; a chromatin remodeling factor; an epigenetic modifying factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g. a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; a protease; an integrase; a recombinase; a topoisomerase; a gyrase; a helicase; a lysosomal acid hydrolase); an antibody (e.g., an intact antibody, a fragment thereof, or a nanobody); a signaling peptide; a receptor ligand; a receptor; a clotting factor; a coagulation factor; a structural protein; a caspase; a membrane protein; a mitochondrial protein; a nuclear protein; an engineered binder such as a centyrin, darpin, or adnectin. See, e.g., Gebauer & Skerra. 2020. Annual Review of Pharmacology and Toxicology 60:1, 391-415.

In embodiments, a TDSC or nucleic acid comprising dsDNA described herein may include one or a plurality of sequences encoding a polypeptide, e.g., 2, 3, 4, 5, 6, or more sequences encoding a polypeptide. Each of the plurality may encode the same or different protein. For example, a TDSC or sequence described herein may include multiple sequences encoding multiple proteins, e.g., a plurality of proteins in a biological pathway.

In some embodiments, a TDSC or sequence described herein may include a plurality of sequences encoding a polypeptide, e.g., 2, 3, 4, 5, 6, or more sequences encoding a polypeptide, separated by a self-cleaving peptide, e.g., P2A, T2A, E2A or F2A. self-cleaving peptides are 18-22 amino acids long, and can induce ribosomal skipping during protein translation so that two polypeptides can be encoded in the same transcript. Each of the polypeptides may encode the same or different protein. In one embodiment, a TDSC or sequence described herein may include a promoter followed by a sequence encoding a first polypeptide of interest, a sequence encoding a 2A self-cleaving peptide, a sequence encoding a second polypeptide of interest, and a polyA site. In another embodiment, a TDSC or sequence described herein may include a promoter followed by a sequence encoding the first polypeptide of interest, a first 2A self-cleaving peptide, a second polypeptide of interest, a sequence encoding a second 2A self-cleaving peptide, a sequence encoding a third polypeptide of interest, and a polyA site.

In some embodiments, the effector comprises a cell penetrating polypeptide. In some embodiments, the effector is a fusion protein that comprises a cell penetrating polypeptide and a second amino acid sequence.

### RNA Effectors:

An effector sequence may be a DNA sequence encoding a non-coding RNA, e.g., one or more of a short interfering RNA (siRNA), a microRNA (miRNA), long non-coding RNA, a piwi-interacting RNA (piRNA), a small nucleolar RNA (snoRNA), a small Cajal body-specific RNA (scaRNA), a transfer RNA (tRNA), a ribosomal RNA (rRNA), an RNA aptamer, and a small nuclear RNA (snRNA).

In some embodiments, the TDSC or nucleic acid comprising dsDNA disclosed herein comprises one or more expression sequences that encode a regulatory RNA, e.g., an RNA that modifies expression of an endogenous gene and/or an exogenous gene. In some embodiments, the TDSC or sequence disclosed herein can comprise a sequence that is antisense to a regulatory nucleic acid like a non-coding RNA, such as, but not limited to, tRNA, lncRNA, miRNA, IRNA, snRNA, microRNA, siRNA, piRNA, snoRNA, snRNA, exRNA, scaRNA, Y RNA, and hnRNA. In one embodiment, the regulatory nucleic acid targets a host gene. A regulatory nucleic acid may include, but is not limited to, a nucleic acid that hybridizes to an endogenous gene, e.g., an antisense RNA, a guide RNA, a nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In one embodiment, the sequence is an miRNA. In some embodiments, the regulatory nucleic acid targets a sense strand of a host gene. In some embodiments, the regulatory nucleic acid targets an antisense strand of a host gene.

In some embodiments, the TDSC or sequence disclosed herein encodes a guide RNA. Guide RNA sequences are generally designed to have a sequence having a length of between 15-30 nucleotides (e.g., 17, 19, 20, 21, 24 nucleotides) that is complementary to the targeted nucleic acid sequence, and a region that facilitates complex formation (e.g., with a tracrRNA or a nuclease). Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991. The gRNA may recognize specific DNA sequences (e.g., sequences adjacent to or within a promoter, enhancer, silencer, or repressor of a gene). In one embodiment, the gRNA is used as part of a CRISPR system for gene editing. For the purposes of gene editing, the TDSC or sequence disclosed herein may be designed to include one or multiple sequences encoding guide RNA sequences corresponding to a desired target DNA sequence; see, for example, Cong et al. (2013) Science, 339:819-823; Ran et al. (2013) Nature Protocols, 8:2281-2308.

A TDSC or sequence disclosed may encode certain regulatory nucleic acids that can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. Such RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207), RNA antisense oligonucleotides (RNA ASOs).

In one embodiment, the TDSC or sequence disclosed herein comprises a sequence comprising a sense strand of a lncRNA. In one embodiment, the TDSC or sequence disclosed herein comprises a sequence encoding an antisense strand of a lncRNA.

The TDSC or sequence disclosed herein may encode a regulatory nucleic acid substantially complementary, or fully complementary, to a fragment of an endogenous gene or gene product (e.g., mRNA). The regulatory nucleic acids may complement sequences at the boundary between introns and exons, in between exons, or adjacent to exon, to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid comprises a protein-binding site that can bind to a protein that participates in regulation of expression of an endogenous gene or an exogenous gene.

The length of a TDSC or sequence disclosed herein that may encode a regulatory nucleic acid that hybridizes to a transcript of interest and may be, for instance, between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

A TDSC or sequence disclosed herein may encode a micro-RNA (miRNA) molecule identical to about 5 to about 30 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search. In some embodiments, the TDSC or sequence disclosed herein encodes at least one miRNA, e.g., 2, 3, 4, 5, 6, or more. In some embodiments, the TDSC or sequence disclosed herein comprises a sequence that encodes an miRNA having at least about 75%, 80%, 85%, 90% 95%, 96%, 97%, 98%, 99% or 100% nucleotide sequence identity to any one of the nucleotide sequences or a sequence that is complementary to a target sequence. Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed by technologies known in the art. In addition, there are computational tools that increase the chance of finding effective and specific sequence motifs (see, e.g., Lagana et al., Methods Mol. Bio., 2015, 1269:393-412).

The TDSC or sequence disclosed herein may modulate expression of RNA encoded by a gene. Because multiple genes can share some degree of sequence homology with each other, in some embodiments, the TDSC or sequence disclosed herein can be designed to target a class of genes with sufficient sequence homology. In some embodiments, the TDSC or sequence disclosed herein can contain a sequence that has complementarity to sequences that are shared amongst different gene targets or are unique for a specific gene target. In some embodiments, the TDSC or sequence disclosed herein can be designed to target conserved regions of an RNA sequence having homology between several genes thereby targeting several genes in a gene family (e.g., different gene isoforms, splice variants, mutant genes, etc.). In some embodiments, the TDSC or sequence disclosed herein can be designed to target a sequence that is unique to a specific RNA sequence of a single gene.

In embodiments, the effector sequence encoding a regulatory RNA has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the effector sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

In some embodiments, a TDSC or sequence disclosed herein comprises one or more of the features described hereinabove, e.g., one or more structural DNA sequence, a sequence encoding one or more peptides or proteins, a sequence encoding one or more regulatory element, a sequence encoding one or more regulatory nucleic acids, e.g., one or more non-coding RNAs, other expression sequences, and any combination of the aforementioned. A construct described herein may have one or a plurality of effector sequences, e.g., 2, 3, 4, 5 or more effector sequences. In the case of a plurality of effector sequences in a single construct, the effector sequences may be the same or different.

In one embodiment, the TDSC includes a therapeutically functional, structural DNA sequence. In one embodiment, the TDSC includes a promoter and a sequence encoding a therapeutic peptide, polypeptide, or protein described herein. In one embodiment, the TDSC includes a promoter and a sequence encoding a regulatory RNA described herein.

In some embodiments, the effector sequence that encodes a polypeptide or protein is codon optimized, e.g., codon optimized for expression in a mammal, e.g., a human. In general, codon optimization means modifying a nucleic acid sequence for enhanced expression in the host cells of interest by replacing at least one codon (e.g., one or more, e.g., 1, 2, 3, 4, 5, 10, 15, 20, 25, 50, or more codons; e.g., at least 1%, 5%, 10%, 20%, 25%, 50%, 60%, 70%, 80%, 90% or 100%) of the native sequence with codons that are more frequently or most frequently used in the genes of that host cell while maintaining the native amino acid sequence. Codon usage tables are available, for example, at the “Codon Usage Database” available at http://www.kazusa.or.jp/codon/. These tables can be adapted in a number of ways, see, e.g., Nakamura et al., 2000, Nucl. Acids Res. 28:292. Computer algorithms for codon optimizing a particular sequence for expression in a particular host cell are also available, such as Gene Forge.

### Nuclear Targeting Sequences (NTS)

A TDSC or nucleic acid comprising dsDNA (e.g., as disclosed herein) may include a nuclear targeting sequence (NTS) that facilitates transport of DNA from the cytoplasm into the nucleus of a cell. An NTS includes binding sites to proteins (e.g., transcription factors, chaperones, etc.) which bind to importin which transports cargo into the nucleus via the nuclear pore complex. In embodiments, an NTS may function generally (e.g. SV40 enhancer NTS). In other embodiments, NTS's may be cell or tissue specific, e.g., containing binding sites for transcription factors expressed in unique cell types that may target a TDSC described herein to the nucleus in a cell-specific manner (e.g., SRF, Nkx3). An NTS can be functional in multiple locations in a TDSC described herein, e.g., before the promoter and/or after the effector sequence.

An NTS may be viral or non-viral derived. NTSs are described, e.g., in Le Guen et al. 2021. Nucleic Acids Vol. 24:477-486. Examples of NTS's are disclosed in Table 2:

TABLE 2
Exemplary nuclear targeting sequences

| Viral/Non-viral | Name | Sequence |
|---|---|---|
| Viral | SV40 | 5′-cccaagaagaagaggaaag |
| | | tc-3′ (SEQ ID NO: 1) |
| Non-viral | 3NF | 5′-ctggggactttccagcctg |
| | | gggactttccagctgggacttt |
| | | ccagg-3′ |
| | | (SEQ ID NO: 106) |

In some embodiments, the NTS has a sequence according to Table 2, or a functional sequence having at least 80%, 90%, 95%, 96%, 97%, 98%, or 99% identity thereto.

### Nuclear Import Proteins

In some embodiments, a TDSC or nucleic acid comprising dsDNA (e.g., as described herein) is capable of being imported into the nucleus, e.g., by a nuclear import protein (e.g., a nuclear import protein as listed in Table 3. In some embodiments, a TDSC or nucleic acid comprising dsDNA (e.g., as described herein) can be bound by a nuclear import protein (e.g., a nuclear import protein as listed in Table 3. In some embodiments, a TDSC or nucleic acid comprising dsDNA (e.g., as described herein) comprises a recognition sequence for a nuclear import protein (e.g., as listed in any single row of Table 3). In some embodiments, a TDSC or nucleic acid comprising dsDNA (e.g., as described herein) comprises a recognition sequence as listed in Table 3, or a nucleic acid sequence having at least 75%, 80%, 90%, 95%, 96%, 97%, 98%, or 99% identity thereto. In some embodiments, an exonuclease-resistant DNA end form (e.g., comprised in a TDSC or nucleic acid comprising dsDNA, e.g., as described herein) comprises a recognition sequence as listed in Table 3, or a nucleic acid sequence having at least 75%, 80%, 90%, 95%, 96%, 97%, 98%, or 99% identity thereto.

Exemplary import proteins include, e.g., basic helix-loop-helix (bHLH) proteins, heterogeneous nuclear ribonucleoprotein (hnRNP) isoforms, nuclear factor I (NFI) proteins, e.g., those listed in Table 3. In some embodiments, the bHLH protein comprises an acetylcholine receptor subunit, e.g., an alpha subunit, e.g., CHRNA1, CHRNA2, CHRNA3, CHRNA4, CHRNA5, or CHRNA7. In some embodiments, the acetylcholine receptor subunit comprises a gamma or epsilon subunit. In some embodiments, the import protein comprises a desmin. In some embodiments, the import protein comprises an hnRNP, e.g., an hnRNP A1, an hnRNP C, an hnRNP K, an hnRNP U. In some embodiments, the import protein comprises an importin. In some embodiments, the import protein comprises a myosin light chain. In some embodiments, the import protein comprises an NFI. In some embodiments, the import protein comprises an NFKB. In some embodiments, the import protein comprises a nucleoside diphosphate kinase, e.g., an NM23-H2. In some embodiments, the import protein comprises an Oct1. In some embodiments, the import protein comprises an Oct2.

In some embodiments, the import protein comprises a SRF. In some embodiments, the import protein comprises a TEF-1. In some embodiments, the import protein comprises an AP2. In some embodiments, the import protein comprises a troponin, e.g., a troponin I, e.g., a troponin 12. In some embodiments, the import protein comprises a TTF-1. In some embodiments, the import protein comprises a Ran binding protein, e.g., a RanBP3 or a RanBP1. In some embodiments, the import protein comprises a homeobox transcription factor, e.g., Chx10.

In some embodiments, the import factor specifically binds an E-box, a DTS (e.g., a SV40 DTS or a SMGA DTS), a promoter (e.g., a SP-C promoter or an htk promoter), a telomere, an ATTT motif, a cell cycle regulatory unit (CCRU), a CT3 sequence, an S/MAR, a topoisomerase II consensus sequence, an ARS consensus sequence, a 3NF, a viral ori (e.g., EBV oriP site).

TABLE 3
Exemplary nuclear import proteins and their corresponding recognition sequences

| Proteins that | | GenBank | Name of sequence | Corresponding DNA |
|---|---|---|---|---|
| facilitate | Protein | Accession # | recognized | recognition |
| nuclear entry | GeneID | for Protein | by protein | sequence |
| cardiac alpha- | 70 | NP_005150. | E-boxes | 5′-CANNTG-3′ |
| actin | | 1 | | |
| CHRNA1 | 1134 | NP_000070. | E-boxes | 5′-CANNTG-3′ |
| isoform b | | 1 | | |
| CHRNA1 | 1134 | NP_0010346 | E-boxes | 5′-CANNTG-3′ |
| isoform a | | 12.1 | | |
| CHRNA2 | 1135 | NP_000733. | E-boxes | 5′-CANNTG-3′ |
| isoform 1 | | 2 | | |
| CHRNA2 | 1135 | NP_0012693 | E-boxes | 5′-CANNTG-3′ |
| isoform 2 | | 84.1 | | |
| CHRNA2 | 1135 | NP_0013346 | E-boxes | 5′-CANNTG-3′ |
| isoform 3 | | 34.1 | | |
| CHRNA2 | 1135 | NP_0013346 | E-boxes | 5′-CANNTG-3′ |
| isoform 3 | | 35.1 | | |
| CHRNA2 | 1135 | NP_0013346 | E-boxes | 5′-CANNTG-3′ |
| isoform 4 | | 36.1 | | |
| CHRNA2 | 1135 | NP_0013346 | E-boxes | 5′-CANNTG-3′ |
| isoform 4 | | 37.1 | | |
| CHRNA3 | 1136 | NP_000734. | E-boxes | 5′-CANNTG-3′ |
| isoform 1 | | 2 | | |
| CHRNA3 | 1136 | NP_0011601 | E-boxes | 5′-CANNTG-3′ |
| isoform 2 | | 66.1 | | |
| CHRNA4 | 1137 | NP_000735. | E-boxes | 5′-CANNTG-3′ |
| isoform 1 | | 1 | | |
| CHRNA4 | 1137 | NP_0012435 | E-boxes | 5′-CANNTG-3′ |
| isoform 2 | | 02.1 | | |
| CHRNA5 | 1138 | NP_000736. | E-boxes | 5′-CANNTG-3′ |
| isoform 1 | | 2 | | |
| CHRNA5 | 1138 | NP_0012948 | E-boxes | 5′-CANNTG-3′ |
| isoform 2 | | 74.1 | | |
| CHRNA5 | 1138 | NP_0013821 | E-boxes | 5′-CANNTG-3′ |
| isoform 3 | | 00.1 | | |
| CHRNA5 | 1138 | NP_0013821 | E-boxes | 5′-CANNTG-3′ |
| isoform 4 | | 01.1 | | |
| CHRNA5 | 1138 | NP_ 0013821 | E-boxes | 5′-CANNTG-3′ |
| isoform 5 | | 02.1 | | |
| CHRNA5 | 1138 | NP_0013821 | E-boxes | 5′-CANNTG-3′ |
| isoform 6 | | 03.1 | | |
| CHRNA5 | 1138 | NP_0013821 | E-boxes | 5′-CANNTG-3′ |
| isoform 7 | | 04.1 | | |
| CHRNA7 | 1139 | NP_000737. | E-boxes | 5′-CANNTG-3′ |
| isoform 1 | | 1 | | |
| CHRNA7 | 1139 | NP_0011773 | E-boxes | 5′-CANNTG-3′ |
| isoform 2 | | 84.1 | | |
| CHRNE | 1145 | NP_000071. | E-boxes | 5′-CANNTG-3′ |
| | | 1 | | |
| CHRNG | 1146 | NP_005190. | E-boxes | 5′-CANNTG-3′ |
| | | 4 | | |
| M-creatine | 1158 | NP_001815. | E-boxes | 5′-CANNTG-3′ |
| kinase (MCK) | | 2 | | |
| desmin | 1674 | NP_0013696 | E-boxes | 5′-CANNTG-3′ |
| isoform 2 | | 37.1 | | |
| desmin | 1674 | NP_0013696 | E-boxes | 5′-CANNTG-3′ |
| isoform 3 | | 38.1 | | |
| desmin | 1674 | NP_0013696 | E-boxes | 5′-CANNTG-3′ |
| isoform 4 | | 39.1 | | |
| desmin | 1674 | NP_0013696 | E-boxes | 5′-CANNTG-3′ |
| isoform 5 | | 40.1 | | |
| desmin | 1674 | NP_0013696 | E-boxes | 5′-CANNTG-3′ |
| isoform 6 | | 41.1 | | |
| desmin | 1674 | NP_0013696 | E-boxes | 5′-CANNTG-3′ |
| isoform 7 | | 42.1 | | |
| desmin | 1674 | NP_001918. | E-boxes | 5′-CANNTG-3′ |
| isoform 1 | | 3 | | |
| AP1 (Fos) | 2353 | NP_005243. | SV40 DTS | 5′- |
| | | 1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| GATA-6 | 2627 | NP_005248. | SP-C promoter | 5′- |
| | | 2 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| hnRNP_A1 | 3178 | NP_002127. | dsDNA sequence from | 5′-GGCTGGTCTTGAACTCCTG (A/G) GCTCA (A/G) |
| isoform a | | 1 | human chromosome | GTGATCCTCC-3′ (SEQ ID NO: 109) |
| | | | band 11q13 (accession | |
| | | | number AC000353) | |
| hnRNP_A1 | 3178 | NP_112420. | dsDNA sequence from | 5′-GGCTGGTCTTGAACTCCTG (A/G) GCTCA (A/G) |
| isoform b | | 1 | human chromosome | GTGATCCTCC-3′ (SEQ ID NO: 109) |
| | | | band 11q13 (accession | |
| | | | number AC000353) | |
| hnRNP_A1 | 3178 | NP_002127. | Telomere (binds to | 5′-(TTAGGG)n-3′ |
| isoform a | | 1 | telomeric repeats) | |
| hnRNP_A1 | 3178 | NP_112420. | Telomere (binds to | 5′-(TTAGGG)n-3′ |
| isoform b | | 1 | telomeric repeats) | |
| hnRNP_A1 | 3178 | NP_002127. | human thymidine | 5′-TGCGGCCAAATCTCCCGCCAGGTCAGC-3′ (SEQ ID NO: |
| isoform a | | 1 | kinase (htk) promoter, | 110) |
| | | | ATTT sequence motif | |
| | | | contained within the | |
| | | | cell cycle regulatory | |
| | | | unit (CCRU) | |
| hnRNP_A1 | 3178 | NP_112420. | human thymidine | 5′-TGCGGCCAAATCTCCCGCCAGGTCAGC-3′ (SEQ ID NO: |
| isoform b | | 1 | kinase (htk) promoter, | 110) |
| | | | ATTT sequence motif | |
| | | | contained within the | |
| | | | cell cycle regulatory | |
| | | | unit (CCRU) | |
| hnRNP_A1 | 3178 | NP_002127. | human thymidine | 3′-ACGCCGGTTTAGAGGGCGGTCCAGTCG-5′ (SEQ ID NO: |
| isoform a | | 1 | kinase (htk) promoter, | 111) |
| | | | ATTT sequence motif | |
| | | | contained within the | |
| | | | cell cycle regulatory | |
| | | | unit (CCRU) | |
| hnRNP_A1 | 3178 | NP_112420. | human thymidine | 3′-ACGCCGGTTTAGAGGGCGGTCCAGTCG-5′ (SEQ ID NO: |
| isoform b | | 1 | kinase (htk) promoter, | 111) |
| | | | ATTT sequence motif | |
| | | | contained within the | |
| | | | cell cycle regulatory | |
| | | | unit (CCRU) | |
| hnRNP_C | 3183 | NP_0010709 | human thymidine | 5′-TGCGGCCAAATCTCCCGCCAGGTCAGC-3′ (SEQ ID NO: |
| isoform a | | 10.1 | kinase (htk) promoter, | 110) |
| | | | ATTT sequence motif | |
| | | | contained within the | |
| | | | cell cycle regulatory | |
| | | | unit (CCRU) | |
| hnRNP_C | 3183 | NP_0010709 | human thymidine | 5′-TGCGGCCAAATCTCCCGCCAGGTCAGC-3′ (SEQ ID NO: |
| isoform b | | 11.1 | kinase (htk) promoter, | 110) |
| | | | ATTT sequence motif | |
| | | | contained within the | |
| | | | cell cycle regulatory | |
| | | | unit (CCRU) | |
| hnRNP_C | 3183 | NP_004491. | human thymidine | 5′-TGCGGCCAAATCTCCCGCCAGGTCAGC-3′ (SEQ ID NO: |
| isoform b | | 2 | kinase (htk) promoter, | 110) |
| | | | ATTT sequence motif | |
| | | | contained within the | |
| | | | cell cycle regulatory | |
| | | | unit (CCRU) | |
| hnRNP_C | 3183 | NP_112604. | human thymidine | 5′-TGCGGCCAAATCTCCCGCCAGGTCAGC-3′ (SEQ ID NO: |
| isoform a | | 2 | kinase (htk) promoter, | 110 |
| | | | ATTT sequence motif | |
| | | | contained within the | |
| | | | cell cycle regulatory | |
| | | | unit (CCRU) | |
| hnRNP_C | 3183 | NP_0010709 | human thymidine | 3′-ACGCCGGTTTAGAGGGCGGTCCAGTCG-5′ (SEQ ID NO: |
| isoform a | | 10.1 | kinase (htk) promoter, | 111) |
| | | | ATTT sequence motif | |
| | | | contained within the | |
| | | | cell cycle regulatory | |
| | | | unit (CCRU) | |
| hnRNP_C | 3183 | NP_0010709 | human thymidine | 3′-ACGCCGGTTTAGAGGGCGGTCCAGTCG-5′ (SEQ ID NO: |
| isoform b | | 11.1 | kinase (htk) promoter, | 111) |
| | | | ATTT sequence motif | |
| | | | contained within the | |
| | | | cell cycle regulatory | |
| | | | unit (CCRU) | |
| hnRNP_C | 3183 | NP_004491. | human thymidine | 3′-ACGCCGGTTTAGAGGGCGGTCCAGTCG-5′ (SEQ ID NO: |
| isoform b | | 2 | kinase (htk) promoter, | 111) |
| | | | ATTT sequence motif | |
| | | | contained within the | |
| | | | cell cycle regulatory | |
| | | | unit (CCRU) | |
| hnRNP_C | 3183 | NP_112604. | human thymidine | 3′-ACGCCGGTTTAGAGGGCGGTCCAGTCG-5′ (SEQ ID NO: |
| isoform a | | 2 | kinase (htk) promoter, | 111) |
| | | | ATTT sequence motif | |
| | | | contained within the | |
| | | | cell cycle regulatory | |
| | | | unit (CCRU) | |
| hnRNP_K | 3190 | NP_0013051 | CT3 | 5′-AATTCTCCTCCCCACCTTCCCCACCCTCCCCA-3′ (SEQ ID |
| isoform c | | 15.1 | | NO: 112) |
| hnRNP_K | 3190 | NP_0013051 | CT3 | 5′-AATTCTCCTCCCCACCTTCCCCACCCTCCCCA-3′ (SEQ ID |
| isoform d | | 16.1 | | NO: 112) |
| hnRNP_K | 3190 | NP_0013051 | CT3 | 5′-AATTCTCCTCCCCACCTTCCCCACCCTCCCCA-3′ (SEQ ID |
| isoform b | | 17.1 | | NO: 112) |
| hnRNP_K | 3190 | NP_002131. | CT3 | 5′-AATTCTCCTCCCCACCTTCCCCACCCTCCCCA-3′ (SEQ ID |
| isoform a | | 2 | | NO: 112) |
| hnRNP_K | 3190 | NP_112552. | CT3 | 5′-AATTCTCCTCCCCACCTTCCCCACCCTCCCCA-3′ (SEQ ID |
| isoform b | | 1 | | NO: 112) |
| hnRNP_K | 3190 | NP_112553. | CT3 | 5′-AATTCTCCTCCCCACCTTCCCCACCCTCCCCA-3′ (SEQ ID |
| isoform a | | 1 | | NO: 112) |
| hnRNP_U (aka | 3192 | NP_004492. | S/MAR | 5′-TATATTT-3′ |
| SAF-A) | | 2 | | |
| isoform b | | | | |
| hnRNP_U (aka | 3192 | NP_114032. | S/MAR | 5′-TATATTT-3′ |
| SAF-A) | | 2 | | |
| isoform a | | | | |
| hnRNP_U (aka | 3192 | NP_004492. | S/MAR | 5′-(A/G) N (T/C) NNCNNG (T/C) NG |
| SAF-A) | | | | (G/T) TN (T/C) N (T/C)-3′ |
| isoform b | | 2 | | (SEQ ID NO: 113) |
| hnRNP_U (aka | 3192 | NP_114032. | S/MAR | 5′-(A/G) N (T/C) NNCNNG (T/C) NG |
| SAF-A) | | 2 | | (G/T) TN (T/C) N (T/C)-3′ |
| isoform a | | | | (SEQ ID NO: 113) |
| hnRNP_U (aka | 3192 | NP_004492. | S/MAR | 5′-(A/T) TTTAT (A/G) TTT (A/T)-3′ (SEQ ID NO: 114) |
| SAF-A) | | 2 | | |
| isoform b | | | | |
| hnRNP_U (aka | 3192 | NP_114032. | S/MAR | 5′-(A/T) TTTAT (A/G) TTT (A/T)-3′ (SEQ ID NO: 114) |
| SAF-A) | | 2 | | |
| isoform a | | | | |
| AP1 (Jun) | 3725 | NP_002219. | SV40 DTS | 5′- |
| | | 1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| Importin β1 | 3837 | XP_548162. | SMGA DTS | 5′- |
| | | 2 | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| myosin light | 4632 | NP_524144.1 | E-boxes | 5′-CANNTG-3′ |
| chain (MLC) | | | | |
| 1/3 isoform 1f | | | | |
| myosin light | 4632 | NP_524146.1 | E-boxes | 5′-CANNTG-3′ |
| chain (MLC) | | | | |
| 1/3 isoform 3f | | | | |
| NFI isoform 1 | 4782 | NP_001231931.1 | SP-C promoter | 5′- |
| | | | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| NFI isoform 3 | 4782 | NP_0012319 | SP-C promoter | 5′- |
| | | 33.1 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| NFI isoform 4 | 4782 | NP_0012319 | SP-C promoter | 5′- |
| | | 34.1 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| NFI isoform 5 | 4782 | NP_005588. | SP-C promoter | 5′- |
| | | 2 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| NFI isoform 2 | 4782 | NP_995315. | SP-C promoter | 5′- |
| | | 1 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| NFKB isoform | 4790 | NP_0011588 | 3NF | 5′- |
| 2 | | 84.1 | | CTGGGGACTTTCCAGCCTGGGGACTTTCCAGCTGGGACTTT |
| | | | | CCAGG-3′ (SEQ ID NO: 116) |
| NFKB isoform | 4790 | NP_0013061 | 3NF | 5′- |
| 2 | | 55.1 | | CTGGGGACTTTCCAGCCTGGGGACTTTCCAGCTGGGACTTT |
| | | | | CCAGG-3′ (SEQ ID NO: 116) |
| NFKB isoform | 4790 | NP_0013695 | 3NF | 5′- |
| 1 | | 54.1 | | CTGGGGACTTTCCAGCCTGGGGACTTTCCAGCTGGGACTTT |
| | | | | CCAGG-3′ (SEQ ID NO: 116) |
| NFKB isoform | 4790 | NP_0013695 | 3NF | 5′- |
| 1 | | 55.1 | | CTGGGGACTTTCCAGCCTGGGGACTTTCCAGCTGGGACTTT |
| | | | | CCAGG-3′ (SEQ ID NO: 116) |
| NFKB isoform | 4790 | NP_0013695 | 3NF | 5′- |
| 2 | | 56.1 | | CTGGGGACTTTCCAGCCTGGGGACTTTCCAGCTGGGACTTT |
| | | | | CCAGG-3′ (SEQ ID NO: 116) |
| NFKB isoform | 4790 | NP_0013695 | 3NF | 5′- |
| 3 | | 57.1 | | CTGGGGACTTTCCAGCCTGGGGACTTTCCAGCTGGGACTTT |
| | | | | CCAGG-3′ (SEQ ID NO: 116) |
| NFKB isoform | 4790 | NP_003989. | 3NF | 5′- |
| 1 | | 2 | | CTGGGGACTTTCCAGCCTGGGGACTTTCCAGCTGGGACTTT |
| | | | | CCAGG-3′ (SEQ ID NO: 116) |
| NFKB isoform | 4790 | NP_0011588 | SV40 DTS | 5′- |
| 2 | | 84.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_0013061 | SV40 DTS | 5′- |
| 2 | | 55.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_0013695 | SV40 DTS | 5′- |
| 1 | | 54.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_0013695 | SV40 DTS | 5′- |
| 1 | | 55.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_0013695 | SV40 DTS | 5′- |
| 2 | | 56.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_0013695 | SV40 DTS | 5′- |
| 3 | | 57.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_003989. | SV40 DTS | 5′- |
| 1 | | 2 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NFKB isoform | 4790 | NP_0011588 | A fragment consisting | 5′- |
| 2 | | 84.1 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC- | |
| | | | 3′(SEQ ID NO: 117) | |
| NFKB isoform | 4790 | NP_0013061 | A fragment consisting | 5′- |
| 2 | | 55.1 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC-3′ | |
| | | | (SEQ ID NO: 117) | |
| NFKB isoform | 4790 | NP_0013695 | A fragment consisting | 5′- |
| 1 | | 54.1 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC-3′ | |
| | | | (SEQ ID NO: 117) | |
| NFKB isoform | 4790 | NP_0013695 | A fragment consisting | 5′- |
| 1 | | 55.1 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC-3′ | |
| | | | (SEQ ID NO: 117) | |
| NFKB isoform | 4790 | NP_0013695 | A fragment consisting | 5′- |
| 2 | | 56.1 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC-3′ | |
| | | | (SEQ ID NO: 117) | |
| NFKB isoform | 4790 | NP_0013695 | A fragment consisting | 5′- |
| 3 | | 57.1 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC-3′ | |
| | | | (SEQ ID NO: 117) | |
| NFKB isoform | 4790 | NP_003989. | A fragment consisting | 5′- |
| 1 | | 2 | of five tandem repeats | GGGGACTTTCCGGGGACTTTCCGGGGACTTTCCGGGGACTT |
| | | | of the IgK KB motif 5′- | TCCGGGGACTTTCC-3′ (SEQ ID NO: 118) |
| | | | GGGGACTTTCC-3′ | |
| | | | (SEQ ID NO: 117) | |
| NM23-H2 | 4831 | NP_0010181 | SV40 DTS | 5′- |
| isoform a | | 47.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NM23-H2 | 4831 | NP_0010181 | SV40 DTS | 5′- |
| isoform a | | 48.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NM23-H2 | 4831 | NP_0010181 | SV40 DTS | 5′- |
| isoform a | | 49.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NM23-H2 | 4831 | NP_0011856 | SV40 DTS | 5′- |
| isoform b | | 11.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| NM23-H2 | 4831 | NP_002503. | SV40 DTS | 5′- |
| isoform a | | 1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| Oct2 | 5452 | NP_0011939 | SV40 DTS | 5′- |
| (POU2F2) | | 54.1 | | GGTGTGGAAAGTCCCCAGGCTCCCCAGCAGGCAGAAGTAT |
| isoform 1 | | | | GCAAAGCATGCATCTCAATTAGTCAGCAACCA-3′ (SEQ ID |
| | | | | NO: 107) |
| Oct2 | 5452 | NP_0011939 | SV40 DTS | 5′- |
| (POU2F2) | | 55.1 | | GGTGTGGAAAGTCCCCAGGCTCCCCAGCAGGCAGAAGTAT |
| isoform 3 | | | | GCAAAGCATGCATCTCAATTAGTCAGCAACCA-3′ (SEQ ID |
| | | | | NO: 107) |
| Oct2 | 5452 | NP_0012349 | SV40 DTS | 5′- |
| (POU2F2) | | 23.1 | | GGTGTGGAAAGTCCCCAGGCTCCCCAGCAGGCAGAAGTAT |
| isoform 4 | | | | GCAAAGCATGCATCTCAATTAGTCAGCAACCA-3′ (SEQ ID |
| | | | | NO: 107) |
| Oct2 | 5452 | NP_0013808 | SV40 DTS | 5′- |
| (POU2F2) | | 63.1 | | GGTGTGGAAAGTCCCCAGGCTCCCCAGCAGGCAGAAGTAT |
| isoform 5 | | | | GCAAAGCATGCATCTCAATTAGTCAGCAACCA-3′ (SEQ ID |
| | | | | NO: 107) |
| Oct2 | 5452 | NP_0013808 | SV40 DTS | 5′- |
| (POU2F2) | | 64.1 | | GGTGTGGAAAGTCCCCAGGCTCCCCAGCAGGCAGAAGTAT |
| isoform 6 | | | | GCAAAGCATGCATCTCAATTAGTCAGCAACCA-3′ (SEQ ID |
| | | | | NO: 107) |
| Oct2 | 5452 | NP_0013808 | SV40 DTS | 5′- |
| (POU2F2) | | 65.1 | | GGTGTGGAAAGTCCCCAGGCTCCCCAGCAGGCAGAAGTAT |
| isoform 7 | | | | GCAAAGCATGCATCTCAATTAGTCAGCAACCA-3′ (SEQ ID |
| | | | | NO: 107) |
| Oct2 | 5452 | NP_0013813 | SV40 DTS | 5′- |
| (POU2F2) | | 05.1 | | GGTGTGGAAAGTCCCCAGGCTCCCCAGCAGGCAGAAGTAT |
| isoform 8 | | | | GCAAAGCATGCATCTCAATTAGTCAGCAACCA-3′ (SEQ ID |
| | | | | NO: 107) |
| Oct2 | 5452 | NP_0013813 | SV40 DTS | 5′- |
| (POU2F2) | | 06.1 | | GGTGTGGAAAGTCCCCAGGCTCCCCAGCAGGCAGAAGTAT |
| isoform 9 | | | | GCAAAGCATGCATCTCAATTAGTCAGCAACCA-3′ (SEQ ID |
| | | | | NO: 107) |
| Oct2 | 5452 | NP_0013813 | SV40 DTS | 5′- |
| (POU2F2) | | 07.1 | | GGTGTGGAAAGTCCCCAGGCTCCCCAGCAGGCAGAAGTAT |
| isoform 10 | | | | GCAAAGCATGCATCTCAATTAGTCAGCAACCA-3′ (SEQ ID |
| | | | | NO: 107) |
| Oct2 | 5452 | NP_002689. | SV40 DTS | 5′- |
| (POU2F2) | | 1 | | GGTGTGGAAAGTCCCCAGGCTCCCCAGCAGGCAGAAGTAT |
| isoform 2 | | | | GCAAAGCATGCATCTCAATTAGTCAGCAACCA-3′ (SEQ ID |
| | | | | NO: 107) |
| RanBP1 | 5902 | XP_514990. | SMGA DTS | 5′- |
| | | 2 | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| Oct1 | 5451 | NP_0011857 | SV40 DTS | 5′- |
| (POU2F1) | | 12.1 | | GGTGTGGAAAGTCCCCAGGCTCCCCAGCAGGCAGAAGTAT |
| isoform 2 | | | | GCAAAGCATGCATCTCAATTAGTCAGCAACCA-3′ (SEQ ID |
| | | | | NO: 107) |
| Oct1 | 5451 | NP_0011857 | SV40 DTS | 5′- |
| (POU2F1) | | 15.1 | | GGTGTGGAAAGTCCCCAGGCTCCCCAGCAGGCAGAAGTAT |
| isoform 3 | | | | GCAAAGCATGCATCTCAATTAGTCAGCAACCA-3′ (SEQ ID |
| | | | | NO: 107) |
| Oct1 | 5451 | NP_0013527 | SV40 DTS | 5′- |
| (POU2F1) | | 77.1 | | GGTGTGGAAAGTCCCCAGGCTCCCCAGCAGGCAGAAGTAT |
| isoform 4 | | | | GCAAAGCATGCATCTCAATTAGTCAGCAACCA-3′ (SEQ ID |
| | | | | NO: 107) |
| Oct1 | 5451 | NP_0013527 | SV40 DTS | 5′- |
| (POU2F1) | | 78.1 | | GGTGTGGAAAGTCCCCAGGCTCCCCAGCAGGCAGAAGTAT |
| isoform 4 | | | | GCAAAGCATGCATCTCAATTAGTCAGCAACCA-3′ (SEQ ID |
| | | | | NO: 107) |
| Oct1 | 5451 | NP_002688. | SV40 DTS | 5′- |
| (POU2F1) | | 3 | | GGTGTGGAAAGTCCCCAGGCTCCCCAGCAGGCAGAAGTAT |
| isoform 1 | | | | GCAAAGCATGCATCTCAATTAGTCAGCAACCA-3′ (SEQ ID |
| | | | | NO: 107) |
| SRF isoform 2 | 6722 | NP_0012789 | SMGA DTS | 5′- |
| | | 30.1 | | TTCAAATGATAACTCACTCTACCCACCCCCCTTCCCTACCCC |
| | | | | CAAGGCGATTTATTGAAAAAACCACCTTATATGGTAATATT |
| | | | | GCTAACACACCGTCAGCTGGCCTTTTTAGGGACTTTGTTTA |
| | | | | AAGAAGATCCGCCTCTGGGGTTTTATATTGCTCTGGTATTC |
| | | | | ATGCCAAAGAC-3′ (SEQ ID NO: 119) |
| SRF isoform 1 | 6722 | NP_003122. | SMGA DTS | 5′- |
| | | 1 | | TTCAAATGATAACTCACTCTACCCACCCCCCTTCCCTACCCC |
| | | | | CAAGGCGATTTATTGAAAAAACCACCTTATATGGTAATATT |
| | | | | GCTAACACACCGTCAGCTGGCCTTTTTAGGGACTTTGTTTA |
| | | | | AAGAAGATCCGCCTCTGGGGTTTTATATTGCTCTGGTATTC |
| | | | | ATGCCAAAGAC-3′ (SEQ ID NO: 119) |
| TEF-1 isoform | 7008 | NP_0011388 | SV40 DTS | 5′- |
| 2 | | 70.1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| TEF-1 isoform | 7008 | NP_003207. | SV40 DTS | 5′- |
| 1 | | 1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| AP2 | 7020 | NP_0010274 | SV40 DTS | 5′- |
| (TFAP2A) | | 51.1 | | GGTGTGGAAAGTCCCCAGGCTCCCCAGCAGGCAGAAGTAT |
| isoform b | | | | GCAAAGCATGCATCTCAATTAGTCAGCAACCA-3′ (SEQ ID |
| | | | | NO: 107) |
| AP2 | 7020 | NP_0010358 | SV40 DTS | 5′- |
| (TFAP2A) | | 90.1 | | GGTGTGGAAAGTCCCCAGGCTCCCCAGCAGGCAGAAGTAT |
| isoform c | | | | GCAAAGCATGCATCTCAATTAGTCAGCAACCA-3′ (SEQ ID |
| | | | | NO: 107) |
| AP2 | 7020 | NP_0013589 | SV40 DTS | 5′- |
| (TFAP2A) | | 95.1 | | GGTGTGGAAAGTCCCCAGGCTCCCCAGCAGGCAGAAGTAT |
| isoform a | | | | GCAAAGCATGCATCTCAATTAGTCAGCAACCA-3′ (SEQ ID |
| | | | | NO: 107) |
| AP2 | 7021 | NP_003212. | SV40 DTS | 5′- |
| (TFAP2B) | | 2 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| AP2 | 7022 | NP_003213. | SV40 DTS | 5′- |
| (TFAP2C) | | 1 | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| troponin I 1 | 7135 | NP_003272. | E-boxes | 5′-CANNTG-3′ |
| | | 3 | | |
| troponin I 2 - | 7136 | NP_0011393 | E-boxes | 5′-CANNTG-3′ |
| isoform 1 | | 01.1 | | |
| troponin I 2 - | 7136 | NP_0011393 | E-boxes | 5′-CANNTG-3′ |
| isoform 2 | | 13.1 | | |
| troponin I 2 - | 7136 | NP_003273. | E-boxes | 5′-CANNTG-3′ |
| isoform 1 | | 1 | | |
| troponin I 3 | 7137 | NP_000354.4 | E-boxes | 5′-CANNTG-3′ |
| TTF-1 isoform | 7270 | NP_0011922 | SP-C promoter | 5′- |
| 2 | | 25.1 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| TTF-1 isoform | 7270 | NP_031370. | SP-C promoter | 5′- |
| 1 | | 2 | | CAGGGCAGCAGGGGCAGGTGCCAGCAAGGAAGGCAGGCA |
| | | | | CGCCAGGAAGACACCCATGGTGAGAAGTGCAGATGGCCCG |
| | | | | AGGGCAAGTTTGCTCAACTCACCCAGGTTTGCTCTTGCTGG |
| | | | | GGCCAAGAGGACTCATGTGCCAGGGCCAAGGGCCCTTGGG |
| | | | | GGCTCTCACAGGGGGCTTATCTGGGCTTCGGTTCTGGAGGG |
| | | | | CCAGGAACAAACAGGCTTCAAAGCCAAGGGCTTGGCTGGC |
| | | | | ACACAGGGGGCTTGGTCCTTCACCTCTGTCCCCTCTCCCTAC |
| | | | | GGACACATATAAGACCCTGGTCACACCTGGGAGAGGAGGA |
| | | | | GAGGAGAGCATAG-3′ (SEQ ID NO: 108) |
| H2B | 8349 | NP_003519.1 | SV40 DTS | 5′- |
| | | | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| RanBP3 | 8498 | NP_001287794.1 | SMGA DTS | 5′- |
| isoform 4 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | NP_003615.2 | SMGA DTS | 5′- |
| isoform a | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | NP_015559.2 | SMGA DTS | 5′- |
| isoform b | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | NP_015561.1 | SMGA DTS | 5′- |
| isoform d | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | XP_006722991.1 | SMGA DTS | 5′- |
| isoform X1 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | XP_006722992.1 | SMGA DTS | 5′- |
| isoform X1 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | XP_011526695.1 | SMGA DTS | 5′- |
| isoform X1 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | XP_011526694.1 | SMGA DTS | 5′- |
| isoform X1 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | XP_011526696.1 | SMGA DTS | 5′- |
| isoform X1 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| RanBP3 | 8498 | XP_024307517.1 | SMGA DTS | 5′- |
| isoform X1 | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| importin 7 | 10527 | NP_006382.1 | SMGA DTS | 5′- |
| | | | | AGGCAGACCCAGGGGCCGCATGCAGCAGGGCCTGAGGAGG |
| | | | | GAGGTGTGGACGGAGGAGGCCCGCTGCCATTCTTGGTATG |
| | | | | GTTCTCACTCCAGGAGCACAGCTGCATCTGGTCTCACTCTG |
| | | | | GGCAGCTTATAAGGCCTGGTGTGAGTTTTGTTTATGCAAGT |
| | | | | GCAGCATAAAAGGAACAAATCTACCAGCACCGGGGCTGTT |
| | | | | GCCACTGAGTCCTTTTGCATACATTTTTCAAATGATAACTCA |
| | | | | CTCTACCCACCCCCCTTCCCTACCCCCAAGGCGATTTATTGA |
| | | | | AAAAACCACCTTATATGGTAATATTGCTAACACACCGTCAG |
| | | | | CTGGCCTTTTTAGGGACTTTGTTTAAAGAAGATCCGCCTCT |
| | | | | GGGGTTTTATATTGCTCTGGTATTCATGCCAAAGAC-3′ (SEQ |
| | | | | ID NO: 115) |
| Chx10 | 338917 | NP_878314.1 | SV40 DTS | 5′- |
| | | | | GGTGTGGAAAGTCCCCAGGCTCCCCAGCAGGCAGAAGTAT |
| | | | | GCAAAGCATGCATCTCAATTAGTCAGCAACCA-3′ (SEQ ID |
| | | | | NO: 107) |
| EBNA-1 | 3783774 | YP_401677.1 | EBV oriP site | 5′- |
| | | | | CATGCAGGAAAAGGACAAGCAGCGAAAATTCACGCCCCCT |
| | | | | TGGGAGGTGGCGGCATATGCAAAGGATAGCACTCCCACTC |
| | | | | TACTACTGGGTATCATATGCTGACTGTATATGCATGAGGAT |
| | | | | AGCATATGCTACCCGGATACAGATTAGGATAGCATATACTA |
| | | | | CCCAGATATAGATTAGGATAGCATATGCTACCCAGATATAG |
| | | | | ATTAGGATAGCCTATGCTACCCAGATATAAATTAGGATAGC |
| | | | | ATATACTACCCAGATATAGATTAGGATAGCATATGCTACCC |
| | | | | AGATATAGATTAGGATAGCCTATGCTACCCAGATATAGATT |
| | | | | AGGATAGCATATGCTACCCAGATATAGATTAGGATAGCAT |
| | | | | ATGCTATCCAGATATTTGGGTAGTATATGCTACCCAGATAT |
| | | | | AAATTAGGATAGCATATACTACCCTAATCTCTATTAGGATA |
| | | | | GCATATGCTACCCGGATACAGATTAGGATAGCATATACTAC |
| | | | | CCAGATATAGATTAGGATAGCATATGCTACCCAGATATAGA |
| | | | | TTAGGATAGCCTATGCTACCCAGATATAAATTAGGATAGCA |
| | | | | TATACTACCCAGATATAGATTAGGATAGCATATGCTACCCA |
| | | | | GATATAGATTAGGATAGCCTATGCTACCCAGATATAGATTA |
| | | | | GGATAGCATATGCTATCCAGATATTTGGGTAGTATATGCTA |
| | | | | CCCATGGCAACATTAGCCCACCGTGCTCTCAGCGACCTCGT |
| | | | | GAATATGAGGACCAACAACCCTGTGCTTGGCGCTCAGGCG |
| | | | | CAAGTGTGTGTAATTTGTCCTCCAGATCGCAGCAATCGCGC |
| | | | | CCCTATCTTGGCCCGCCCACCTACTTATGCAGGTATTCCCCG |
| | | | | GGGTGCCATTAGTGGTTTTGTGGGCAAGTGGTTTGACCGCA |
| | | | | GTGGTTAGCGGGGTTACAATCAGCCAAGTTATTACACCCTT |
| | | | | ATTTTACAGTCCAAAACCGCAGGGCGGCGTGTGGGGGCTG |
| | | | | ACGCGTGCCCCCACTCCACAATTTCAAAAAAAAGAGTGGC |
| | | | | CACTTGTCTTTGTTTATGGGCCCCATTGGCGTGGAGCCCCGT |
| | | | | TTAATTTTCGGGGGTGTTAGAGACAACCAGTGGAGTCCGCT |
| | | | | GCTGTCGGCGTCCACTCTCTTTCCCCTTGTTACAAATAGAGT |
| | | | | GTAACAACATGGTTCACCTGTCTTGGTCCCTGCCTGGGACA |
| | | | | CATCTTAATAACCCCAGTATCATATTGCACTAGGATTATGT |
| | | | | GTTGCCCATAGCCATAAATTCGTGTGAGATGGACATCCAGT |
| | | | | CTTTACGGCTTGTCCCCACCCCATGGATTTCTATTGTTAAAG |
| | | | | ATATTCAGAATGTTTCATTCCTACACTAGTATTTATTGCCCA |
| | | | | AGGGGTTTGTGAGGGTTATATTGGTGTCATAGCACAATGCC |
| | | | | ACCACTGAACCCCCCGTCCAAATTTTATTCTGGGGGCGTCA |
| | | | | CCTGAAACCTTGTTTTCGAGCACCTCACATACACCTTACTGT |
| | | | | TCACAACTCAGCAGTTATTCTATTAGCTAAACGAAGGAGAA |
| | | | | TGAAGAAGCAGGCGAAGATTCAGGAGAGTTCACTGCCCGC |
| | | | | TCCTTGATCTTCAGCCACTGCCCTTGTGACTAAAATGGTTCA |
| | | | | CTACCCTCGTGGAATCCTGACCCCATGTAAATAAAACCGTG |
| | | | | ACAGCTCATGGGGTGGGAGATATCGCTGTTCCTTAGGACCC |
| | | | | TTTTACTAACCCTAATTCGATAGCATATGCTTCCCGTTGGGT |
| | | | | AACATATGCTATTGAATTAGGGTTAGTCTGGATAGTATATA |
| | | | | CTACTACCCGGGAAGCATATGCTACCCGTTTAGGGTTAACA |
| | | | | AGGGGGCCTTATAAACACTATTGCTAATGCCCTCTTGAGGG |
| | | | | TCCGCTTATCGGTAGCTACACAGGCCCCTCTGATTGACGTT |
| | | | | GGTGTAGCCTCCCGTAGTCTTCCTGGGCCCCTGGGAGGTAC |
| | | | | ATGTCCCCCAGCATTGGTGTAAGAGCTTCAGCCAAGAGTTA |
| | | | | CACATAAAGGCAATGTTGTGTTGCAGTCCACAGACTGCAAA |
| | | | | GTCTGCTCCAGGATGAAAGCCACTCAGTGTTGGCAAATGTG |
| | | | | CACATCCATTTATAAGGATGTCAACTACAGTCAGAGAACCC |
| | | | | CTTTGTGTTTGGTCCCCCCCCGTGTCACATGTGGAACAGGG |
| | | | | CCCAGTTGGCAAGTTGTACCAACCAACTGAAGGGATTACAT |
| | | | | GCACTGCCCCGC-3′ (SEQ ID NO: 120) |
| NKX3-1/3-2 | 4824 | NP_001243268.1 | SMGA DTS | 5′- |
| isoform 2 | | | | TTCAAATGATAACTCACTCTACCCACCCCCCTTCCCTACCCC |
| | | | | CAAGGCGATTTATTGAAAAAACCACCTTATATGGTAATATT |
| | | | | GCTAACACACCGTCAGCTGGCCTTTTTAGGGACTTTGTTTA |
| | | | | AAGAAGATCCGCCTCTGGGGTTTTATATTGCTCTGGTATTC |
| | | | | ATGCCAAAGAC-3′ (SEQ ID NO: 119) |
| NKX3-1/3-2 | 4824 | NP_006158.2 | SMGA DTS | 5′- |
| isoform 1 | | | | TTCAAATGATAACTCACTCTACCCACCCCCCTTCCCTACCCC |
| | | | | CAAGGCGATTTATTGAAAAAACCACCTTATATGGTAATATT |
| | | | | GCTAACACACCGTCAGCTGGCCTTTTTAGGGACTTTGTTTA |
| | | | | AAGAAGATCCGCCTCTGGGGTTTTATATTGCTCTGGTATTC |
| | | | | ATGCCAAAGAC-3′ (SEQ ID NO: 119) |

### Maintenance Sequence

A TDSC or nucleic acid comprising dsDNA disclosed herein may include a maintenance sequence that supports or enables sustained gene expression through successive rounds of cell division and/or progenitor differentiation in a host cell for a TDSC of the invention. In embodiments, a maintenance sequence is a nuclear scaffold/matrix attachment region (S/MAR). S/MAR elements are diverse, AT-rich sequences ranging from 60-500 bp that are conserved across species, thought to anchor chromatin to nuclear matrix proteins during interphase (Bode et al. 2003. Chromosome Res 11, 435-445). An S/MAR can be incorporated into a TDSC described herein to facilitate long-term transgene expression and extra-chromosomal maintenance. In one embodiment, the maintenance sequence is human interferon-beta MAR (5′tataattcactggaatttttttgtgtgtatggtatgacatatgggttcccttttattttttacatataaatatatttccctgtttttctaaaaaagaaaa agatcatcattttcccattgtaaaatgccatatttttttcataggtcacttacata-3′ (SEQ ID NO: 39)), or a functional sequence having at least 80%, 90%, 95%, or 98% identity thereto. In embodiments, S/MARs useful in the constructs described herein can be found by searching the MARome at http://bioinfo.net.in/MARome, described also by Narwade et al. 2019. Nucleic Acids Research. Volume 47, Issue 14:7247-7261.

In embodiments, a TDSC described herein is capable of replicating in a mammalian cell, e.g., human cell. In some embodiments, a TDSC described herein is maintained in a host cell, tissue or subject through at least one cell division. For example, a TDSC described herein is maintained in a host cell, tissue or subject through at least 2, 3, 4, 5, 6, 7, 8, 10, 15, 20, 40, 50 or more cell divisions. In vitro, cell division may be tracked by flow cytometry or microscopy. In vivo, cell division may be tracked by intravital microscopy.

### Other Elements

A TDSC or nucleic acid comprising dsDNA disclosed herein may also include other control elements operably linked to the effector sequence, e.g., the sequence encoding an effector, in a manner which permits its transport, localization, transcription, translation and/or expression in a target cell, or which promotes its degradation or repression of expression in a non-target cell. As used herein, “operably linked” sequences include both expression control sequences that are contiguous with the sequence encoding the effector and expression control sequences that act in trans or at a distance to control the sequence encoding the effector. The precise nature of regulatory sequences needed for gene expression in host cells may vary between species, tissues or cell types, but in general may include, as necessary, 5′ non-transcribed and 5′ non-translated sequences involved with the initiation of transcription and translation respectively, such as a TATA box, capping sequence, CAAT sequence, enhancer elements and the like. Regulatory sequences may also include enhancer sequences or upstream activator sequences as desired. The constructs described herein may optionally include 5′ leader or signal sequences.

### Chemically Modified Nucleotides

The TDSC or nucleic acid comprising dsDNA and compositions described herein may have chemical modifications of the nucleobases, sugars, and/or the phosphate backbone (e.g., as shown in FIGS. 1A-2). While not wishing to be bound by theory, such modifications can be useful for protecting a DNA from degradation (e.g., from exonucleases) or from the immune system of a host tissue or subject. In general, a chemically modified nucleotide has the same base-pairing specificity as the unmodified nucleotide, e.g., a chemically modified adenine “A” can base-pair with thymine “T”. One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, chemical modifications (e.g., one or more modifications) are present in each of the sugar and the internucleoside linkage.

In some embodiments, the TDSC comprises at least one chemical modification. Suitable modifications are described by Sood et al. 2019. DNAmod: the DNA modification database. J Cheminform 11, 30. DNAmod is an open-source database (https://dnamod.hoffmanlab.org) that catalogues chemically modified nucleotides and provides a single source to learn about their properties. DNAmod provides a web interface to easily browse and search through these modifications. The database annotates the chemical properties and structures of all curated chemically modified DNA bases, and a much larger list of candidate chemical entities. DNAmod includes manual annotations of available sequencing methods, descriptions of their occurrence in nature, and provides existing and suggested nomenclature. Examples of chemical modifications to DNA useful in the methods described herein include, e.g., N6-Methyladenosine (m6A, 6 mA); 5-formylcytosine (5-formyl-2′-deoxycytosine, 5fC, f5C); 5-carboxylcytosine (5-carboxyl-2′-deoxycytosine, 5-carboxycytosine, ca5C, 5caC); 5-hydroxymethylcytosine (5-hydroxymethyl-2′-deoxycytosine, 5hmC, hm5C); 5-methyldeoxycytosine (5-methylcytosine; 5-methyl-2′-deoxycytosine; m5dC; 5mC, m5C); 5′-methylcytosine; 3-methylcytosine (m3C); 2′-fluoro-2′deoxynucleoside; 5-glucosylmethylcytosine; 5-methyl pyrimidine; 8-oxoguanine (8-oxoG); phosphorothioate; S and R phsophorothioate linkages; methylthymine; N3′-P5′ Phosphoroamidate (NP); cyclohexane nucleic acid (CeNA); tricyclo-DNA (tcDNA). See, e.g., Pu et al. 2020. An in-vitro DNA phosphorothioate modification reaction. Mol Microbiol. 113:452-463; Zheng & Sheng. 2021. Synthesis of N4-methylcytidine (m4C) and N4,N4-dimethylcytidine (m42C) modified RNA. Current Protocols, 1, e248; Ohkubo et al. 2021. Chemical synthesis of modified oligonucleotides containing 5′-amino-5′-deoxy-5′-hydroxymethylthymidine residues. Current Protocols, 1, e70; Bao & Xu. 2021. Observation of Z-DNA structure via the synthesis of oligonucleotide DNA containing 8-trifluoromethyl-2-deoxyguanosine. Current Protocols, 1, e28; Skakuj et al. 2020. Automated synthesis and purification of guanidine-backbone oligonucleotides. Current Protocols in Nucleic Acid Chemistry, 81, e110.

In some embodiments, a TDSC as described herein may comprise a phosphorothioate-modified nucleotide. In some embodiments, a DNA end form (e.g., an exonuclease-resistant DNA end form) as described herein may comprise a phosphorothioate-modified nucleotide. In some embodiments, the TDSCs described herein may include S and R phosphorothioate modified nucleotide linkages. In one embodiment, the phosphorothioate linkages are made according to Iwamoto et al, 2017, Nature Biotechnology, Volume 35:845-851. Briefly, monomers of nucleoside 3′-oxazaphospholidine derivates undergo stereocontrolled oligonucleotide synthesis with iterative capping and sulfurization to create stereocontrolled phosphorothioate linkages. The final sample is analyzed by reverse-phase high-performance liquid chromatography (RP-HPLC) and Ultraperformance liquid chromatography mass spectrometry (UPLC/MS) to determine stereochemistry of the modification. Nucleic acids containing phosphorothioate linkages are also commercially available.

In some embodiments, the TDSCs described herein may include boranophosphate modified nucleotides, e.g., following the methods in Sergueev and Shaw, 1998, J Am Chem Soc, Volume 120, Issue 37:9417-9427. Briefly, H-phosphonate chain elongation is followed by boronation to substitute a borano group for a nonbridging oxygen in the phosphate backbone. The final sample is purified and analyzed by RP-HPLC to determine stereochemistry of the modification. Boranophosphate modified nucleotides are also commercially available.

In some embodiments, the TDSCs described herein may include 5-methylcytosine modified nucleotides, e.g., made following the methods in Lin et al, 2002, Mol Cell Biol, Volume 22, Issue 3:704-723. Briefly, cytosine or the sequence containing cytosine is incubated with glutathione S-transferase fusion of wild-type Dnmt3a (GST-3a) protein using unlabeled S-adenosylmethionine (AdoMet). The nucleotides are purified and analyzed by HPLC to determine that the nucleotides are methylated at the correct position. 5-methylcytosine modified nucleotides are also available commercially.

In some embodiments, the TDSCs described herein may include 7-methylguanine modified nucleotides. In one embodiment, 7-methylguanine modified nucleotides are made following the methods in Jones and Robins, 1963, Purine nucleosides. III. Methylation studies of certain naturally occurring purine nucleosides, J Am Chem Soc, Volume 85:193. Briefly, 2′-deoxyguanosine in dimethyl sulfoxide is treated with methyl iodide. The nucleotides are purified and analyzed by HPLC to determine that the nucleotides are methylated at the correct position. In another embodiment, 7-methylguanine modified nucleotides are made according to the methods described in Hendler et al, 1970, Volume 9, Issue 21:4141:4153, and Kore and Parmar, 2006, Biochemistry, Volume 25, Issue 3:337-340. Briefly, instead of guanosine 5′-diphosphate, guanine 5′-diphosphate in water is added to dimethyl sulfate to yield 7-methyl GDP. The nucleotides are purified and analyzed by HPLC to determine that the nucleotides are methylated at the correct position. 7-methylguanine modified nucleotides are also available commercially.

In some embodiments, a TDSC described herein comprises methylation at one or more CpG or GpC dinucleotide. In some embodiments, a TDSC described herein comprises a methylation introduced by an AluI methyltransferase. In some embodiments, a TDSC described herein comprises a methylation introduced by a BamHI methyltransferase. In some embodiments, a TDSC described herein comprises a methylation introduced by a CpG methyltransferase (M.Sssl). In some embodiments, a TDSC described herein comprises a methylation introduced by a dam methyltransferase. In some embodiments, a TDSC described herein comprises a methylation introduced by an EcoGII methyltransferase. In some embodiments, a TDSC described herein comprises a methylation introduced by an EcoRI methyltransferase. In some embodiments, a TDSC described herein comprises a methylation introduced by a GpC methyltransferase (M.CviPI). In some embodiments, a TDSC described herein comprises a methylation introduced by an HaeIII methyltransferase. In some embodiments, a TDSC described herein comprises a methylation introduced by an HhaI methyltransferase. In some embodiments, a TDSC described herein comprises a methylation introduced by an HpaII methyltransferase. In some embodiments, a TDSC described herein comprises a methylation introduced by a MspI methyltransferase. In some embodiments, a TDSC described herein comprises a methylation introduced by a TaqI methyltransferase. In some embodiments, a method described herein comprises contacting a dsDNA with an AluI methyltransferase, a BamHI methyltransferase, M.Sssl, a dam methyltransferase, an EcoGII methyltransferase, an EcoRI methyltransferase, M.CviPI, an HaeIII methyltransferase, an HhaI methyltransferase, an HpaII methyltransferase, a MspI methyltransferase, or a TaqI methyltransferase.

In some embodiments, a TDSC described herein comprises a carboxyl modification or a formyl modification.

In embodiments, a TDSC described herein, or one strand (e.g., the sense strand or the antisense strand) of the TDSC, comprises between 1-100% chemically modified nucleotides, between 1%-90% chemically modified nucleotides, between 1%-80% chemically modified nucleotides, between 1%-70% chemically modified nucleotides, between 1%-60% chemically modified nucleotides, between 1%-50% chemically modified nucleotides, between 1%-40% chemically modified nucleotides, between 1%-30% chemically modified nucleotides, between 1%-20% chemically modified nucleotides, between 1%-15% chemically modified nucleotides, between 1%-10% chemically modified nucleotides, between 20%-90% chemically modified nucleotides, between 20%-80% chemically modified nucleotides. In embodiments, a TDSC described herein, or one strand (e.g., the sense strand or the antisense strand) of the TDSC, comprises at least 1% chemically modified nucleotides, at least 5% chemically modified nucleotides; at least 10% chemically modified nucleotides; at least 15% chemically modified nucleotides; at least 20% chemically modified nucleotides; at least 25% chemically modified nucleotides; at least 30% chemically modified nucleotides; at least 40% chemically modified nucleotides; at least 50% chemically modified nucleotides; at least 60% chemically modified nucleotides; at least 70% chemically modified nucleotides; at least 80% chemically modified nucleotides; at least 85% chemically modified nucleotides; at least 90% chemically modified nucleotides; at least 92% chemically modified nucleotides; at least 95% chemically modified nucleotides; at least 97% chemically modified nucleotides. In embodiments, a TDSC described herein, or one strand (e.g., the sense strand or the antisense strand) of the TDSC, comprises chemically modified nucleotides at between 0%-100% of each distinct nucleotide, e.g., 0%-100% chemically modified T nucleotides, 0%-100% chemically modified A nucleotides, 0%-100% chemically modified C nucleotides, and 0%-100% chemically modified G nucleotides for each construct. In embodiments, a TDSC described herein, or one strand (e.g., the sense strand or the antisense strand) of the TDSC, comprises chemically modified nucleotides at between 0-100%, 10%-100%, 20%-100%, 30%-100%, 40%-100%, 50%-100%, 60%-100%, 10%-50% of each distinct nucleotide, e.g., between 0-100%, 10%-100%, 20%-100%, 30%-100%, 40%-100%, 50%-100%, 60%-100%, 10%-50% of chemically modified T nucleotides; between 0-100%, 10%-100%, 20%-100%, 30%-100%, 40%-100%, 50%-100%, 60%-100%, 10%-50% of chemically modified A nucleotides; between 0-100%, 10%-100%, 20%-100%, 30%-100%, 40%-100%, 50%-100%, 60%-100%, 10%-50% of chemically modified C nucleotides; or between 0-100%, 10%-100%, 20%-100%, 30%-100%, 40%-100%, 50%-100%, 60%-100%, 10%-50% of chemically modified G nucleotides. For example, a TDSC could contain 100% chemically modified T nucleotides, 50% chemically modified A nucleotides, 0% chemically modified C nucleotides, and 25% chemically modified G nucleotides.

In embodiments, chemically modified nucleotides, e.g., modifications described herein, can be introduced in the TDSCs described herein throughout the entire sequence; within an element of a sequence, e.g., an element described herein; at a 5′- or 3′-end; and/or between the last 10, 8, 6, 5, 4, 3, or 2 nucleotides at the 5′- or 3′-end.

In some embodiments, a TDSC as described herein comprises chemically modified nucleotides on only one strand (e.g., as shown in FIG. 1A). In some embodiments, a TDSC as described herein comprises chemically modified nucleotides on the antisense strand. In some embodiments, a TDSC as described herein comprises chemically modified nucleotides on the sense strand.

In some embodiments, a TDSC as described herein comprises chemically modified nucleotides on both strands (e.g., as shown in FIGS. 1A and 2). In certain embodiments, both strands comprise chemical modifications at the same positions (e.g., chemically modified nucleotides on one strand are base-paired with chemically modified nucleotides on the opposite strand, and/or non-chemically modified nucleotides on one strand are base-paired with non-chemically modified nucleotides on the opposite strand). In embodiments, the entirety of both strands are composed of chemically modified nucleotides. In other embodiments, the two strands of a TDSC as described herein comprise different chemical modification patterns (e.g., one or more chemically modified nucleotides on one strand are base-paired with non-chemically modified nucleotides on the other strand). In embodiments, a TDSC as described herein comprises one or more double-stranded regions in which both strands are chemically modified, and/or one or more double-stranded regions in which neither strand is chemically modified. In embodiments, a TDSC as described herein comprises one or more double-stranded regions in which one strand is chemically modified and the other is not.

In embodiments, a TDSC as described herein comprises one or more DNA end forms (e.g., exonuclease-resistant DNA end forms, e.g., covalently closed DNA end forms or non-covalently closed DNA end forms, e.g., as described herein) that each comprise one or more chemically-modified nucleotides (e.g., on one or both strands of the DNA end form). In embodiments, a TDSC comprises a double-stranded region flanked by non-covalently closed exonuclease-resistant DNA end forms comprising chemically-modified nucleotides, e.g., as described herein (e.g., in FIG. 2).

In embodiments, a TDSC described herein has one or more chemical modification that disrupts the ability of a portion of the TDSC to form a double stranded structure, e.g., a TDSC described herein has one or more chemical modification on a nucleotide present in a region having intramolecular complementarity. In embodiments, a TDSC described herein has one or more chemical modification that disrupts base pairing of regions of intramolecular complementarity relative to the unmodified sequence of the TDSC. In some embodiments the chemically modified nucleotides used herein have a reduced propensity to base-pair with chemically modified nucleotides compared to the propensity of unmodified nucleotides to base pair with unmodified nucleotides. In some embodiments the chemically modified nucleotides used herein have an increased propensity to base-pair with unmodified nucleotides compared to modified nucleotides.

Other modifications are also contemplated. For example, ends of a linear DNA described herein can be chemically modified, e.g., to protect them from exonucleases. For example, one or more dideoxynucleotide residues can be added to the 3′ terminus of a linear molecule and/or self-complementary oligonucleotides are ligated to one or both ends. See, for example, Chang, et al. (1987) Proc. Nail. Acad. Sci. USA 84:4959-4963; Nehls, et at (1996) Science 272:886-889.

In some embodiments, a chemically modified TDSC described herein exhibits decreased recognition by DNA sensors in a host tissue or subject compared to an unmodified TDSC of the same sequence, e.g., at least 10%, 20%, 30%, 40%, 50%, 60%, 70%, 80%, 90%, 95% or more decreased recognition by DNA sensors in a host tissue or subject compared to an unmodified TDSC of the same sequence. In some embodiments, a chemically modified TDSC described herein exhibits decreased degradation by DNA nucleases compared to an unmodified TDSC of the same sequence, e.g., at least 10%, 20%, 30%, 40%, 50%, 60%, 70%, 80%, 90%, 95% or more decreased degradation by DNA nucleases in a host tissue or subject compared to an unmodified TDSC. In some embodiments, a chemically modified TDSC described herein shows decreased activation of the innate immune system in a target/host tissue or subject compared to an unmodified TDSC of the same sequence, e.g., at least 10%, 20%, 30%, 40%, 50%, 60%, 70%, 80%, 90%, 95% or more decreased activation of the innate immune system in a target/host tissue or subject compared to an unmodified TDSC of the same sequence.

In some embodiments, a TDSC comprising chemically modified nucleotides described herein exhibits any of the following properties in a target/host tissue or subject compared to dsDNA of the same sequence that does not comprise chemically modified nucleotides (unmodified dsDNA): increased integration of exogenous construct in genome of target cell; increased retention in a target cell through replication; reduced secondary or tertiary structure formation; reduced interaction with innate immune sensors; reduced interaction with nucleases; enhanced stability; enhanced longevity; reduced toxicity; enhanced delivery; increased expression; increased transport across membranes; increased binding to DNA binding moieties such as nuclear DNA binding proteins, transcription factors, chaperones, DNA polymerases. In embodiments, any of the above listed properties is modulated at least 10%, 20%, 30%, 40%, 50%, 60%, 70%, 80%, 90%, 95% or more in a target/host tissue or subject compared to an unmodified dsDNA of the same sequence.

### Structure of DNA Constructs

In some embodiments, the TDSC or nucleic acid comprising dsDNA disclosed herein is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 500 nucleotides, at least about 1000 nucleotides, at least about 2000 nucleotides, at least about 3000 nucleotides, at least about 4000 nucleotides, at least about 5000 nucleotides, at least about 6000 nucleotides, at least about 7000 nucleotides, at least about 8000 nucleotides, at least about 9000 nucleotides, at least about 10,000 nucleotides, at least about 20,000 nucleotides, at least about 30,000 nucleotides, at least about 40,000 nucleotides, or at least about 50,000 nucleotides in length. In some embodiments, the TDSC or nucleic acid comprising dsDNA disclosed herein is between 20-30, 30-40, 40-50, 50-75, 75-100, 100-200, 200-300, 300-500, 500-1000, 1000-2000, 2000-3000, 3000-4000, 4000-5000, 5000-6000, 6000-7000, 7000-8000, 8000-9000, 9000-10,000, 10,000-20,000, 20,000-30,000, 30,000-40,000, or 40,000-50,000 nucleotides in length. In some embodiments, the size of a TDSC disclosed herein is a length sufficient to encode useful polypeptides or RNAs.

In some embodiments, a TDSC or nucleic acid comprising dsDNA comprises an exonuclease-resistant DNA end form (e.g., as described herein). In some embodiments, the DNA end form is at least 2, 3, 4, 5, 10, 15, 20, 25, 30, 40, 50, 60, 70, 80, 90, or 100 nucleotides in length. In some embodiments, the DNA end form is less than 10, 15, 20, 25, 30, 40, 50, 60, 70, 80, 90, or 100 nucleotides in length. In some embodiments, the DNA end form is 2-5, 5-10, 10-15, 15-20, 20-25, 25-30, 30-35, 35-40, 40-45, 45-50, 50-55, 55-60, 60-70, 70-80, 80-90, or 90-100 nucleotides in length.

In some embodiments, a TDSC or nucleic acid comprising dsDNA comprises double stranded region encoding an effector (e.g., a polypeptide or RNA, e.g., as described herein), e.g., positioned between two exonuclease-resistant DNA end forms. In some embodiments, the double stranded region is at least 10, 15, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, or 50,000 nucleotides in length. In some embodiments, the double stranded region form is less than 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, or 50,000 nucleotides in length. In some embodiments, the double stranded region is 10-15, 15-20, 20-25, 25-30, 30-35, 35-40, 40-45, 45-50, 50-55, 55-60, 60-70, 70-80, 80-90, 90-100, 100-200, 200-300, 300-400, 400-500, 500-600, 600-700, 700-800, 800-900, 900-1000, 1000-2000, 2000-3000, 3000-4000, 4000-5000, 5000-6000, 6000-7000, 7000-8000, 8000-9000, 9000-10,000, 10,000-20,000, 20,000-30,000, 30,000-40,000, or 40,000 to 50,000 nucleotides in length.

A TDSC described herein may have less than a threshold level of single stranded structures. In one embodiment, the TDSC does not comprise more than 20, 18, 16, 14, 12, 10, 8, 7, 5, 4, 3, 2, or 1 single stranded region longer than 100, 80, 70, 60, 50, 40, 30, 20 or 10 bases, e.g., does not comprise single stranded regions longer than 100, 80, 70, 60, 50, 40, 30, 20 or 10 bases. In one embodiment, double stranded regions formed by a TDSC described herein is determined as described by Xayaphoummine et al. 2005. Kinefold web server for RNA/DNA folding path and structure prediction including pseudoknots and knots. Nucleic Acids Research, Volume 33: W605-610. In one embodiment, the Kinefold website (http://kinefold.curie.fr/cgi-bin/form.pl) is used to predict double stranded regions of a construct described herein, using the following parameters:
Sequence to fold: enter and select “DNA sequence”
Stochastic Simulation: Co-transcriptional fold, 3 milliseconds
Simulated molecular time: default
Pseudoknots: not allowed
Entanglements: non crossing
Random seed: 11453

### Production

In some embodiments, a TDSC or nucleic acid comprising dsDNA as described herein is produced from a plasmid assembled to contain the desired elements described herein. The plasmid template can be assembled, for example, using Golden Gate cloning for assembly of multiple DNA fragments in a defined linear order in a recipient vector using a one-pot assembly procedure. Golden Gate cloning is described in Marillonnet & Grützner, 2020, Synthetic DNA assembly using golden gate cloning and the hierarchical modular cloning pipeline, Current Protocols in Molecular Biology, 130: e115. In some embodiments, a plasmid template is linearized, for example, by digestion with a nuclease (e.g., a restriction endonuclease) or by PCR amplification of a linear nucleic acid sequence from the plasmid template (e.g., as described in Example 2). In certain embodiments, linearization of the plasmid template produces a proto-TDSC as described herein (e.g., a linear nucleic acid comprising dsDNA that does not comprise an exonuclease-resistant DNA end form at one or both ends).

In some embodiments, a TDSC or proto-TDSC comprising chemical modifications on one strand is produced by amplification of one strand (e.g., from a plasmid template) using a dNTP mixture comprising one or more chemically modified nucleotides and a primer that can amplify one strand of the TDSC or proto-TDSC sequence (e.g., as described in Example 3). In certain embodiments, the opposite strand (e.g., an unmodified strand or a differently chemically modified strand, e.g., as described herein, for example, in FIGS. 1A-2) is produced in a separate amplification reaction, e.g., using a dNTP mixture comprising unmodified nucleotides or a different set of chemically modified nucleotides, and a primer that can amplify the opposite strand of the TDSC or proto-TDSC sequence (e.g., as described in Example 3).

In some embodiments, a TDSC or proto-TDSC comprising the same chemical modification(s) on both strand is produced by amplification of the TDSC or proto-TDSC strands (e.g., from a plasmid template) using a dNTP mixture comprising one or more chemically modified nucleotides and primers that can amplify both strand of the TDSC or proto-TDSC sequence (e.g., as described in Example 4).

In some embodiments, an exonuclease-resistant DNA end form (e.g., as described herein) is introduced (e.g., attached) to one or both ends of a proto-TDSC. In certain embodiments, the DNA end form is attached to an end of the proto-TDSC by ligation (e.g., as described in Example 5 or 6). In embodiments, attachment (e.g., ligation) of the DNA end form (e.g., a covalently closed DNA end form) to the proto-TDSC produces the final TDSC. In certain embodiments, exonuclease resistance of the attached DNA end form is confirmed, for example, by incubating the TDSC in the presence of an exonuclease (e.g., Exonuclease III, USER enzyme, and/or Mung Bean Nuclease), e.g., as described in Examples 10 and 11. In embodiments, exonuclease resistance of the attached DNA end form is confirmed, for example, by incubating the TDSC in the presence of Exonuclease III. In embodiments, the DNA end form comprises a blunt end, sticky end, or Y-adaptor (e.g., as described herein), and the exonuclease resistance of the attached DNA end form is confirmed by incubating the TDSC in the presence of Exonuclease III and (e.g., subsequently, prior to, or concurrently) Mung Bean nuclease and/or USER enzyme.

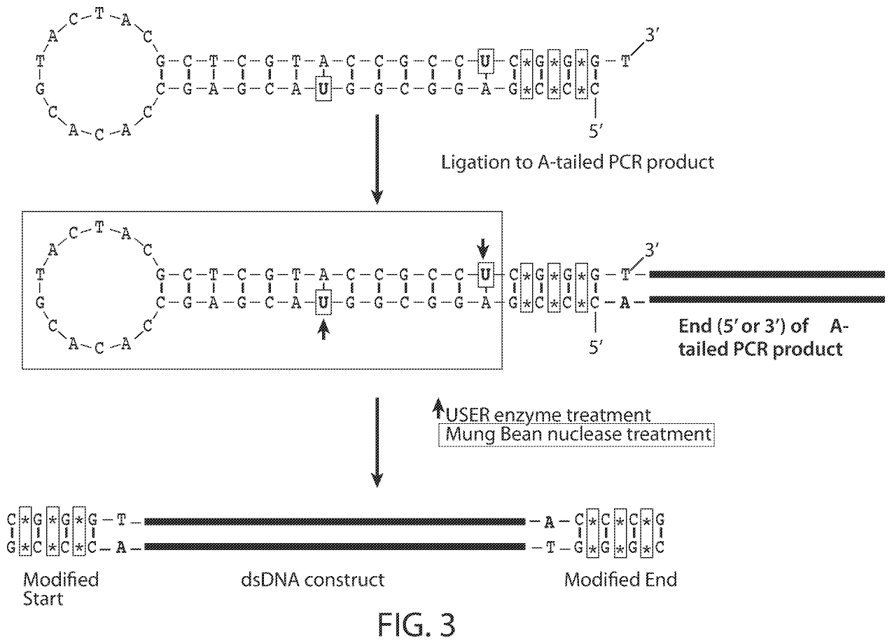

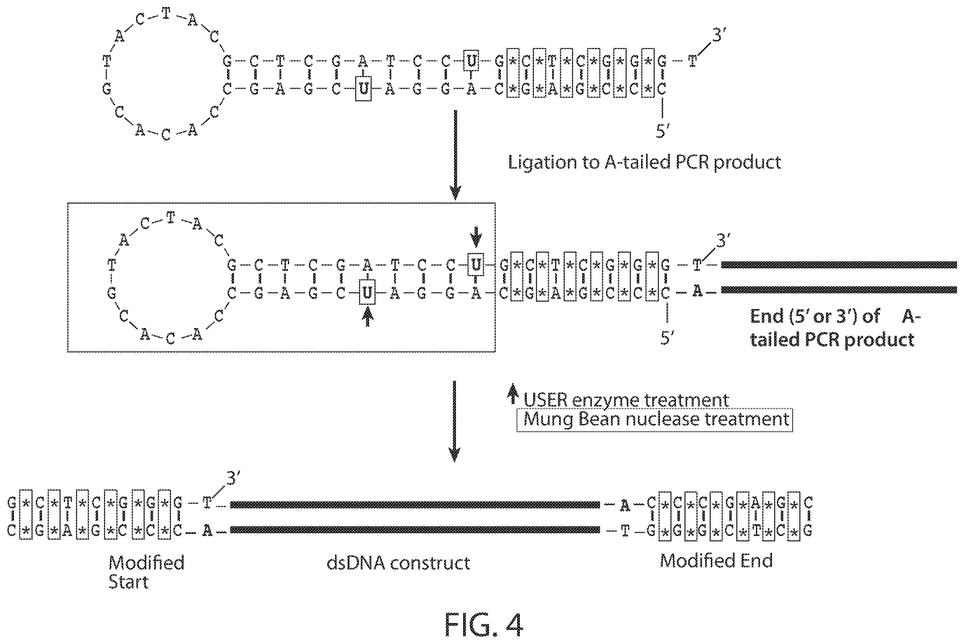

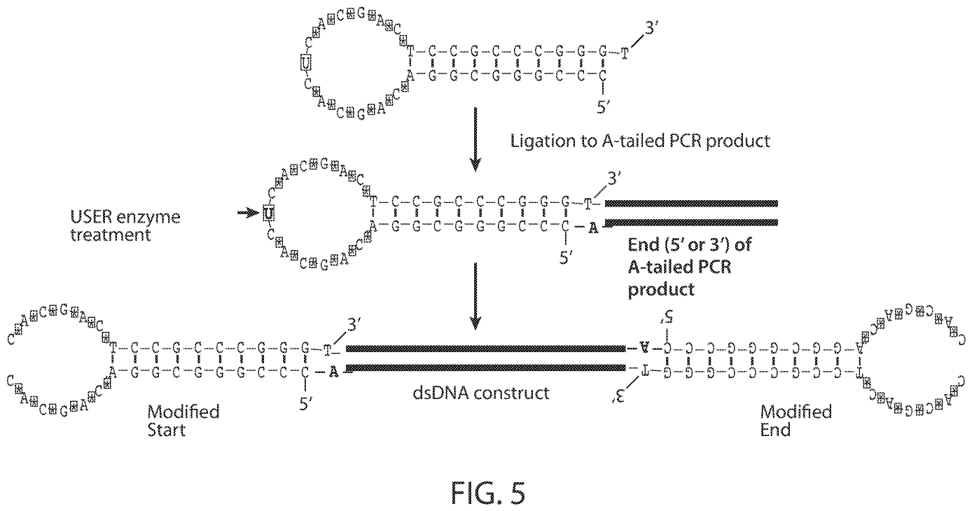

In certain embodiments, the DNA end form is attached to the end of the proto-TDSC in a nascent form (e.g., a non-covalently closed DNA end form may be attached to the proto-TDSC as a hairpin, e.g., as described in Example 5 and FIGS. 3-4). In a subsequent step, the nascent form of the DNA end form may be further modified (e.g., cleaved) to produce the final DNA end form. For example, a non-covalently closed DNA end form may be produced by cleavage of a nascent form, e.g., by a nuclease. In embodiments, the nascent form comprises one or more uracil nucleotides. In embodiments, the nascent form is cleaved at the one or more uracil nucleotides using a USER enzyme. In some embodiments, a nascent form comprising an overhang or sticky end (e.g., a nascent form produced by USER enzyme cleavage as shown in FIGS. 3-4) can be converted to a blunt end by digestion with a single strand-specific nuclease, e.g., a Mung Bean nuclease (e.g., as described in Example 5). In some embodiments, a nascent form comprising a hairpin comprising a cleavable moiety (e.g., a uracil nucleotide) in its single-stranded loop region is converted to a Y-adaptor by cleavage of the cleavable moiety (e.g., by a USER enzyme), e.g., as described in Example 7. T

The TDSC may be enriched or purified from impurities or byproducts selected from the group consisting of: endotoxin, mononucleotides, chemically modified mononucleotides, single stranded DNA, circular DNA, proteins (e.g., enzymes, e.g., ligases, restriction enzymes), DNA fragments or truncations. In some embodiments, the purified TDSC is substantially free of process byproducts and impurities, e.g., process byproducts or impurities described herein.

In some embodiments, a TDSC is formulated with a lipid based carrier, e.g., a lipid nanoparticle (LNP), e.g., as described in Example 8.

The TDSC may be sequenced to confirm the desired, designed sequence. In embodiments, other structural analysis of the TDSC (e.g., restriction enzyme analysis) may be performed to confirm or verify its sequence.

### Pharmaceutical Compositions

The present disclosure includes TDSC or nucleic acid comprising dsDNA and related compositions in combination with one or more pharmaceutically acceptable excipients and/or carriers.

Pharmaceutical compositions may optionally comprise one or more additional active substances, e.g., therapeutically and/or prophylactically active substances. Pharmaceutical compositions of the present invention are generally sterile and/or pyrogen-free.

A TDSC described herein may be formulated without a carrier, e.g., the TDSC described herein may be administered to a host cell, tissue or subject “naked”. A naked formulation may include pharmaceutical excipients or diluents but lacks a carrier.

Pharmaceutically acceptable excipients or diluents may comprise an inactive substance that serves as a vehicle or medium for the compositions described herein, such as any one of the inactive ingredients approved by the United States Food and Drug Administration (FDA) and listed in the Inactive Ingredient Database, which is incorporated by reference herein. Non-limiting examples of pharmaceutically acceptable excipients or diluents include solvents, aqueous solvents, non-aqueous solvents, tonicity agents, dispersion media, cryoprotectants, diluents, suspension aids, surface active agents, isotonic agents, thickening agents, emulsifying agents, preservatives, hyaluronidases, dispersing agents, preservatives, lubricants, granulating agents, disintegrating agents, binding agents, antioxidants, buffering agents (e.g., phosphate buffered saline (PBS)), lubricating agents, oils, and mixtures thereof.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

### Carriers

A TDSC or nucleic acid comprising dsDNA described herein may also be formulated, or included, with a carrier. General considerations of carriers and delivery of pharmaceutical agents may be found, for example, in Delivery Technologies for Biopharmaceuticals: Peptides, Proteins, Nucleic Acids and Vaccines (Lene Jorgensen and Hanne Morck Nielson, Eds.) Wiley; 1 st edition (Dec. 21, 2009); and Vargason et al. 2021. Nat Biomed Eng 5, 951-967.

Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the TDSC, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the TDSC), peptides (e.g., cell-penetrating peptides), materials (e.g., graphene oxide), single pure lipids (e.g., cholesterol), DNA origami (e.g., DNA tetrahedron).

In one embodiment, the TDSC compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for an agent (e.g., a TDSC) described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111 (28): 10131-10136; U.S. Pat. No. 9,644,180; Huang et al. 2017. Nature Communications 8:423; Shi et al. 2014. Proc Natl Acad Sci USA. 111 (28): 10131-10136.

Fusosome compositions, e.g., as described in WO2018208728, can also be used as carriers to deliver the TDSCs described herein.

### Lipid Nanoparticles:

Lipid nanoparticles (LNPs) are carriers made of ionizable lipids. LNPs are taken up by cells via endocytosis, and their properties allow endosomal escape, which allows release of the cargo into the cytoplasm of a target cell. In addition to ionizable lipids, LNPs may contain a helper lipid to promote cell binding, cholesterol to fill the gaps between the lipids, and/or a polyethylene glycol (PEG) to reduce opsonization by serum proteins and reticuloendothelial clearance. Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference—e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-1-0-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid described herein includes,

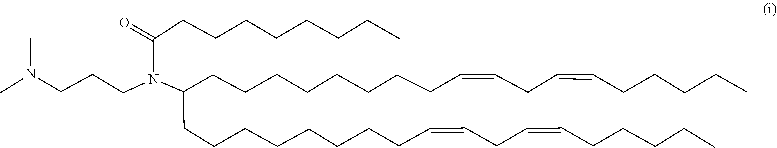 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a DNA composition described herein to the liver and/or hepatocyte cells.

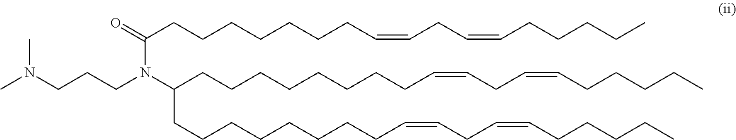 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a DNA composition described herein to the liver and/or hepatocyte cells.

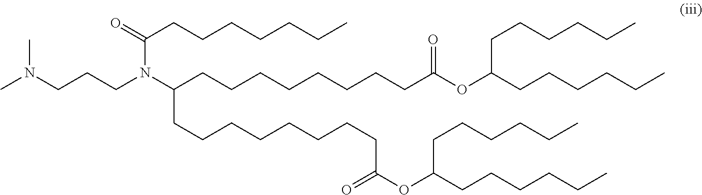 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a DNA composition described herein to the liver and/or hepatocyte cells.

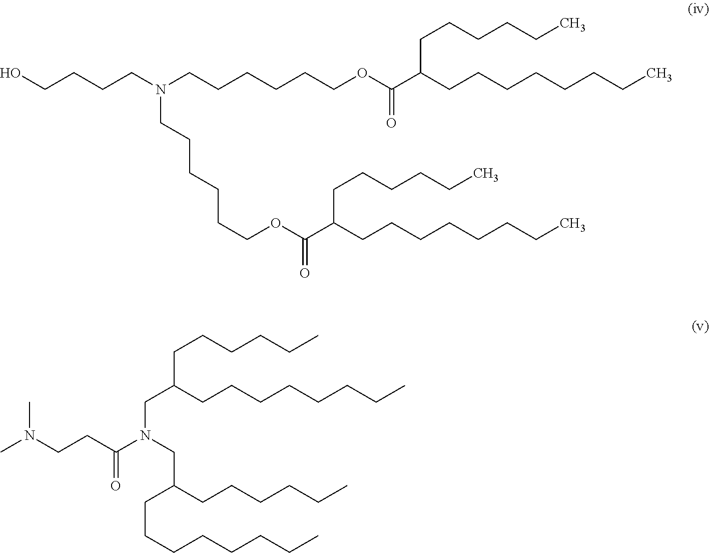

In some embodiments an LNP comprising Formula (v) is used to deliver a DNA composition described herein to the liver and/or hepatocyte cells.

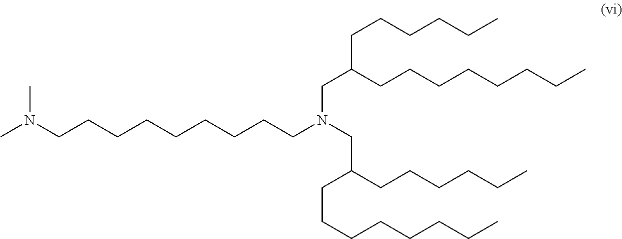 [SMILES: CCCCCCCCC(CCCCCC)CN(CCCCCCCCCN(C)C)CC(CCCCCC)CCCCCCCC]

In some embodiments an LNP comprising Formula (vi) is used to deliver a DNA composition described herein to the liver and/or hepatocyte cells.

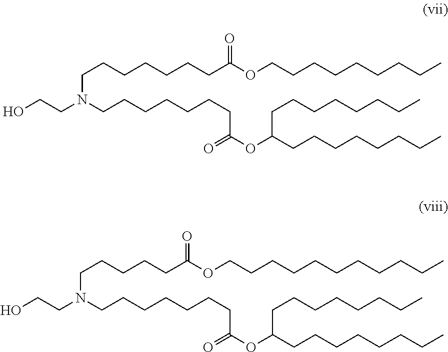 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula vii or (viii) is used to deliver a DNA composition described herein to the liver and/or hepatocyte cells.

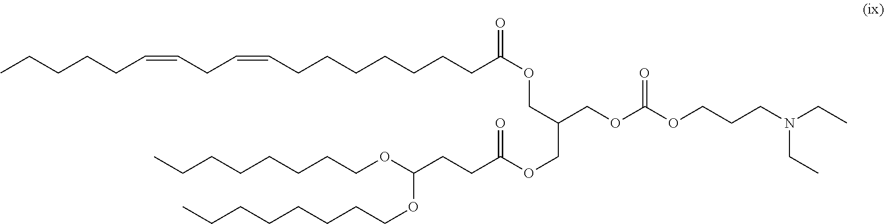 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a DNA composition described herein to the liver and/or hepatocyte cells.

In some embodiments an LNP comprising Formula (x) is used to deliver a DNA composition described herein to the liver and/or hepatocyte cells:

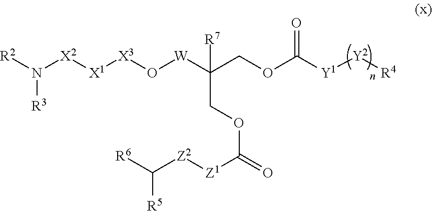

wherein
X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4—, 5., or 6-membered ring, or X′ is NR′, R′ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

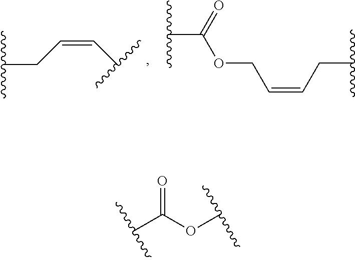 [SMILES: */C=C\C* ; *C/C=C\COC(*)=O ; *OC(*)=O]

(in either orientation), (in either orientation), (in either orientation),
n is 0 to 3, R⁴ is Ci-15 alkyl, Z¹ is Ci-6 alkylene or a direct bond,
Z² is

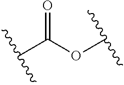 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

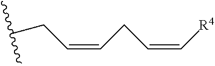

R⁴ is linear C5 alkyl, Z¹ is C2 alkylene, Z² is absent, W is methylene, and R⁷ is H, then R⁵ and R⁶ are not Cx alkoxy.

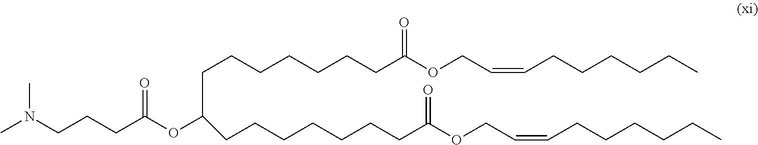 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a DNA composition described herein to the liver and/or hepatocyte cells.

In some embodiments an LNP comprising Formula (xii) is used to deliver a DNA composition described herein to the liver and/or hepatocyte cells.

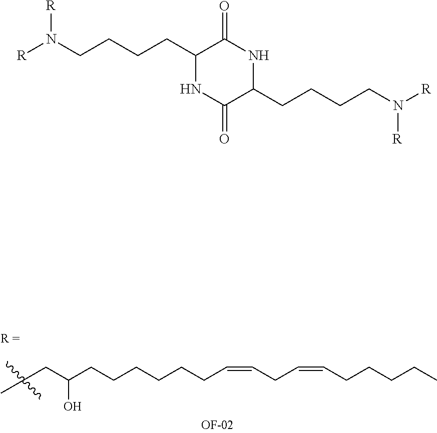

where R=

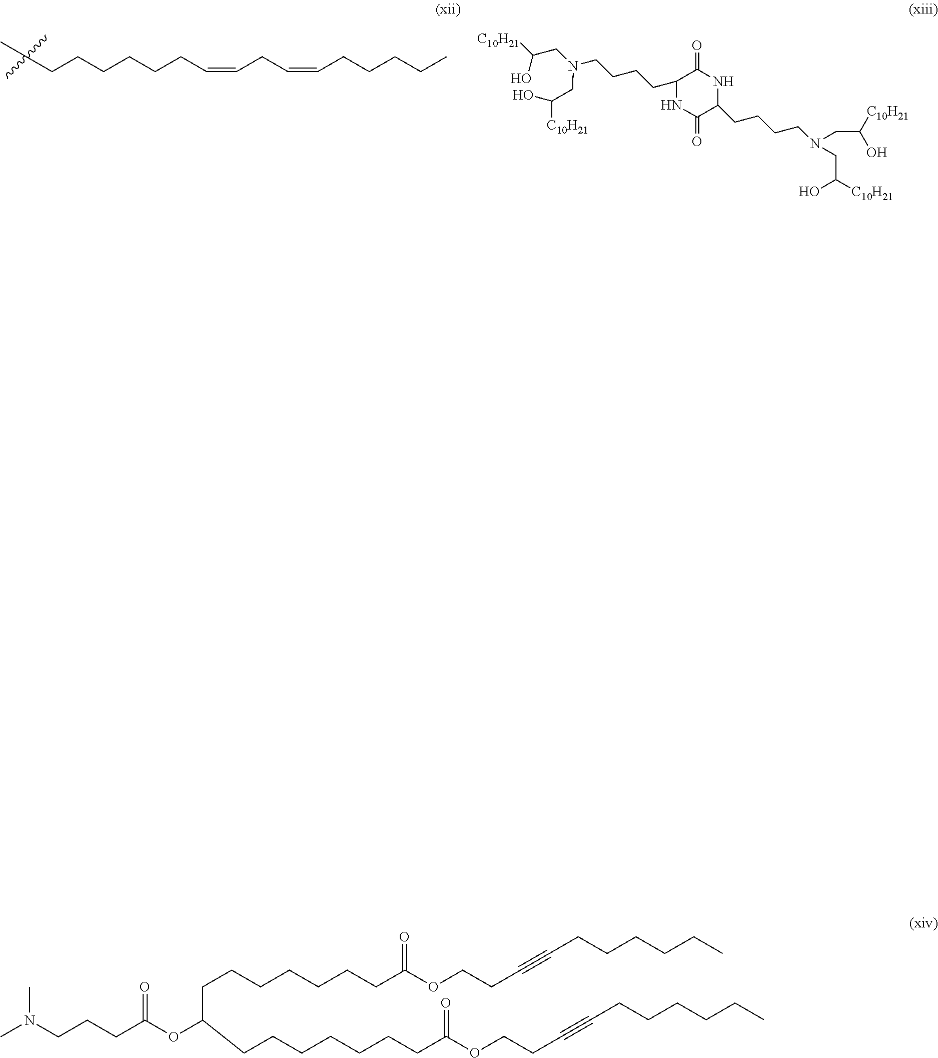

In some embodiments an LNP comprises a compound of Formula (xiii) and a compound of Formula (xiv).

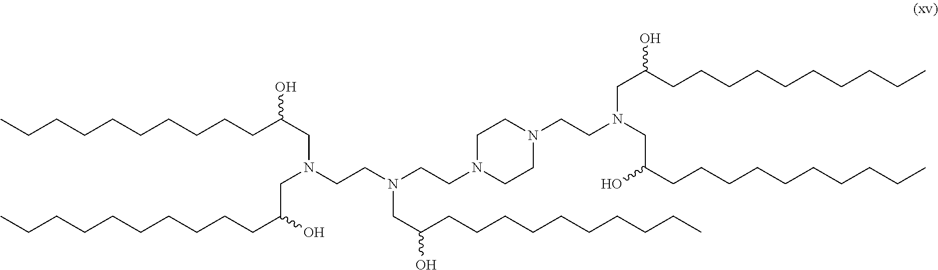 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a DNA composition described herein to the liver and/or hepatocyte cells.

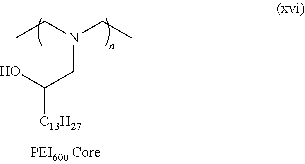

In some embodiments an LNP comprising a formulation of Formula (xvi) is used to deliver a DNA composition described herein to the lung endothelial cells.

In some embodiments an LNP comprising a formulation of Formula (xvii), (xviii), or (xix) is used to deliver a DNA composition described herein to the lung endothelial cells.

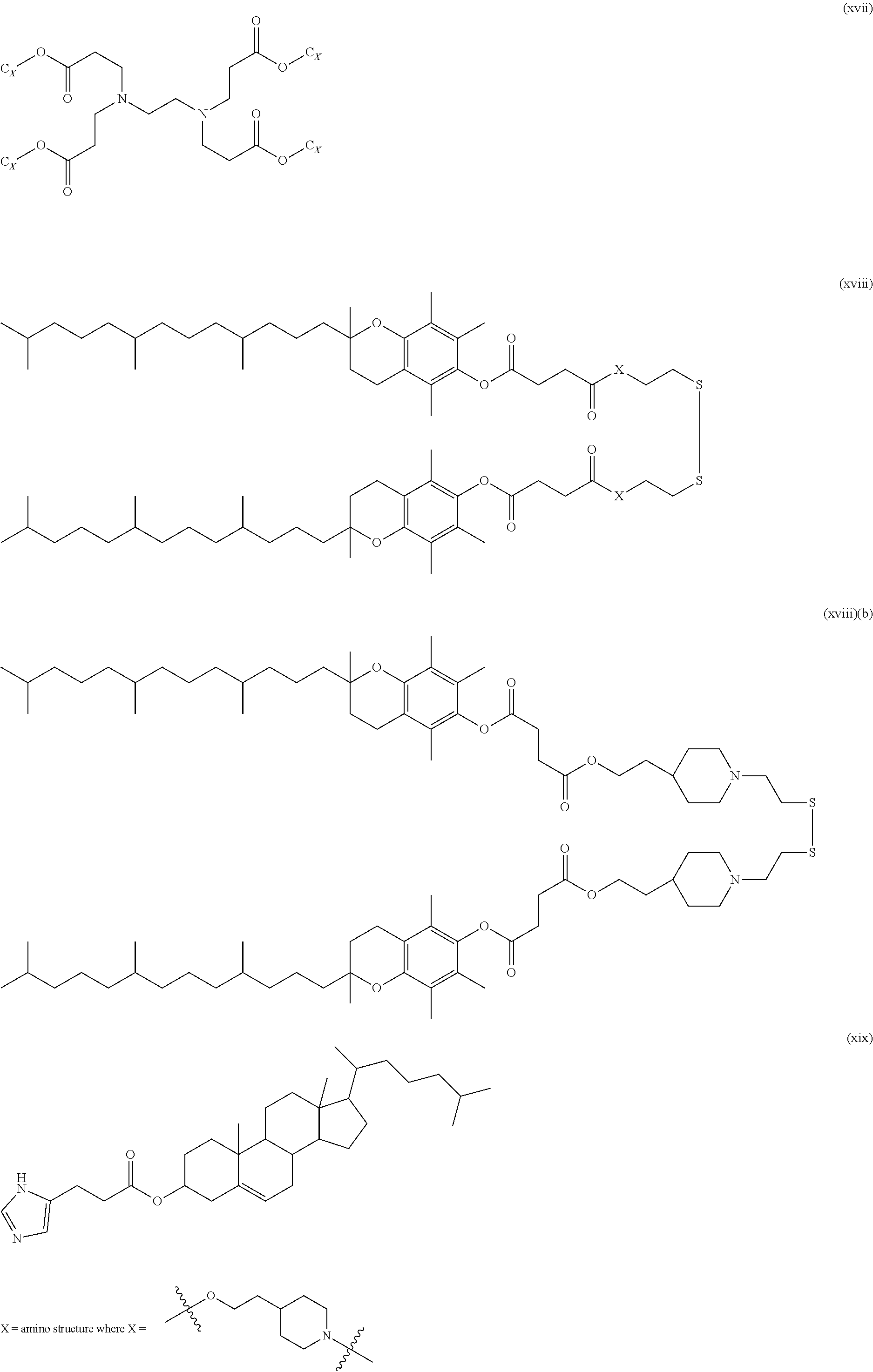

In some embodiments, a lipid compound used to form lipid nanoparticles for the delivery of compositions described herein, e.g., nucleic acid described herein is made by one of the following reactions:

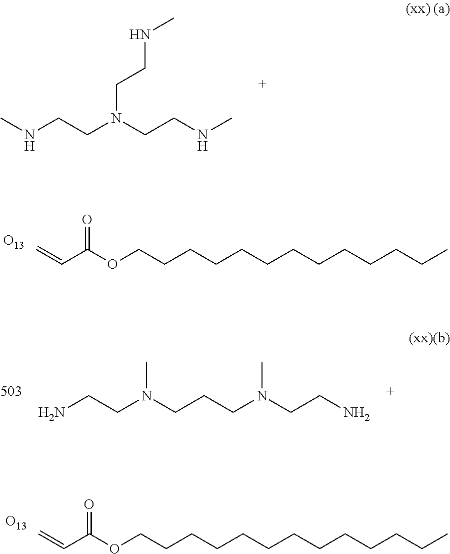

In some embodiments, a composition described herein (e.g., a nucleic acid or a protein) is provided in an LNP that comprises an ionizable lipid. In some embodiments, the ionizable lipid is heptadecan-9-yl 8-((2-hydroxyethyl) (6-oxo-6-(undecyloxy) hexyl)amino) octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy) butanoyl)oxy)-2-((((3-(diethylamino) propoxy) carbonyl)oxy)methyl) propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy) heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl) (2-hydroxydodecyl)amino)ethyl) piperazin-1-yl)ethyl) azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta [a]phenanthren-3-yl 3-(1H-imidazol-4-yl) propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid described herein, encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of molecule TDSC.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 lZ)-heptatriaconta-6,9,28,3 l-tetraen-19-yl-4-(dimethylamino)butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-l3, l6-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2:-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy) propyl-1-O-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In some embodiments, the PEG-lipid comprises PEG-DMG, 1,2-dimyristoyl-sn-glycero-3-phosphoethanolamine-N-[methoxy (polyethylene glycol)-2000]. In some embodiments, the PEG-lipid comprises a structure selected from:

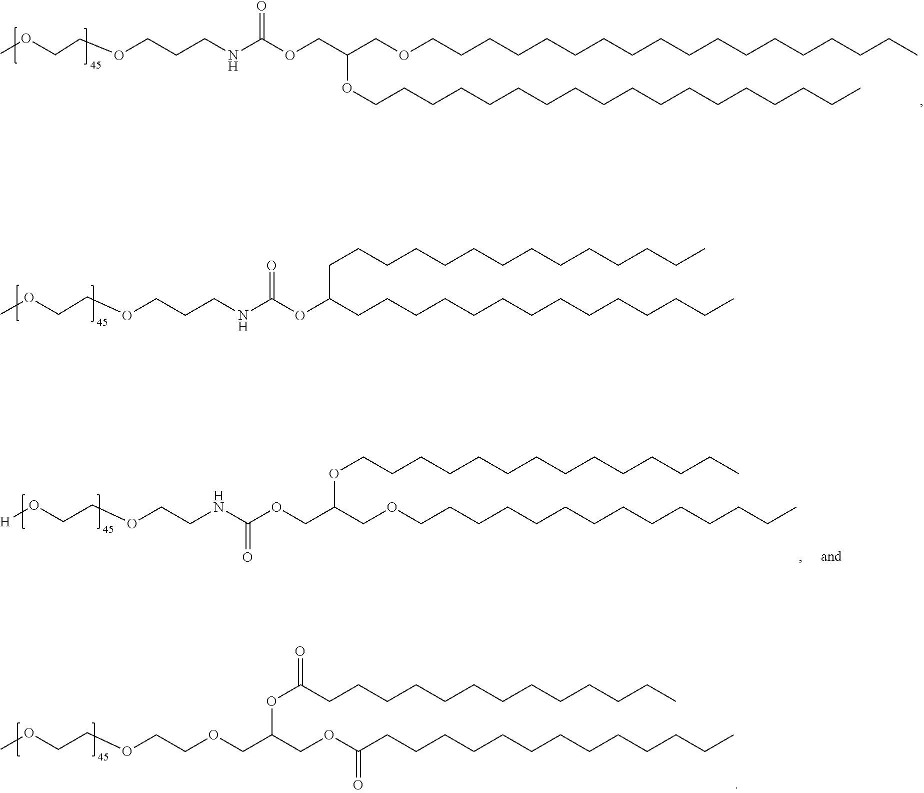

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18 (7): 1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15 (4): 313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino) propoxy) carbonyl)oxy)methyl) propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid in a solution. Fluorescence may be used to measure the amount of free protein and/or nucleic acid in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety. See also: Hou et al. Lipid nanoparticles for mRNA delivery. Nat Rev Mater (2021). https://doi.org/10.1038/s41578-021-00358-0.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of a DNA described herein with an LNP may include about 0.1, 0.25, 0.3, 0.5, 1, 2, 3, 4, 5, 6, 8, 10, or 100 mg/kg (DNA).

The following embodiments are contemplated:
A. A lipid nanoparticle (LNP) comprising a TDSC construct, sequence or composition described herein.
B. The LNP of embodiment A, comprising a cationic lipid.
C. The LNP of embodiment B, wherein the cationic lipid has a structure according to:

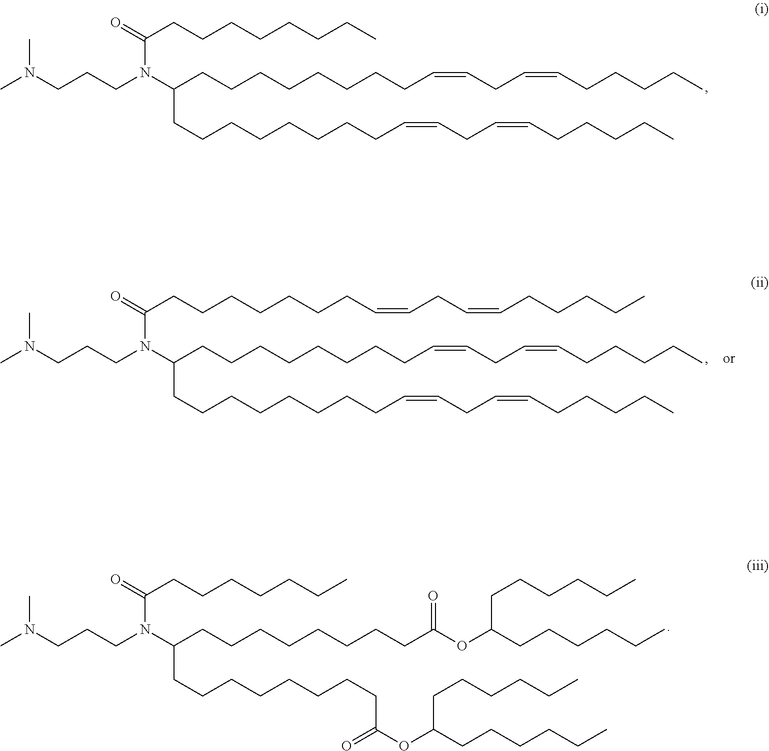

D. The LNP of any of embodiments A-C, further comprising one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

In embodiments, an LNP preparation comprising a TDSC described herein can be targeted to the desired cell type by surface decoration with targeting effectors. Such targeting effectors include, e.g., cell specific receptor ligands that bind a target cell; antibodies or other binders against a target cell; centryins; cell penetrating peptides; peptides that enable endosomal escape (e.g., GALA, KALA). See, e.g., Tables 1 and 2 of Tai & Gao. 2017. Adv Drug Deliv Rev. 110-111:157-168, for a review.

In embodiments, an LNP preparation comprising a TDSC described herein can be co-administered with an adjuvant, e.g., co-delivered in the same preparation with an adjuvant.

### Route of Administration

A TDSC or nucleic acid comprising dsDNA described herein is introduced into a cell, tissue or subject by any suitable route.

Administration to a target cell or tissue (e.g., ex vivo) may be by methods known in the art such as transfection, e.g., transient or stable transfection using reagents (e.g., liposomal, calcium phosphate) or physical means (e.g., electroporation, gene gun, microinjection, microfluidic fluid shear, cell squeezing). Other methods are described, e.g., in Rad et al. 2021. Adv. Mater. 33:2005363, which is incorporated herein by reference.

Administration to a subject, e.g., a mammal, e.g., a human subject, may be by parenteral (e.g., intravenous, intramuscular, intraperitoneal, subcutaneous, or intracranial) route; by topical administration, transdermal administration or transcutaneous administration. Other suitable routes include oral, rectal, transmucosal, intranasal, inhalation (e.g., via an aerosol), buccal (e.g., sublingual), vaginal, intrathecal, intraocular, transdermal, intraendothelial, in utero (or in ovo), intrapleural, intracerebral, intraarticular, topical, intralymphatic. Also included is direct tissue or organ injection (e.g., to liver, eye, skeletal muscle, cardiac muscle, diaphragm, muscle or brain).

### Applications

The TDSC or nucleic acid comprising dsDNA described herein can be used in therapeutic or health applications for a subject, e.g., a human or non-human animal. Although the descriptions of pharmaceutical compositions provided herein are principally directed to pharmaceutical compositions which are suitable for administration to humans, it will be understood by the skilled artisan that such compositions are generally suitable for administration to any other animal. The subject can be any animal, e.g., a mammal, e.g., a human or non-human mammal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk.

In some embodiments, a DNA described herein is provided at a dose of about 0.1-100 mg/kg of the DNA.

In some embodiments, a TDSC described herein imparts a biological effect of the effector, e.g., expression of a therapeutic polypeptide, on a host cell, tissue or subject over a time period of at least 2, 3, 4, 5, 6 days or a week; at least 8, 9, 10, 12, 14 days or two weeks; at least 16, 18, 20 days or 3 weeks; at least 22, 24, 25, 27, 28 days or a month; at least 2 months, 3 months, 4 months, 5 months, 6 months or more; between one week and 6 months, between 1 month to 6 months, between 3 months to 6 months.

In some embodiments, a TDSC described herein imparts a biological effect of the effector, e.g., expression of a therapeutic polypeptide, on a host cell, tissue or subject over a time period of at least 1 cell divisions of the host cell.

In embodiments, a TDSC described herein can be used to deliver an effector, e.g., an effector described herein, to a cell, tissue or subject.

In embodiments, a TDSC described herein can be used to modulate (e.g., increase or decrease) a biological parameter in a cell, tissue or subject. The biological parameter may be an increase or decrease in gene expression of a subject gene in a target cell, tissue or subject. In embodiments, a TDSC described herein can be used to treat a cell, tissue or subject in need thereof by administering a TDSC described herein to such cell, tissue or subject.

In embodiments, the TDSC delivers an effector to a cell chosen from a lymphocyte (e.g., a T cell or a monocyte), a cancer cell (e.g., an osteosarcoma cell), a HEK293 cell, a hepatocyte, or an epidermal cell (e.g., a keratinocyte).

### EXAMPLES

### Example 1: Design and Assembly of a Plasmid Template for a Linear TDSC

This example describes how to create a plasmid template for a chemically modified, linear TDSC construct. In this example, a construct template is designed with the following specific sequence components:

Promoter Ef1a:
(SEQ ID NO: 37)
5′ggctccggtgcccgtcagtgggcagagcgcacatcgcccacag
tccccgagaagttggggggaggggtcggcaattgaaccggtgcct
agagaaggtggcgcggggtaaactgggaaagtgatgtcgtgtact
ggctccgcctttttcccgagggtgggggagaaccgtatataagtg
cagtagtcgccgtgaacgttctttttcgcaacgggtttgccgcca
gaacacaggtaagtgccgtgtgtggttcccgcgggcctggcctct
ttacgggttatggcccttgcgtgccttgaattacttccacctggc
tgcagtacgtgattcttgatcccgagcttcgggttggaagtgggg
ggagagttcgaggccttgcgcttaaggagccccttcgcctcgtgc
ttgagttgaggcctggcctgggcgctggggccgccgcgtgcgaat
ctggtggcaccttcgcgcctgtctcgctgctttcgataagtctct
agccatttaaaatttttgatgacctgctgcgacgctttttttctg
gcaagatagtcttgtaaatgcgggccaagatctgcacactggtat
ttcggtttttggggccgcgggcggcgacggggcccgtgcgtccca
gcgcacatgttcggcgaggcggggcctgcgagcgcggccaccgag
aatcggacgggggtagtctcaagctggccggcctgctctggtgcc
tggcctcgcgccgccgtgtatcgccccgccctgggcggcaaggct
ggcccggtcggcaccagttgcgtgagcggaaagatggccgcttcc
cggccctgctgcagggagctcaaaatggaggacgcggcgctcggg
agagcggggggtgagtcacccacacaaaggaaaagggcctttccg
tcctcagccgtcgcttcatgtgactccacggagtaccgggcgccg
tccaggcacctcgattagttctcgagcttttggagtacgtcgtct
ttaggttggggggaggggttttatgcgatggagtttccccacact
gagtgggtggagactgaagttaggccagcttggcacttgatgtaa
ttctccttggaatttgccctttttgagtttggatcttggttcatt
ctcaagcctcagacagtggttcaaagtttttttcttccatttcag
gtgtcgtga-3′
Effector sequence encoding a model/marker
protein (mCherry):
(SEQ ID NO: 38)
5′atggtgagcaagggcgaggaggataacatggccatcatcaagg
agttcatgcgcttcaaggtgcacatggagggctccgtgaacggcc
acgagttcgagatcgagggcgagggcgagggccgcccctacgagg
gcacccagaccgccaagctgaaggtgaccaagggtggccccctgc
ccttcgcctgggacatcctgtcccctcagttcatgtacggctcca
aggcctacgtgaagcaccccgccgacatccccgactacttgaagc
tgtccttccccgagggcttcaagtgggagcgcgtgatgaacttcg
aggacggcggcgtggtgaccgtgacccaggactcctccctgcagg
acggcgagttcatctacaaggtgaagctgcgcggcaccaacttcc
cctccgacggccccgtaatgcagaagaagaccatgggctgggagg
cctcctccgagcggatgtaccccgaggacggcgccctgaagggcg
agatcaagcagaggctgaagctgaaggacggcggccactacgacg
ctgaggtcaagaccacctacaaggccaagaagcccgtgcagctgc
ccggcgcctacaacgtcaacatcaagttggacatcacctcccaca
acgaggactacaccatcgtggaacagtacgaacgcgccgagggcc
gccactccaccggcggcatggacgagctgtacaagtaa-3′

Optionally, the construct may also include one or both of an NTS or a maintenance sequence, for example:

NTS: SV40 enhancer:
(SEQ ID NO: 1)
5′-cccaagaagaagaggaaagtc-3′
Maintenance sequence: human interferon-α MAR
(SEQ ID NO: 121)
5′tataattcactggaatttttttgtgtgtatggtatgacatatg
ggttcccttttattttttacatataaatatatttccctgtttttc
taaaaaagaaaaagatcatcattttcccattgtaaaatgccatat
ttttttcataggtcacttacata3′
PolyA site:
(SEQ ID NO: 122)
5′ctgtgccttctagttgccagccatctgttgtttgcccctcccc
cgtgccttccttgaccctggaaggtgccactcccactgtcctttc
ctaataaaatgaggaaattgcatcgcattgtctgagtaggtgtca
ttctattctggggggtggggggggcaggacagcaagggggaggat
tgggaagacaatagcaggcatgctggggatgcggtgggctctatg
g3′

A plasmid template is designed with these elements using standard DNA design manipulation software. Assembly is performed via Golden Gate Assembly following published protocols and commercially available kits (Marillonnet & Grützner. 2020. Synthetic DNA assembly using golden gate cloning and the hierarchical modular cloning pipeline. Current Protocols in Molecular Biology. 130: e115; Golden Gate Assembly Protocol for Using NEB Golden Gate Assembly Mix (E1600) (New England Biolabs). Golden Gate assembly of the designed construct is performed using a series of primers 120 bp long with the first 30 bp matching the relevant adjacent fragment, the next 60 bp encoding for the new sequence, and the final 30 bp annealing to the target sequence. Fragments are assembled into the final construct design (NEB Golden Gate Assembly Kit) and sequences are confirmed by Sanger Sequencing (Sigma Aldrich) according to manufacturer protocols.

### Example 2: Conversion of Plasmid DNA to an Unmodified TDSC

This example describes the creation of a TDSC comprising linear dsDNA, without chemically modified nucleotides. In this example, linear dsDNA is made from the plasmid in Example 1 using PrimeSTAR® Max DNA Polymerase according to manufacturer protocols (Takara, R045Q). The dsDNA is purified using NucleoSpin® Gel and PCR Clean-Up according to manufacturer protocols (Takara, 740609) and the concentration of the dsDNA is determined by Qubit™ 1×dsDNA Broad Range according to manufacturer protocols (Thermo Fisher, Q33265).

### Example 3: Conversion of Plasmid DNA to a TDSC that Only has the Sense or Antisense Strand Chemically Modified

This example describes generation of a TDSC comprising linear dsDNA that only has chemical modifications to the sense or antisense strand. The plasmid construct in Example 1 is converted into linear ssDNA following the methods in Minev et al., 2019, Rapid in vitro production of single stranded DNA, Nucleic Acids Research, Volume 47, Issue 22:11956-11962, which is incorporated herein by reference. Briefly, 5-methylcytosine (5mC), the chemically modified nucleotide, is mixed with standard adenine (dATP), guanine (dGTP), and thymine (dTTP) to create the dNTP mix for PCR to create the chemically modified strand. PCR with a forward primer bearing a methanol responsive polymer generates a tagged amplicon that enables selective precipitation of the chemically modified strand under denaturing conditions. The concentration of the linear ssDNA, either the sense or antisense strand, is determined by Qubit™ ssDNA Assay Kit according to manufacturer protocols (Thermo Fisher, Q10212).

The complementary strand is created by incubating the ssDNA with a corresponding primer and a PCR mix with unmodified nucleotides. The dsDNA is purified using NucleoSpin® Gel and PCR Clean-Up according to manufacturer protocols (Takara, 740609) and the concentration of the dsDNA is determined by Qubit™ 1×dsDNA Broad Range according to manufacturer protocols (Thermo Fisher, Q33265).

### Example 4: Conversion of Plasmid DNA to Both Sense and Antisense Strand Chemically Modified TDSC

This example describes generation of a TDSC comprising linear dsDNA that comprises chemically modified nucleotides in both the sense and antisense strands. The plasmid construct in Example 1 is converted into chemically modified linear dsDNA by mixing 5-methylcytosine (5mC) with dATP, dGTP, and dTTPs to create the dNTP mix for PCR. The dNTP mix is used with the PrimeSTAR® Max DNA Polymerase according to manufacturer protocols (Takara, R045Q). The dsDNA is purified using NucleoSpin® Gel and PCR Clean-Up according to manufacturer protocols (Takara, 740609) and the concentration of the dsDNA is determined by Qubit™ 1×dsDNA Broad Range according to manufacturer protocols (Thermo Fisher, Q33265).

### Example 5: Addition of an Exonuclease-Resistant DNA End Form Comprising Chemically Modified Nucleotides to the Ends of a TDSC

This example describes the creation of a TDSC comprising linear dsDNA in Examples 2-4 comprising chemically modified nucleotides at both ends of the construct.

A custom adaptor with the following sequence is designed to include the chemically modified nucleotides: 5′-C*C*C*GAGGCGGUACGAGCCACACGTACTACGCTCGTACCGCCUC*G*G*GT-3′ (SEQ ID NO: 85) (* indicates a phosphorothioate bond, bold nucleotides are complementary). The linear dsDNA from any of Examples 2-4 is treated with the NEBNext® Ultra™ II End Repair/dA-Tailing Module according to manufacturer protocols (New England Biolabs, E7546). This kit is used to add a non-templated adenine to the 3′ end of both the sense and antisense strands in the linear dsDNA. Next, the custom adaptor is ligated to the A-tailed dsDNA using the NEBNext® Ultra™ II Ligation Module according to manufacturer protocols (New England Biolabs, E7595). To remove dsDNA constructs that do not have adaptor molecules at each end, the dsDNA is treated with Exonuclease VIII, truncated according to manufacturer protocols (New England Biolabs, M0545S). To remove remaining pieces of the adaptor that do not contain phosphorothioate bonds, the dsDNA is incubated with USER® Enzyme according to manufacturer protocols to remove uracil nucleotides in the adaptor (New England Biolabs, M5505). Then the dsDNA is treated with Mung Bean Nuclease to remove single-stranded DNA overhang from the adaptor and create a TDSC comprising blunt ended dsDNA with phosphorothioate modifications at the ends (New England Biolabs, M0250).

The dsDNA is purified using NucleoSpin® Gel and PCR Clean-Up according to manufacturer protocols (Takara, 740609) and the concentration of the dsDNA is determined by Qubit™ 1×dsDNA Broad Range according to manufacturer protocols (Thermo Fisher, Q33265).

### Example 6: Addition of an Exonuclease-Resistant DNA End Form Comprising a Loop Structure to the Ends of a TDSC

This example describes the creation of a TDSC comprising linear dsDNA in Examples 2-4 with a loop structure at each end of the construct.

A custom adaptor with the following sequence is designed to include the chemically modified nucleotides: 5′-CCCGGGCGGAAGAGCCACACGTACTACGCTCTTCCGCCCGGGT-3′ (SEQ ID NO: 93) (bold nucleotides are complementary). The linear dsDNA from any of Examples 2-4 is treated with the NEBNext® Ultra™ II End Repair/dA-Tailing Module according to manufacturer protocols (New England Biolabs, E7546). This kit is used to add a non-templated adenine to the 3′ end of both the sense and antisense strands in the linear dsDNA. Next, the custom adaptor is ligated to the A-tailed dsDNA using the NEBNext® Ultra™ II Ligation Module according to manufacturer protocols (New England Biolabs, E7595). To remove dsDNA constructs that do not have adaptor molecules at each end, the dsDNA is treated with Exonuclease VIII, truncated according to manufacturer protocols (New England Biolabs, M0545S).

The dsDNA is purified using NucleoSpin® Gel and PCR Clean-Up according to manufacturer protocols (Takara, 740609) and the concentration of the dsDNA is determined by Qubit™ 1×dsDNA Broad Range according to manufacturer protocols (Thermo Fisher, Q33265).

### Example 7: Addition of an Exonuclease-Resistant DNA End Form Comprising a Y-Adaptor to the Ends of a TDSC

This example describes the creation of TDSC comprising linear dsDNA in any of Examples 2-4 with a Y-adaptor at each end of the construct.

A custom adaptor with the following sequence is designed to include the chemically modified nucleotides: 5′-CCCGGGCGGA*C*A*G*C*A*CUC*A*C*G*A*C*TCCGCCCGGGT-3′ (SEQ ID NO: 89) (* indicates a phosphorothioate bond, bold nucleotides are complementary). The linear dsDNA from any of Examples 2-4 is treated with the NEBNext® Ultra™ II End Repair/dA-Tailing Module according to manufacturer protocols (New England Biolabs, E7546). This kit is used to add a non-templated adenine to the 3′ end of both the sense and antisense strands in the linear dsDNA. Next, the custom adaptor is ligated to the A-tailed dsDNA using the NEBNext® Ultra™ II Ligation Module according to manufacturer protocols (New England Biolabs, E7595). To remove dsDNA constructs that do not have adaptor molecules at each end, the dsDNA is treated with Exonuclease VIII, truncated according to manufacturer protocols (New England Biolabs, M0545S). To convert the loop into linear ssDNA strands, the dsDNA is incubated with USER® Enzyme according to manufacturer protocols to remove uracil nucleotides in the adaptor (New England Biolabs, M5505).

The dsDNA is purified using NucleoSpin® Gel and PCR Clean-Up according to manufacturer protocols (Takara, 740609) and the concentration of the dsDNA is determined by Qubit™ 1×dsDNA Broad Range according to manufacturer protocols (Thermo Fisher, Q33265).

### Example 8: Formulation of TDSC with LNP

This example describes how to formulate the constructs made as described in the previous examples with a lipid nanoparticle (LNP).

Nucleic acid constructs are combined with lipid components via microfluidic devices according to the method of Chen et al. 2012. J Am Chem Soc. Volume 134, Issue 16:6948-6951. Briefly, the microfluidic devices are fabricated in polydimethylsiloxane (PDMS) according to standard lithographic procedures (McDonald & Whitesides. 2002. Accounts Chem Res Volume 35, Issue 7:491-499). The lipid components, typically containing cationic lipids, cholesterol, helper lipids, polyethylene glycol modified lipids, and lipids facilitating targeting moiety conjugation (optional), are combined and solubilized in 90% ethanol. The nucleic acid constructs are dissolved in buffer. The nucleic acid solution, the lipid solution, and phosphate buffer saline (PBS) are injected into the microfluidic device. The freshly prepared LNPs are dialyzed against PBS buffer using membranes with MWCO of 3.5 kD to remove ethanol and exchange buffer.

The LNPs are characterized in terms of effective diameter, polydispersity, and zeta potential using dynamic light scattering (DLS) (ZetaPALS, Brookhaven Instruments, NY, 15-mW laser, incident beam 676 nm); and total nucleic acid concentration is determined by lysing the particles and using Quant-iT™ 1×dsDNA Assay Kits, High Sensitivity (HS) and Broad Range (BR) according to the manufacturer protocols (ThermoFisher Scientific, Q33232).

### Example 9: Assessment of Expression and Innate Immune Response in Cells In Vitro

This example describes how to test gene expression, as well as how to determine a TDSC's effect on the innate immune response of cultured cells.

Experimental TDSC constructs are prepared as in Examples 2-7 above. The constructs and controls are administered via electroporation at multiple concentrations to cells selected from HEK, keratinocytes, macrophages, T cells and epithelial cells. An untreated control sample may be run in parallel. After electroporation the cells are moved to the final culture vessels. Constructs formulated with LNPs are directly administered to the cells in well plates.

To determine expression of constructs encoding the fluorescent reporter mCherry, cells are first washed with PBS before flow cytometric analysis. All flow cytometry is performed on MACSQuant VYB by Miltenyi. For detection of mCherry signal, a yellow laser (wavelength 561 nm) is used for excitation and a 615/620 nm emission filter is used. 20,000 events are recorded for each sample and data is analyzed using Flowjo V.9.0 software. Cells are first gated on FSC-A and SSC-A plot to remove cell debris. The population is further plotted on an FSC-A and FSC-H plot to circumscribe the single cell population. Finally, a bivariate plot between the fluorescent signal expressing and non-expressing cells is used to determine the percentage of expressing cells. A distribution of expressing cells is used to determine the level of expression within each cell. Expression analysis is performed at multiple time points.

qPCR is performed on cells to determine the RNA level of IFN-b in the test cells as described in Jakobsen et al. 2013. Proc Natl Acad Sci USA Volume 110, Issue 48: E4571-80. Briefly, the probe-primer sets used in qPCR are human IFN-b (ThermoFisher, Hs01077958_s1) and b-actin (ThermoFisher, Hs00357333_g1). The analyses are performed using pre-made Taqman assays and the RNA-to-Ct one step kit (Applied Biosystems). qPCR is performed on an MX3005 system (Stratagene). RNA expression is normalized to b-actin and to the relevant untreated control. Data are stated as the mean±SEM from biological replicates. ELISA is performed on cell supernatants according to the manufacturer protocol to determine the secreted level of IFN-b.

### Example 10. Determining Exonuclease Resistance for a TDSC Comprising Closed Ends

This example describes how to test if a TDSC comprising closed ends (e.g., an adaptor-ligated linear dsDNA construct) is Exonuclease III (M0206, New England Biolabs Inc.) resistant. The TDSC is tested next to a non-nuclease control. The non-nuclease control contains DNA with the identical sequence to the TDSC of interest except that it underwent the adaptor ligation protocol that is used to add the exonuclease-resistant DNA end form to the TDSC, but without an adaptor oligonucleotide added to the mixture. 1 μL of Exonuclease III (at a starting concentration of 100 units/uL) is added per 5 μg of DNA in 50 μL. The tubes are mixed well and spun down. The tubes are run on the thermocycler for 1 hour at 37° C., and heat inactivated at 70° C. for 30 minutes.

The samples are purified via the Nucleospin® Gel and PCR Clean-up kit (catalog #740609, Macherey-Nagel) using a vacuum manifold according to manufacturer protocols. Briefly, the elution buffer is warmed to 70° C. 2× volumes of NTI binding buffer are added to 1× volume of Exo III-treated DNA. The samples are mixed until evenly distributed and left at room temperature for 5 minutes. The column on the vacuum manifold is secured, valve opened, and vacuum turned on. 375 μL DNA-NTI mix is added to 2× columns and allowed to fully pass through each column. 700 μL of NTC wash buffer is added twice. The column is removed from the vacuum manifold and placed into a collection tube. The assembly is centrifuged at 11,000×g for 1 minute. The column is placed into a new low bind microcentrifuge tube, 25 μL of prewarmed buffer is added, and the assembly is incubated at 70° C. for 5 min. The assembly is centrifuged at 11,000×g for 1 min. The incubation and elution steps are repeated a second time. The collected DNA is quantified by dsDNA BR Qubit (Q32850, Thermo Fisher Scientific) on the Qubit 4 Fluorometer (Q33226, Thermo Fisher Scientific) according to manufacturer protocols.

The samples are loaded into E-Gel EX, 1% Agarose Gel (G402021, Thermo Fisher Scientific) in individual wells at an amount of 16 ng of DNA per well. The ladder (10488090, Thermo Fisher Scientific) is loaded at 2 μl into the left most lane of the gel. The gel is run through the E-Gel Power Snap Electrophoresis System according to manufacturer protocols (G8100, G8200, Thermo Fisher Scientific). After the gel is run, the exonuclease-resistant TDSC is visible at the molecular weight corresponding to the full-length DNA plus closed-adapter sequence. A TDSC will be considered exonuclease-resistant in this assay if at least 95% of the product that appears in the gel in that lane corresponds to the full-length TDSC.

### Example 11. Determining Exonuclease Resistance for a TDSC Comprising an Open End (e.g., Two Open Ends)

This example describes how to test if a TDSC comprising an open end (e.g., an adaptor-ligated linear dsDNA construct) is Exonuclease III (M0206, New England Biolabs Inc.) resistant. The TDSC is tested next to a non-nuclease control. The non-nuclease control contains DNA with the identical sequence to the TDSC of interest except that it underwent the adaptor ligation protocol that is used to add the exonuclease-resistant DNA end form to the TDSC, but without an adaptor oligonucleotide added to the mixture. 2 units of Exonuclease III are added per 200 ng of DNA (at 10 ng/ul), in a 20 μl reaction. The tubes are mixed well and spun down. The tubes are run on the thermocycler for 30 min at 37° C.

The samples are loaded into E-Gel EX, 1% Agarose Gel (G402021, Thermo Fisher Scientific) in individual wells at an amount of 20 ng of DNA per well. The ladder (10488090, Thermo Fisher Scientific) is loaded at 2 μl into the left most lane of the gel. The gel is run through the E-Gel Power Snap Electrophoresis System according to manufacturer protocols (G8100, G8200, Thermo Fisher Scientific). After the gel is run, the exonuclease-resistant TDSC is visible at the molecular weight corresponding to the full-length DNA plus closed-adapter sequence. A TDSC will be considered exonuclease-resistant in this assay if at least 95% of the product that appears in the gel in that lane corresponds to the full-length TDSC.

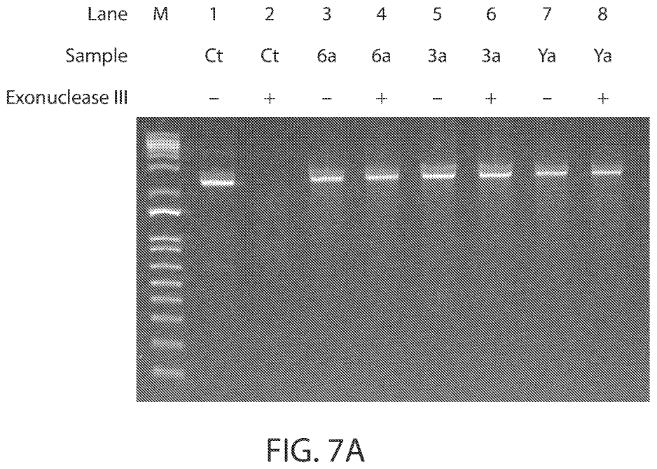

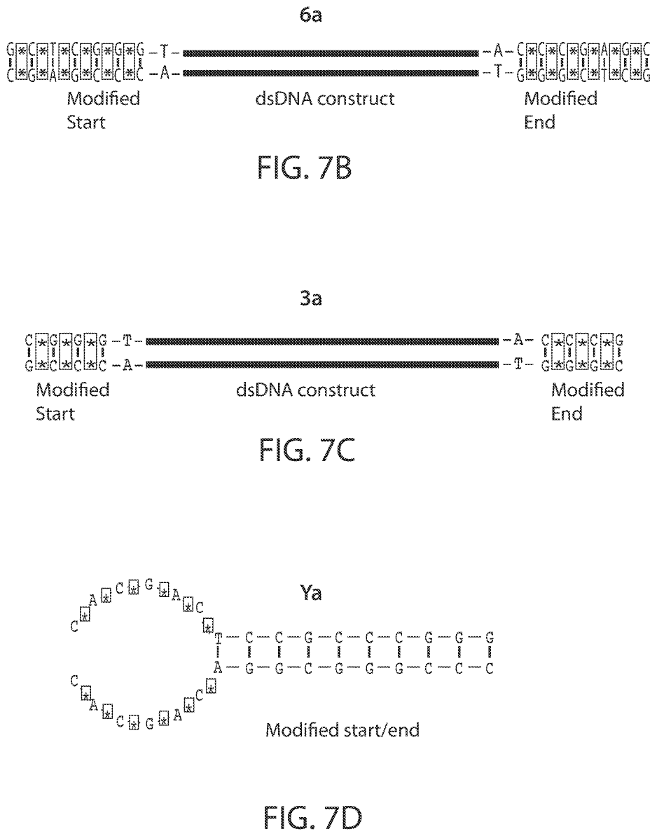

The Exonuclease III digestion protocol in this Example was performed on four samples: a control (unmodified) TDSC designated “Ct”; a TDSC designated “6a” comprising 6 phosphorothioate bonds in each strand at each of the 5′ and 3′ ends (illustrated in FIG. 7B); a TDSC designated “3a” comprising 3 phosphorothioate bonds in each strand at each of the 5′ and 3′ ends (illustrated in FIG. 7C); and a TDSC designated “Ya” comprising identical Y-adapters at each end, each Y-adapter comprising 6 phosphorothioate bonds at the end of each strand (illustrated in FIG. 7D). As shown in FIG. 7A, the control DNA “Ct” was digested, while the three phosphorothioate-modified TDSCs were resistant to exonuclease III digestion.

### Example 12: Manufacturing a TDSC Comprising an Open End

This example describes how to produce a TDSC comprising an open end (e.g., comprising a phosphorothioate linkage), using USER (M5505, New England BioLabs) and Mung Bean Nuclease (M0250, New England BioLabs) treatment steps. This process begins with a proto-TDSC comprising closed ends. The proto-TDSC is processed next to a non-nuclease control. The non-nuclease control contains DNA with the identical sequence to the TDSC except that it underwent the adaptor ligation protocol that is used to add the exonuclease-resistant DNA end form to the TDSC, but without an adaptor oligonucleotide added to the mixture.

These proto-TDSC samples are first processed through the steps to confirm Exonuclease III resistance as described in Example 10. 3 μL of USER enzyme is added to 5 μg of DNA from the purified samples from Example 10 in 100 μL. By removing a uracil positioned in the loops, the USER treatment opens the closed end to form a Y-adaptor like structure. The samples are incubated for 1 hour at 37° C. 1 μL of Mung Bean Nuclease (10 U/uL) is added to each tube. The sample is incubated for 30 min at 30° C. Mung Bean Nuclease degrades the single stranded DNA, yielding a blunt ended TDSC.

The samples are purified via the Nucleospin® Gel and PCR Clean-up kit (740609, Macherey-Nagel) using a vacuum manifold according to manufacturer protocols. Briefly, the elution buffer is warmed to 70° C. 2× volumes of NTI binding buffer is added to 1× volume of USER/MBN-treated DNA. The samples are mixed until evenly distributed and left at room temperature for 5 minutes. The column on the vacuum manifold is secured, valve opened, and vacuum turned on. The DNA-NTI mix is added to column and allowed to fully pass through each column. 700 μL of NTC wash buffer is added twice. The column is removed from the vacuum manifold and placed into a collection tube. The assembly is centrifuged at 11,000×g for 1 minute. The column is placed into a new low bind tube, 25 μL of prewarmed buffer is added, and assembly is incubated at 70° C. for 5 min. The assembly is centrifuged at 11,000×g for 1 min. The incubation and elution steps are repeated a second time. The collected DNA is quantified by dsDNA BR Qubit (Q32850, Thermo Fisher Scientific) on the Qubit 4 Fluorometer (Q33226, Thermo Fisher Scientific) according to manufacturer protocols.

To confirm production of the TDSC, the samples are loaded into and E-Gel EX, 1% Agarose Gel (G402021, Thermo Fisher Scientific) in individual wells at an amount of 16 ng of DNA per well. The ladder (10488090, Thermo Fisher Scientific) is loaded at 2 μl into the left most lane of the gel. The gel is run through the E-Gel Power Snap Electrophoresis System according to manufacturer protocols (G8100, G8200, Thermo Fisher Scientific). After the gel is run, the TDSC is visible at the molecular weight corresponding to the full-length DNA plus adapter sequence.

### Example 13: Manufacturing a TDSC Comprising a Y-Adaptor

This example describes how to produce a TDSC comprising a Y-adaptor end-modified linear dsDNA construct), using USER (M5505, New England BioLabs) to create the open end form. This process begins with a proto-TDSC comprising closed ends. The proto-TDSC is tested next to a non-nuclease control. The non-nuclease control contains DNA with the identical sequence to the TDSC except that it underwent the adaptor ligation protocol that is used to add the exonuclease-resistant DNA end form to the TDSC, but without an adaptor oligonucleotide added to the mixture.

These proto-TDSC samples are first processed through the steps to confirm Exonuclease III resistance as described in Example 10. 3 μL of USER enzyme is added to 5 μg of DNA from the purified samples from Example 10 in 100 μL. The samples are incubated for 1 hour at 37° C. By removing a uracil positioned in the loops, the USER treatment opens the closed end to form a Y-adaptor, yielding the TDSC comprising Y-adaptor end forms.

The samples are purified via the Nucleospin® Gel and PCR Clean-up kit (740609, Macherey-Nagel) using a vacuum manifold according to manufacturer protocols. Briefly, the elution buffer is warmed to 70° C. 2× volumes of NTI binding buffer is added to 1× volume of USER/MBN-treated DNA. The samples are mixed until evenly distributed and left at room temperature for 5 minutes. The column on the vacuum manifold is secured, valve opened, and vacuum turned on. The DNA-NTI mix is added to column and allowed to fully pass through each column. 700 μL of NTC wash buffer is added twice. The column is removed from the vacuum manifold and placed into a collection tube. The assembly is centrifuged at 11,000×g for 1 minute. The column is placed into a new low bind tube, 25 L of prewarmed buffer is added, and assembly is incubated at 70° C. for 5 min. The assembly is centrifuged at 11,000×g for 1 min. The incubation and elution steps are repeated a second time. The collected DNA is quantified by dsDNA BR Qubit (Q32850, Thermo Fisher Scientific) on the Qubit 4 Fluorometer (Q33226, Thermo Fisher Scientific) according to manufacturer protocols.

In order to demonstrate that the final end form is created, a small aliquot of the USER treated DNA is treated with 1 μL of Exonuclease III per 5 μg of DNA in 50 μL of reaction. If the final end form has successfully been created, Exonuclease III will degrade the TDSC of the aliquot.

The samples are loaded into and E-Gel EX, 1% Agarose Gel (G402021, Thermo Fisher Scientific) in individual wells at an amount of 16 ng of DNA per well. The ladder (10488090, Thermo Fisher Scientific) is loaded at 2 μl into the left most lane of the gel. The gel is run through the E-Gel Power Snap Electrophoresis System according to manufacturer protocols (G8100, G8200, Thermo Fisher Scientific). After the gel is run, the Y-adapted TDSC is at the molecular weight corresponding to the full-length DNA plus adapter sequence, while the Exonuclease III treated Y-adapted DNA form is not visible on the gel.

### Example 14: Design and Assembly of a Plasmid Template for Production of Double-Stranded DNA (dsDNA) Molecules

This example describes production of a plasmid template for a dsDNA molecule, e.g., a TDSC. In this example, a construct template was designed with the following specific sequence components.

Promoter Ef1a:
(SEQ ID NO: 37)
5′ggctccggtgcccgtcagtgggcagagcgcacatcgcccacag
tccccgagaagttggggggaggggtcggcaattgaaccggtgcct
agagaaggtggcgcggggtaaactgggaaagtgatgtcgtgtact
ggctccgcctttttcccgaggggggggagaaccgtatataagtgc
agtagtcgccgtgaacgttctttttcgcaacgggtttgccgccag
aacacaggtaagtgccgtgtgtggttcccgcgggcctggcctctt
tacgggttatggcccttgcgtgccttgaattacttccacctggct
gcagtacgtgattcttgatcccgagcttcgggttggaagtgggtg
ggagagttcgaggccttgcgcttaaggagccccttcgcctcgtgc
ttgagttgaggcctggcctgggcgctggggccgccgcgtgcgaat
ctggtggcaccttcgcgcctgtctcgctgctttcgataagtctct
agccatttaaaatttttgatgacctgctgcgacgctttttttctg
gcaagatagtcttgtaaatgcgggccaagatctgcacactggtat
ttcggtttttggggccggggcggcgacggggcccgtgcgtcccag
cgcacatgttcggcgaggcggggcctgcgagcgcggccaccgaga
atcggacgggggtagtctcaagctggccggcctgctctggtgcct
ggcctcgcgccgccgtgtatcgccccgccctgggcggcaaggctg
gcccggtcggcaccagttgcgtgagcggaaagatggccgcttccc
ggccctgctgcagggagctcaaaatggaggacgcggcgctcggga
gagcggggggtgagtcacccacacaaaggaaaagggcctttccgt
cctcagccgtcgcttcatgtgactccacggagtaccgggcgccgt
ccaggcacctcgattagttctcgagcttttggagtacgtcgtctt
taggttggggggaggggttttatgcgatggagtttccccacactg
agtgggtggagactgaagttaggccagcttggcacttgatgtaat
tctccttggaatttgccctttttgagtttggatcttggttcattc
tcaagcctcagacagtggttcaaagtttttttcttccatttcagg
tgtcgtga-3′
Effector sequence encoding a model/marker
protein (mCherry):
(SEQ ID NO: 38)
5′atggtgagcaagggcgaggaggataacatggccatcatcaagg
agttcatgcgcttcaaggtgcacatggagggctccgtgaacggcc
acgagttcgagatcgagggcgagggcgagggccgcccctacgagg
gcacccagaccgccaagctgaaggtgaccaagggggccccctgcc
cttcgcctgggacatcctgtcccctcagttcatgtacggctccaa
ggcctacgtgaagcaccccgccgacatccccgactacttgaagct
gtccttccccgagggcttcaagtgggagcgcgtgatgaacttcga
ggacggcggcgtggtgaccgtgacccaggactcctccctgcagga
cggcgagttcatctacaaggtgaagctgcgcggcaccaacttccc
ctccgacggccccgtaatgcagaagaagaccatgggctgggaggc
ctcctccgagcggatgtaccccgaggacggcgccctgaagggcga
gatcaagcagaggctgaagctgaaggacggcggccactacgacgc
tgaggtcaagaccacctacaaggccaagaagcccgtgcagctgcc
cggcgcctacaacgtcaacatcaagttggacatcacctcccacaa
cgaggactacaccatcgtggaacagtacgaacgcgccgagggccg
ccactccaccggcggcatggacgagctgtacaagtaa-3′

### Optional:

NTS: SV40 enhancer:
(SEQ ID NO: 1)
5′-cccaagaagaagaggaaagtc-3′
Maintenance sequence: human interferon-β MAR
(SEQ ID NO: 39)
5′tataattcactggaatttttttgtgtgtatggtatgacatatg
ggttcccttttattttttacatataaatatatttccctgtttttc
taaaaaagaaaaagatcatcattttcccattgtaaaatgccatat
ttttttcataggtcacttacata3′
Second strand motif: AAV2 wildtype ITR
(SEQ ID NO: 26)
5′aggaacccctagtgatggagttggccactccctctctgcgcgc
tcgctcgctcactgaggccgggcgaccaaaggtcgcccgacgccc
gggctttgcccgggcggcctcagtgagcgagcgagcgcgcagctg
cctgcagg-3′

A plasmid template was designed with these elements using standard DNA design manipulation software. Once designed, plasmids were ordered from a commercial supplier (GenScript) for use as a template in PCR amplification.

### Example 15: Production of TDSCs with Chemical Modifications

This Example demonstrates preparation of double-stranded DNA (dsDNA) molecules, e.g., TDSCs, containing cytosines with chemical modifications, such as 5-formyl-2′-deoxycytosine (5-formylcytosine).

Plasmid DNA (10 ng/50 μl PCR reaction) was used as a template for PCR amplification using KOD polymerase (710864, Sigma Aldrich) or KOD Xtreme (KODX) polymerase (719753, Sigma Aldrich). Other commercially available polymerases may also be used. The product versions used were separated into their constitutive components, rather than in a mastermix format, to ensure precise ratios of modified nucleotides to standard dNTPs. PCR reaction conditions for each enzyme included:
a. For the KOD enzyme, MgSO₄ at a final concentration of 2 mM.
b. 100 mM dNTP solution set (N0446, New England Biolabs), at a final concentration of 200 μM.
c. Modified deoxynucleoside triphosphates (e.g. 5-formyl-dCTP, N-2064, Trilink Biotechnologies) were added at various ratios with their cognate dNTP, summing to a total of 200 μM (i.e., 200 μM dATP, 200 μM dCTP, 200 μM dTTP, and 200 μM dGTP). Thus, a reaction designed for 25% incorporation would be 50 M modified nucleotide and 150 μM unmodified nucleotide.
d. Forward and reverse primers at a final concentration of 300 μM.

For the synthesis of covalently closed TDSCs, primers contained either a phosphate group for improved ligation efficiency or a TelN recognition sequence.

For the synthesis of circular double-stranded DNA forms, in addition to containing sequences complementary to the plasmid, primers contained additional sequences useful in downstream processes:
a. Nicking enzyme(s) recognition sequence;
b. Restriction enzyme recognition sequence (e.g. BsaI, KpnI, or NheI), used to create sticky-ends in the DNA after restriction enzyme digestion and facilitate DNA circularization; and
c. Additional bases (e.g., 5′-CCGTGGTCCTTC-3′) (SEQ ID NO: 40) to increase restriction enzyme digestion efficiency.

For all forms, the PCR product was purified using standard DNA purification columns.

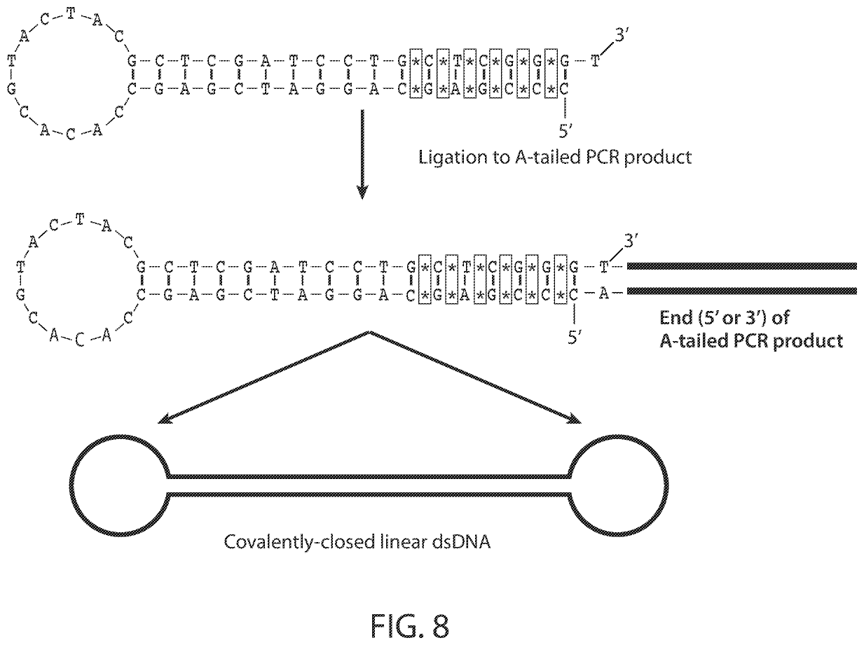

FIG. 8 depicts production of covalently closed TDSCs with end forms comprising phosphorothioate modifications. For TDSCs with end forms comprising phosphorothioate modifications (phosphorothioate end forms), up to 10 μg in 50 μL of PCR DNA per reaction was added to the NEBNext Ultra II End Repair/dA-Tailing buffer (8 μL) and enzyme (3 μL) mixes (E7546L), first at 20° C. for 30 min, then 65° C. for 30 min. After briefly cooling on ice, NEBNext Ultra II ligation module components were added, including ligation mix (30 μL), ligation enhancer (1 μL), and 3 μL of a 100 μM solution containing the DNA adapter to be ligated. This reaction was incubated >1 hr, but typically overnight. The ligated PCR-adapter solution was then purified by Nucleospin Midi columns, quantified by Nanodrop, and any non-ligated PCR was cleaned up with ExoIII (NEB M0206) for one hour at 37° C.

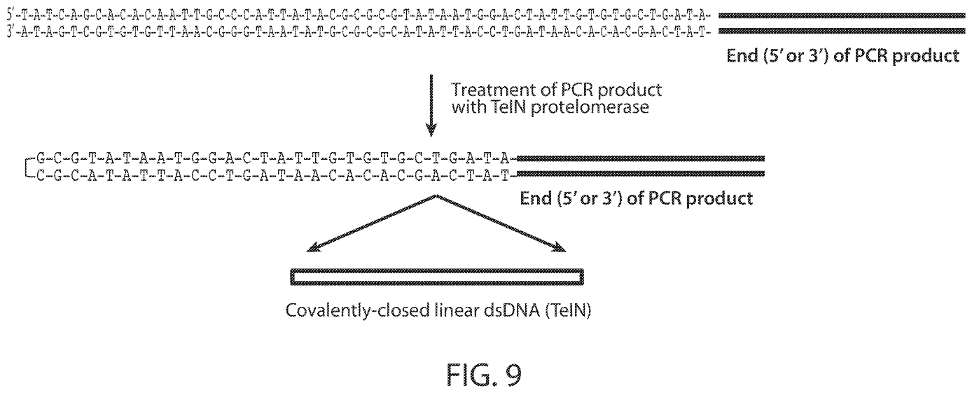

FIG. 9 depicts production of covalently closed TDSCs with TelN end forms. For TDSCs with TelN end forms, 1 μg of PCR DNA was incubated in a 40 μL reaction containing 4 μL 10× ThermoPol buffer, 2 μL TelN protelomerase (M0651, New England Biolabs) at 30° C. for 1 hr. The TelN modified DNA was then purified by Zymo DCC-100 columns, quantified by Nanodrop, and any unmodified PCR was cleaned up with ExoIII (NEB M0206) for one hour at 37° C.

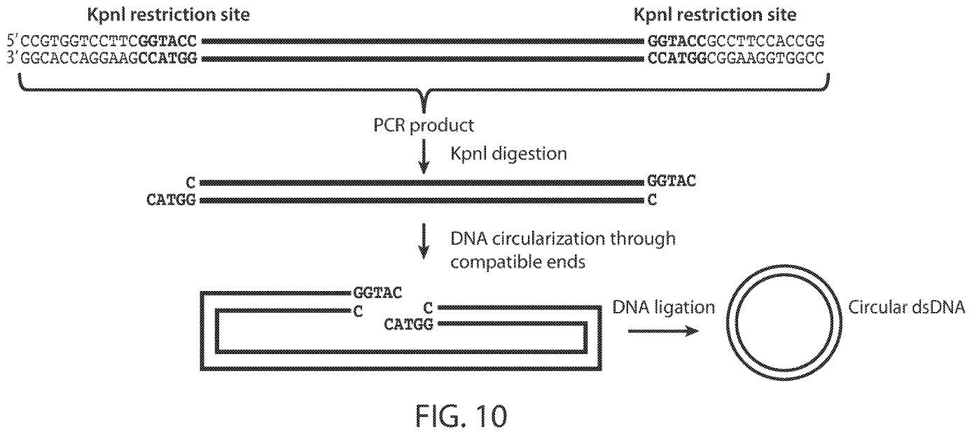

FIG. 10 depicts production of circular dsDNA molecules. For the synthesis of circular dsDNA molecules, DNA was digested, in an overnight reaction, using the restriction enzyme corresponding to the restriction enzyme recognition sequence, for instance, KpnI-HF-V2 (R3142, New England Biolabs). DNA was then purified using DNA purification columns. Digested DNA was circularized using T3 DNA ligase (M0317, New England Biolabs) for one hour at 26° C. Non-circularized DNA was degraded by incubating the DNA with T5 exonuclease (M0663L, New England Biolabs) for one hour at 37° C. T5 exonuclease was used to digest linear dsDNA but not circular dsDNA. DNA was purified using DNA purification columns. Other similar methods may also be used, for instance, agarose gel purification.

Analysis of the composition and purity the resultant double-stranded DNA forms was performed on an Agilent 5300 Fragment Analyzer using the CRISPR Discovery Kit (DNF-930-K1000CP). The dsDNA inlet buffer and running gel with intercalating dye was prepared fresh each day, while the marker tray with mineral oil overlay and capillary conditioning solution were prepared fresh each month. The buffers were prepared to the manufacturer's specifications. Circular double stranded DNA samples were diluted in water to a final concentration of 100 pg/uL. For each sample well, 2 μL of the DNA samples were added to 22 μL of Dilution Buffer (0.1×TE), and each sample was run with 2-4 replicates, with one well used for the MDK DNA ladder. The samples were run via the instrument controller software using default settings of the CRISPR Discovery Method (CRP-910-33).

Sample traces were analyzed using the ProSize Data Analysis Software v4.0.2.7. Peak Analysis conditions for dsDNA were set at the standard conditions of a ‘Peak Width (sec)’ of 5 and a ‘Min. peak height (RFU)’ of 50, #Extra Valley Points of 3, and with ‘Valley to Valley Baseline?’ turned on. Manual baseline was set at-2 min from the lower marker and +2 min to from the upper marker. Peaks were automatically detected by the software under these conditions, and peaks widths were chosen by the software except for instances where manual adjustments were required to due to broad peaks, peak shoulders, or to multiple peaks within a narrow size range.

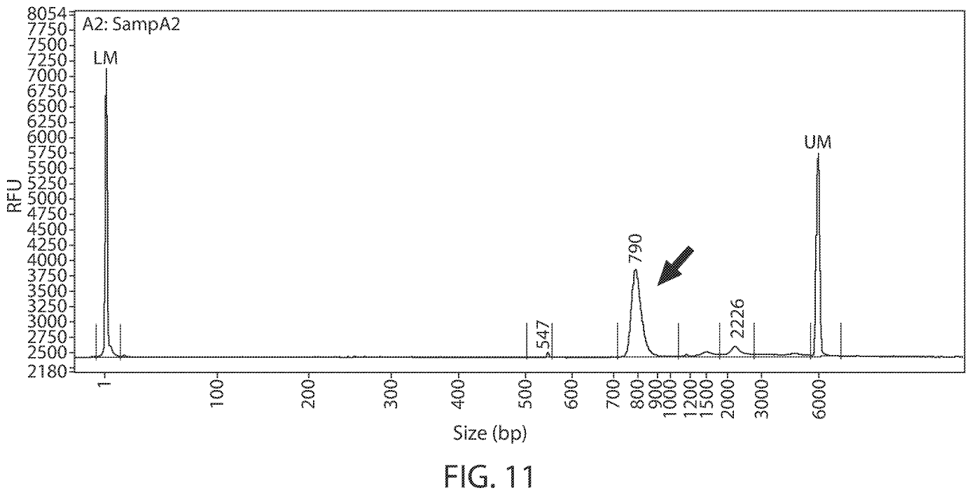

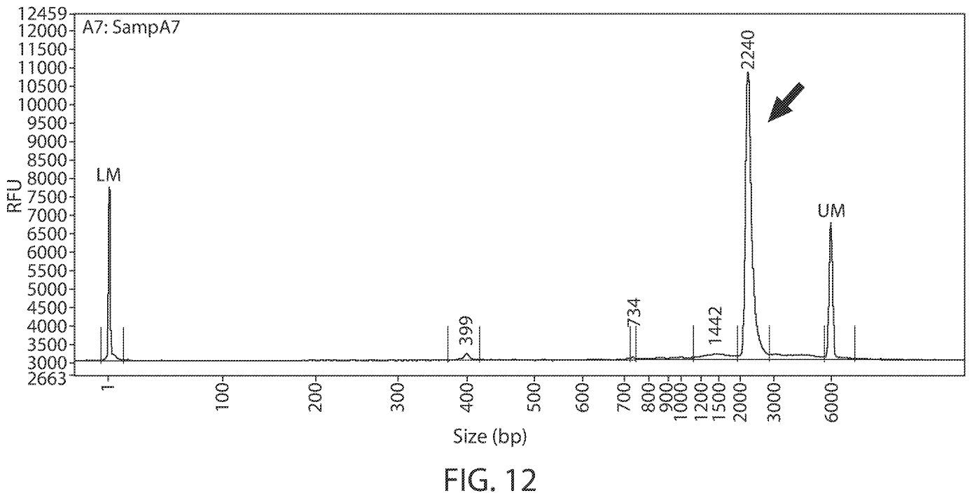

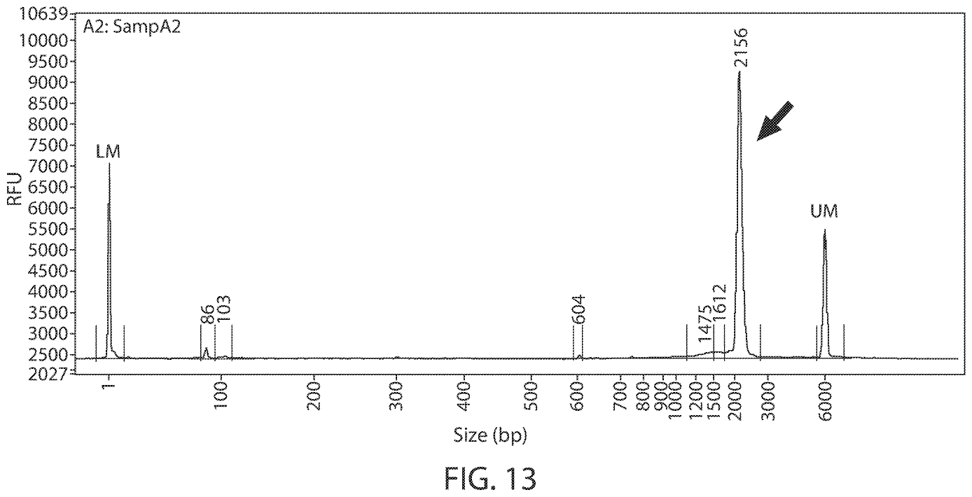

FIGS. 11-13 show fragment analyzer traces of dsDNA forms with 5′ cytosine modifications. FIG. 11 shows a circular dsDNA construct, produced in a reaction using 25% 5-formylcytosine and purified as described above. FIGS. 12-13 show linear covalently closed dsDNA constructs with phosphorothioate end forms (FIG. 12) and TelN end forms (FIG. 13), produced in reactions using 25% 5-formylcytosine and purified as described above. In each trace, a single peak (indicated with an arrow) is clearly visible. These results indicate that multiple TDSC forms, including those comprising chemically modified nucleotides (e.g., 5-formylcytosine), can be produced and purified.

### Example 16: Assessment of TDSC Gene Expression In Vitro

This example demonstrates detection and quantification of gene expression using chemically modified TDSCs in cultured cells.

Experimental constructs and controls were administered via lipid transfection (lipofection). Lipofection for DNA was performed using the Lipofectamine3000 transfection reagent (#L3000001, ThermoFisher) in HEKa cells according to manufacturer's instructions. A 1:2:3 ratio of DNA: P3000: Lipofectamine3000 was used for all DNA constructs and controls. 10,000 cells were pre-seeded into each well of 96-well plates one day before transfection. Transfection was performed when cells reached roughly 80 to 90% confluence. For each well of a 96-well plate, 3× Lipofectamine3000 was first diluted in 5 μL of Opti-MEM™ I Reduced Serum Medium (#31985070, ThermoFisher). DNA was diluted in 5 μL Opti-MEM™ I Reduced Serum Medium with 2×P3000 reagent. The DNA was then added into the Lipofectamine3000 containing Opti-MEM™ I Reduced Serum Medium and mixed gently by pipetting. After incubating for 15 minutes at room temperature, the DNA-Lipofectamine3000 complex was added to target cells with full culture medium in a dropwise manner to different areas of the well. The plate was gently rocked back-and-forth and side-to-side to evenly distribute the DNA-Lipofectamine3000 complex. Following transfection, cells were incubated in a CO₂ tissue culture incubator, and culture medium was changed 6 to 8 hours after transfection.

To determine expression of constructs encoding the fluorescent reporter mCherry, cells were first washed with PBS before flow cytometric analysis. All flow cytometry was performed on MACSQuant VYB by Miltenyi. For detection of mCherry signal, a yellow laser (wavelength 561 nm) was used for excitation and a 615/620 nm emission filter was used. 20,000 events were recorded for each sample and data were analyzed using Flowjo V.9.0 software. Cells were first gated on FSC-A and SSC-A plot to remove cell debris. The population was further plotted on an FSC-A and FSC-H plot to circumscribe the single cell population. Finally, a bivariate plot between the fluorescent signal expressing and non-expressing cells was used to determine the percentage of expressing cells. A distribution of expressing cells was used to determine the level of expression within each cell. Expression analysis was performed at multiple time points.

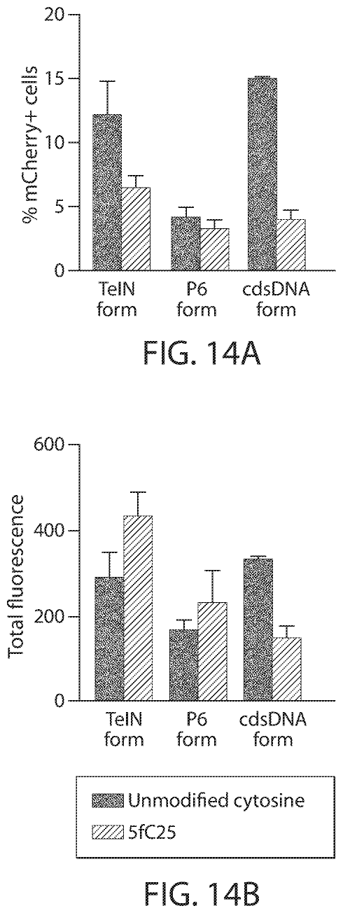

FIGS. 14A-14B show that multiple TDSC constructs, produced with and without chemical modifications, enable expression of a reporter gene. In HEKa cells, dsDNAs with three different structures-circular double-stranded, covalently closed TDSC with phosphorothioate end forms, and covalently closed TDSC with TelN end forms-yielded detectable expression of the reporter protein mCherry. DNA molecules in which deoxycytosine was at least partially replaced with 5-formylcytosine, as described in Example 15, also retained function, as defined by a similar proportion of cells expressing mCherry. These results demonstrate that TDSCs with different end forms can be transcribed and yield a protein product, even when chemically modified.

### Example 17: Assessment of the Effects of TDSCs on Innate Immune Response in Cells In Vitro

This example describes the effect of chemically modified dsDNA constructs, e.g., TDSCs, on the innate immune response of cultured cells.

Experimental constructs were prepared as in Example 15 above, then administered to cells as in Example 16 above. qPCR was performed on cells to determine the RNA level of a panel of proinflammatory cytokines, including human IFNL1, CXCL8, TNF, IL17B, IL6, IFNB1, CCL2, IL23, IL17E, CXCL10, CXCL1, CCL5, IL1B, IL5, IL33, IL1A, CXCL2, IL17C, and IL18. Human GAPDH was used as an endogenous control for analysis. Primer sequences can be found in the attached Table 4. Briefly, mRNA was extracted from cells using the PicoPure RNA Isolation Kit (ThermoFisher #KIT0204) according to the manufacturer's instructions. cDNA was synthesized using the RNA to cDNA EcoDry™ Premix (Oligo dT) (Takara #639542) kit following the manufacturer's instructions. The analyses were performed using the QuantStudio7 Flex Real-time PCR System with SYBR Select Master Mix from Life Technologies Corporation. RNA expression was normalized to GAPDH and expressed as fold-changes relative to the method control (lipofection reagent without DNA).

TABLE 4
Primer sequences used in qPCR quantification of immune markers.

| Gene Name | 5′-Sequence-3′ | SEQ ID NO: |
|---|---|---|
| h-GAPDH-F | GTCTCCTCTGACTTCAACAGCG | 41 |
| h-GAPDH-R | ACCACCCTGTTGCTGTAGCCAA | 42 |
| h-IL6-F | AGACAGCCACTCACCTCTTCAG | 43 |
| h-IL6-R | TTCTGCCAGTGCCTCTTTGCTG | 44 |
| h-CXCL10-F | GGTGAGAAGAGATGTCTGAATCC | 45 |
| h-CXCL10-R | GTCCATCCTTGGAAGCACTGCA | 46 |
| h-IL33-F | GCCTGTCAACAGCAGTCTACTG | 47 |
| h-IL33-R | TGTGCTTAGAGAAGCAAGATACTC | 48 |
| h-IFNL1-F | AACTGGGAAGGGCTGCCACATT | 49 |
| h-IFNL1-R | GGAAGACAGGAGAGCTGCAACT | 50 |
| h-IFNB1-F | CTTGGATTCCTACAAAGAAGCAGC | 51 |
| h-IFNB1-R | TCCTCCTTCTGGAACTGCTGCA | 52 |
| h-CXCL1-F | AGCTTGCCTCAATCCTGCATCC | 53 |
| h-CXCL1-R | TCCTTCAGGAACAGCCACCAGT | 54 |
| h-IL1A-F | TGTATGTGACTGCCCAAGATGAAG | 123 |
| h-IL1A-R | AGAGGAGGTTGGTCTCACTACC | 124 |
| h-CXCL8-F | GAGAGTGATTGAGAGTGGACCAC | 125 |
| h-CXCL8-R | CACAACCCTCTGCACCCAGTTT | 126 |
| h-CCL2-F | AGAATCACCAGCAGCAAGTGTCC | 127 |
| h-CCL2-R | TCCTGAACCCACTTCTGCTTGG | 128 |
| h-CCL5-F | CCTGCTGCTTTGCCTACATTGC | 129 |
| h-CCL5-R | ACACACTTGGCGGTTCTTTCGG | 130 |
| h-CXCL2-F | GGCAGAAAGCTTGTCTCAACCC | 131 |
| h-CXCL2-R | CTCCTTCAGGAACAGCCACCAA | 132 |
| h-TNF-F | CTCTTCTGCCTGCTGCACTTTG | 133 |
| h-TNF-R | ATGGGCTACAGGCTTGTCACTC | 134 |
| h-IL23A-F | GAGCCTTCTCTGCTCCCTGATA | 135 |
| h-IL23 A-R | GACTGAGGCTTGGAATCTGCTG | 136 |
| h-IL 1B-F | CCACAGACCTTCCAGGAGAATG | 137 |
| h-IL1B-R | GTGCAGTTCAGTGATCGTACAGG | 138 |
| h-IL17C-F | GCCCTCAGCTACGACCCAGTG | 139 |
| h-IL17C-R | AGCTTCTGTGGATAGCGGTCCT | 140 |
| h-IL 17B-F | GCTGTGGATGTCCAACAAGAGG | 141 |
| h-IL17B-R | TCCTGCATGGTGAAGGGGTTCA | 142 |
| h-IL17E-F | AACCGCCACCCAGAGTCCTGT | 143 |
| h-IL 17E-R | ACAGGCAACGGGCGTGGTACA | 144 |
| h-IL5-F | GGAATAGGCACACTGGAGAGTC | 145 |
| h-IL5-R | CTCTCCGTCTTTCTTCTCCACAC | 146 |
| h-IL18-F | GATAGCCAGCCTAGAGGTATGG | 147 |
| h-IL18-R | CCTTGATGTTATCAGGAGGATTCA | 148 |

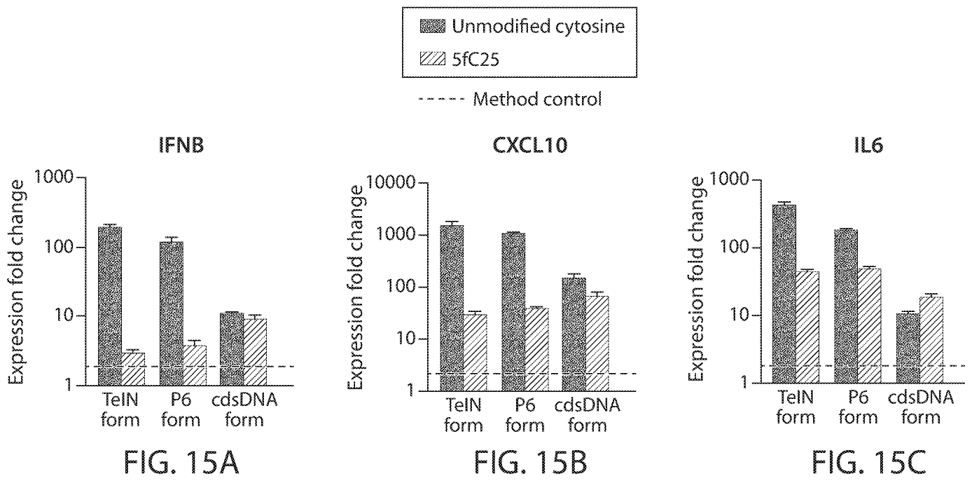

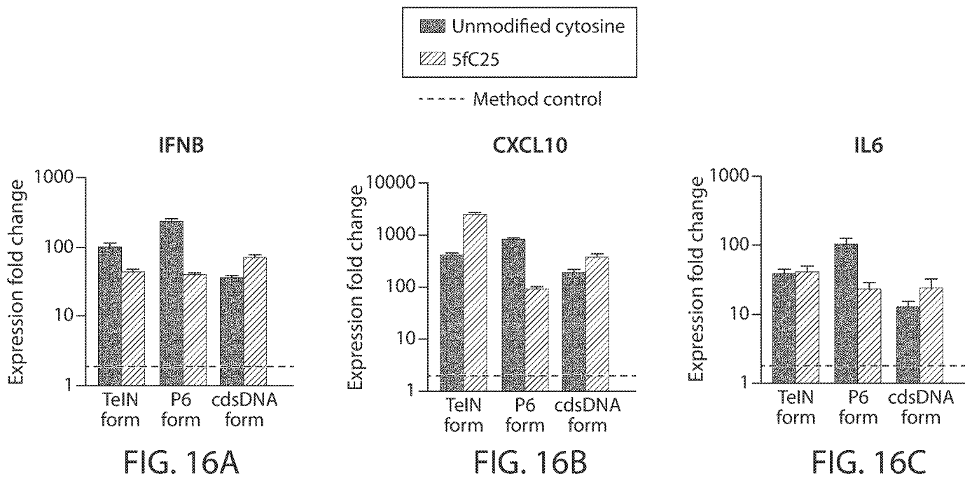

FIGS. 15A-15C and 16A-16C show the innate immune response of HEKa (FIGS. 15A-15C) and THP1 cells (FIGS. 16A-16C) to TDSCs, produced with or without 5-formylcytosine. In both HEKa and THP1 cells, dsDNAs with three different structures-circular double-stranded, linear TDSC with phosphorothioate end forms, and linear TDSC with TelN end forms—yielded measurable innate immune responses, as defined by detectable expression of the cytokines IFNB, CXCL10, and IL6. For covalently closed TDSCs, partial substitution of cytosine with 5-formylcytosine reduced immune responses in both HEKa and THP1 cells, as defined by a reduction in IFNB, CXCL10, and IL6 expression. These results demonstrate that both end form structure of the TDSCs and the presence of chemical modifications (e.g., 5-formylcytosine) can affect the innate immune response to double-stranded DNA while retaining the capacity to encode a functional protein product.

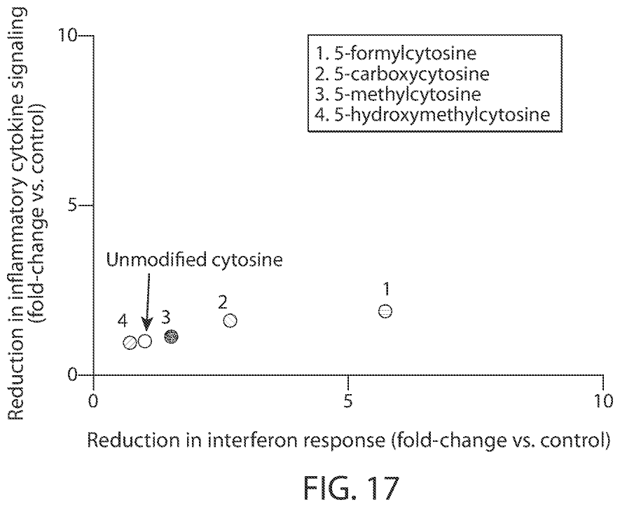

FIG. 17 shows the innate immune response of HEKa cells to covalently closed TDSCs with phosphorothioated end adapters and comprising various modifications at the carbon 5 (C-5) position of cytosine. For each distinct chemically modified dsDNA molecule, the innate immune response was visualized as a scatter plot in which the X-axis represents reduction in interferon signaling, defined as the average fold-change reduction of markers IFNB and CXCL10 relative to a TDSC comprising unmodified cytosines, and in which the Y-axis represents reduction in inflammatory cytokine signaling, defined as the average fold-change reduction of markers IL6 and TNFa relative to a TDSC comprising unmodified cytosines. These results demonstrate that incorporation of specific chemical modifications at the C-5 position of cytosine of TDSCs can reduce the innate immune response to dsDNA in an immune-competent cell line.

All publications, patents, and patent applications cited herein are incorporated by reference in their entirety to the same extent as if each individual publication, patent, or patent application was specifically and individually indicated to be incorporated by reference. In the event of a conflict between a term herein and a term in an incorporated reference, the term herein controls.

1. A TDSC comprising:
a) an upstream DNA end form which is a closed end;
b) a double stranded region;
c) a downstream DNA end form which is a closed end,
wherein the TDSC comprises one or more chemically modified nucleotides,
wherein one or more chemically modified nucleotides comprise a chemically modified cytosine nucleotide and/or a phosphorothioate bond.

2. The TDSC of claim 1, wherein one or both of the upstream DNA end form and downstream DNA end form comprise a loop.

3. The TDSC of claim 1 or 2, wherein the upstream DNA end form, the downstream DNA end form, and/or the double-stranded region comprise one or more chemically modified nucleotides.

4. The TDSC of any of claims 1-3, wherein one or more of the chemically modified nucleotides is conjugated to a peptide or protein.

5. The TDSC of any of claims 1-4, wherein one or more of the chemically modified nucleotides comprises a phosphorothioate bond.

6. The TDSC of any of claims 1-5, wherein each of the first and second strands of the TDSC comprises one or more chemically modified nucleotides.

7. The TDSC of any of claims 1-6, wherein each of the first and second strands of the TDSC comprises one or more phosphorothioate bonds.

8. The TDSC of any of claims 1-7, wherein the upstream and/or downstream DNA end form each comprises at least 1, 2, 3, 4, 5, or 6 phosphorothioate bonds (e.g., between the 1, 2, 3, 4, 5, 6, 7, 8, 9, or 10 terminal nucleotides of the upstream and downstream DNA end forms, e.g., on the first strand, the second strand, or both of the first and second strands).

9. The TDSC of any of claims 1-8, wherein one or more chemically modified nucleotides comprises a chemically modified cytosine nucleotide.

10. The TDSC of claim 9, wherein the chemically modified cytosine nucleotide has a substitution other than hydrogen at carbon 5 of the cytosine.

11. The TDSC of any of claims 1-10, which comprises one or more of:
i) a promoter sequence (wherein optionally the promoter sequence is in the double stranded region);
ii) a payload sequence (e.g., a therapeutic payload sequence) operably linked to the promoter sequence (wherein optionally the payload sequence is in the double stranded region);
iii) a heterologous functional sequence, e.g., a nuclear targeting sequence or a regulatory sequence;
iv) a maintenance sequence; and/or
v) an origin of replication.

12. The TDSC of claim 11, wherein the payload sequence encodes a polypeptide (e.g., a protein).

13. The TDSC of claim 11 or 12, wherein the payload sequence encodes a functional RNA (e.g., a miRNA, siRNA, or tRNA).

14. The TDSC of any of claims 11-13, wherein the payload sequence is heterologous to a target cell.

15. The TDSC of any of claims 1-14, wherein the TDSC is resistant to endonuclease digestion and/or resistant to immune sensor recognition.

16. The TDSC of any of claims 1-15, wherein the upstream DNA end form and the downstream DNA end form have the same nucleotide sequence.

17. The TDSC of any of claims 1-15, wherein the upstream DNA end form and the downstream DNA end form have different nucleotide sequences.

18. The TDSC of any of claims 1-17, wherein one or both of the upstream exonuclease-resistant DNA end form and downstream exonuclease-resistant DNA end form comprise a hairpin.

19. The TDSC of any of claims 1-18, wherein the closed end comprises one or more (e.g., at least 1, 2, 3, 4, 5, 6, 7, 8, 9, 10, 15, 20, 25, 30, 40, or 50) nucleotides that are not hybridized (e.g., are not part of a double-stranded region).

20. The TDSC of any of claims 1-17, wherein the closed end does not comprise any nucleotides that are not hybridized (e.g., wherein all nucleotides of the closed end are hybridized to another nucleotide).

21. The TDSC of any of claims 1-20, wherein the upstream DNA end form, the downstream DNA end form, or both, comprises a protelomerase sequence.

22. The TDSC of any of claims 1-20, wherein the upstream DNA end form, the downstream DNA end form, or both, does not comprise a protelomerase sequence.

23. The TDSC of any of claims 1-22, wherein 80%, 85%, 90%, 95%, 98%, 99%, or 100% of the sugars of the TDSC are deoxyribose sugars.

24. The TDSC of any of claims 1-23, wherein the TDSC can be replicated (e.g., by a DNA polymerase native to a cell comprising the TDSC).

25. The TDSC of any of claims 1-23, wherein the TDSC cannot be replicated.

26. The TDSC of any of claims 1-25, wherein the TDSC is linear and can be circularized.

27. The TDSC of any of claims 1-25, wherein the TDSC is linear and cannot be circularized.

28. The TDSC of any of claims 1-27, wherein the TDSC or a portion thereof can be integrated into the genome.

29. The TDSC of any of claims 1-27, wherein the TDSC or a portion thereof cannot be integrated into the genome.

30. The TDSC of any of claims 1-29, wherein the TDSC can be concatemerized.

31. The TDSC of any of claims 1-29, wherein the TDSC cannot be concatemerized.

32. A TDSC comprising:
a) an upstream exonuclease-resistant DNA end form;
b) a double stranded region; and
c) a downstream exonuclease-resistant DNA end form,
wherein the TDSC comprises one or more chemically modified nucleotides.

33. The TDSC of claim 32, wherein one or both of the upstream exonuclease-resistant DNA end form and downstream exonuclease-resistant DNA end form are open ends.

34. The TDSC of claim 32 or 33, wherein one or both of the upstream exonuclease-resistant DNA end form and downstream exonuclease-resistant DNA end form are blunt ends or sticky ends.

35. A TDSC comprising:
a) an upstream double stranded, blunt-ended DNA end form (e.g., an upstream exonuclease-resistant DNA end form that is double stranded and blunt-ended) comprising a phosphorothioate modification on each strand;
b) a double stranded region; and
c) a downstream double stranded, blunt-ended DNA end form (e.g., a downstream exonuclease-resistant DNA end form that is double stranded and blunt-ended) comprising a phosphorothioate modification on each strand.

36. A TDSC comprising:
a) an upstream exonuclease-resistant DNA end form;
b) a double stranded region;
c) a downstream exonuclease-resistant DNA end form,
wherein one or both of the upstream exonuclease-resistant DNA end form and the downstream exonuclease-resistant DNA end form comprises a Y-adaptor configuration,
optionally wherein the TDSC comprises one or more chemically modified nucleotides.

37. A TDSC comprising:
a) an upstream exonuclease-resistant DNA end form;
b) a double stranded region;
c) a downstream exonuclease-resistant DNA end form,
wherein one or both of the upstream exonuclease-resistant DNA end form and the downstream exonuclease-resistant DNA end form comprises one or more of: a nuclear targeting sequence, a maintenance sequence, or a sequence that binds an endogenous polypeptide in a target cell.

38. A TDSC comprising:
a) an upstream exonuclease-resistant DNA end form;
b) a double stranded region;
c) a downstream exonuclease-resistant DNA end form,
wherein one or both of the upstream exonuclease-resistant DNA end form and the downstream exonuclease-resistant DNA end form have one or more of the following characteristics:
i) does not comprise the nucleic acid sequences TATCAGCACACAATTGCCCATTATACGC (SEQ ID NO: 55) and GCGTATAATGGGCAATTGTGTGCTGATA (SEQ ID NO: 56), or nucleic acid sequences having at least 75%, 80%, 85%, 90%, 95%, 96%, 97%, 98%, or 99% sequence identity thereto; and/or the nucleic acid sequences TATCAGCACACAATAGTCCATTATACGC (SEQ ID NO: 57) and GCGTATAATGGACTATTGTGTGCTGATA (SEQ ID NO: 58);
ii) every nucleotide in the TDSC binds another nucleotide in the TDSC;
iii) the upstream exonuclease-resistant DNA end form has a loop size of less than about 28 or 56 nucleotides in length or greater than about 28 or 56 nucleotides in length; or
iv) the downstream exonuclease-resistant DNA end form has a loop size of less than about 28 or 56 nucleotides in length or greater than about 28 or 56 nucleotides in length.

39. A pharmaceutical composition comprising a double stranded DNA (dsDNA) comprising an effector sequence, wherein:
a) the dsDNA lacks a vector backbone or lacks a material portion of vector backbone, or does not comprise a non-human (e.g., bacterial) origin of replication;
b) the dsDNA is unencapsidated, is essentially free of viral proteins, does not comprise a viral packaging signal, or does not comprise a viral ITR;
c) the dsDNA comprises exonuclease-resistant ends; and
d) the dsDNA comprises at least one chemically modified nucleotide.

40. A pharmaceutical composition comprising a TDSC of any of claims 1-38.

41. The pharmaceutical composition of claim 39 or 40, wherein the dsDNA or TDSC is comprised in a lipid nanoparticle (LNP).

42. A proto-TDSC comprising:
a) an upstream exonuclease-resistant DNA end form;
b) a double stranded region;
c) a downstream exonuclease-resistant DNA end form,
wherein:
(i) the proto-TDSC comprises one or more (e.g., 1 or 2) uracil nucleotides, or
(ii) the upstream exonuclease-resistant DNA end form comprises a sticky end, and/or wherein the downstream exonuclease-resistant DNA end form comprises a sticky end.

43. A method of expressing a heterologous payload in a target cell, the method comprising:
(i) introducing into a target cell a TDSC or composition of any of claims 1-41, wherein the double-stranded region of the TDSC comprises a sequence encoding a heterologous payload; and
(ii) maintaining (e.g., incubating) the cell under conditions suitable for expressing the heterologous payload from the TDSC;
thereby expressing the heterologous payload in the target cell.

44. A method of delivering a heterologous payload to a target cell, the method comprising:
introducing into a target cell a TDSC or composition of any of claims 1-41, wherein the double-stranded region of the TDSC comprises a sequence encoding a heterologous payload;
thereby delivering the heterologous payload to the target cell.

45. A method of modulating (e.g., increasing or decreasing) a biological activity in a target cell, the method comprising:
(i) introducing into a target cell a TDSC or composition of any of claims 1-41, wherein the double-stranded region of the TDSC comprises a sequence encoding a heterologous payload that modulates a biological activity in the target cell; and
(ii) maintaining (e.g., incubating) the cell under conditions suitable for expressing the heterologous payload from the TDSC;
thereby modulating the biological activity in the target cell.

46. A method of treating a cell, tissue, or subject in need thereof, the method comprising:
administering to the cell, tissue, or subject a TDSC or composition of any of claims 1-41, wherein the double-stranded region of the TDSC comprises a sequence encoding a heterologous payload;
thereby treating the cell, tissue, or subject.

47. A method of making a TDSC, the method comprising ligating:
a double-stranded DNA molecule to
a self-annealed DNA molecule comprising a first region and a second region, wherein the first region is hybridized to the second region;
thereby producing a TDSC,
optionally wherein the self-annealed DNA molecule further comprises a loop between the first region and the second region.


SEQUENCE LISTING (148 sequences)

SEQ ID NO: 1 - DNA, 21 bp, Simian virus 40
  source 1..21: mol_type = genomic DNA
cccaagaagaagaggaaagtc

SEQ ID NO: 2-25 - skipped (24 entries)

SEQ ID NO: 26 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27-36 - skipped (10 entries)

SEQ ID NO: 37 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 38 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 39 - DNA, 156 bp, synthetic construct
  source 1..156: mol_type = other DNA
tataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacata

SEQ ID NO: 40 - DNA, 12 bp, synthetic construct
  source 1..12: mol_type = other DNA
ccgtggtccttc

SEQ ID NO: 41 - DNA, 22 bp, synthetic construct
  source 1..22: mol_type = other DNA
gtctcctctgacttcaacagcg

SEQ ID NO: 42 - DNA, 22 bp, synthetic construct
  source 1..22: mol_type = other DNA
accaccctgttgctgtagccaa

SEQ ID NO: 43 - DNA, 22 bp, synthetic construct
  source 1..22: mol_type = other DNA
agacagccactcacctcttcag

SEQ ID NO: 44 - DNA, 22 bp, synthetic construct
  source 1..22: mol_type = other DNA
ttctgccagtgcctctttgctg

SEQ ID NO: 45 - DNA, 23 bp, synthetic construct
  source 1..23: mol_type = other DNA
ggtgagaagagatgtctgaatcc

SEQ ID NO: 46 - DNA, 22 bp, synthetic construct
  source 1..22: mol_type = other DNA
gtccatccttggaagcactgca

SEQ ID NO: 47 - DNA, 22 bp, synthetic construct
  source 1..22: mol_type = other DNA
gcctgtcaacagcagtctactg

SEQ ID NO: 48 - DNA, 24 bp, synthetic construct
  source 1..24: mol_type = other DNA
tgtgcttagagaagcaagatactc

SEQ ID NO: 49 - DNA, 22 bp, synthetic construct
  source 1..22: mol_type = other DNA
aactgggaagggctgccacatt

SEQ ID NO: 50 - DNA, 22 bp, synthetic construct
  source 1..22: mol_type = other DNA
ggaagacaggagagctgcaact

SEQ ID NO: 51 - DNA, 24 bp, synthetic construct
  source 1..24: mol_type = other DNA
cttggattcctacaaagaagcagc

SEQ ID NO: 52 - DNA, 22 bp, synthetic construct
  source 1..22: mol_type = other DNA
tcctccttctggaactgctgca

SEQ ID NO: 53 - DNA, 22 bp, synthetic construct
  source 1..22: mol_type = other DNA
agcttgcctcaatcctgcatcc

SEQ ID NO: 54 - DNA, 22 bp, synthetic construct
  source 1..22: mol_type = other DNA
tccttcaggaacagccaccagt

SEQ ID NO: 55 - DNA, 28 bp, synthetic construct
  source 1..28: mol_type = other DNA
tatcagcacacaattgcccattatacgc

SEQ ID NO: 56 - DNA, 28 bp, synthetic construct
  source 1..28: mol_type = other DNA
gcgtataatgggcaattgtgtgctgata

SEQ ID NO: 57 - DNA, 28 bp, synthetic construct
  source 1..28: mol_type = other DNA
tatcagcacacaatagtccattatacgc

SEQ ID NO: 58 - DNA, 28 bp, synthetic construct
  source 1..28: mol_type = other DNA
gcgtataatggactattgtgtgctgata

SEQ ID NO: 59 - DNA, 32 bp, synthetic construct
  source 1..32: mol_type = other DNA
aattctcctccccaccttccccaccctcccca

SEQ ID NO: 60 - DNA, 21 bp, synthetic construct
  source 1..21: mol_type = other DNA
acctatttcagcatactacgc

SEQ ID NO: 61 - DNA, 21 bp, synthetic construct
  source 1..21: mol_type = other DNA
gcgtagtatgctgaaataggt

SEQ ID NO: 62 - DNA, 44 bp, synthetic construct
  source 1..44: mol_type = other DNA
cacacaattgcccattatacgcgcgtataatgggcaattgtgtg

SEQ ID NO: 63 - DNA, 14 bp, synthetic construct
  source 1..14: mol_type = other DNA
taaatataatttaa

SEQ ID NO: 64 - DNA, 14 bp, synthetic construct
  source 1..14: mol_type = other DNA
ttaaattatattta

SEQ ID NO: 65 - DNA, 14 bp, synthetic construct
  source 1..14: mol_type = other DNA
aatatataatctaa

SEQ ID NO: 66 - DNA, 14 bp, synthetic construct
  source 1..14: mol_type = other DNA
ttagattatatatt

SEQ ID NO: 67 - DNA, 14 bp, synthetic construct
  source 1..14: mol_type = other DNA
tatttattatcttt

SEQ ID NO: 68 - DNA, 14 bp, synthetic construct
  source 1..14: mol_type = other DNA
aaagataataaata

SEQ ID NO: 69 - DNA, 32 bp, synthetic construct
  source 1..32: mol_type = other DNA
atataatttttaattagtatagaatatgttaa

SEQ ID NO: 70 - DNA, 32 bp, synthetic construct
  source 1..32: mol_type = other DNA
ttaacatactctatactaattaaaaattatat

SEQ ID NO: 71 - DNA, 26 bp, synthetic construct
  source 1..26: mol_type = other DNA
tataatttgatattagtacaaatccc

SEQ ID NO: 72 - DNA, 26 bp, synthetic construct
  source 1..26: mol_type = other DNA
gggatttgtactaatatcaaattata

SEQ ID NO: 73 - DNA, 27 bp, synthetic construct
  source 1..27: mol_type = other DNA
atataatatttatttagtacaaagttc

SEQ ID NO: 74 - DNA, 27 bp, synthetic construct
  source 1..27: mol_type = other DNA
gaactttgtactaaataaatattatat

SEQ ID NO: 75 - DNA, 27 bp, synthetic construct
  source 1..27: mol_type = other DNA
atataattttttattagtatagagtat

SEQ ID NO: 76 - DNA, 27 bp, synthetic construct
  source 1..27: mol_type = other DNA
atactctatactaataaaaaattatat

SEQ ID NO: 77 - DNA, 16 bp, synthetic construct
  source 1..16: mol_type = other DNA
tagtataaaaaactgt

SEQ ID NO: 78 - DNA, 16 bp, synthetic construct
  source 1..16: mol_type = other DNA
acagttttttatacta

SEQ ID NO: 79 - DNA, 16 bp, synthetic construct
  source 1..16: mol_type = other DNA
tagtatacaaaagatt

SEQ ID NO: 80 - DNA, 16 bp, synthetic construct
  source 1..16: mol_type = other DNA
aatcttttgtatacta

SEQ ID NO: 81 - DNA, 16 bp, synthetic construct
  source 1..16: mol_type = other DNA
tagtatatatatctct

SEQ ID NO: 82 - DNA, 16 bp, synthetic construct
  source 1..16: mol_type = other DNA
agagatatatatacta

SEQ ID NO: 83 - DNA, 16 bp, synthetic construct
  source 1..16: mol_type = other DNA
tagtataaaaaaaatt

SEQ ID NO: 84 - DNA, 16 bp, synthetic construct
  source 1..16: mol_type = other DNA
aattttttttatacta

SEQ ID NO: 85 - DNA, 47 bp, synthetic construct
  source 1..47: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 11: mod_base = OTHER; note = Uracil
  modified_base 42: mod_base = OTHER; note = Uracil
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
cccgaggcggtacgagccacacgtactacgctcgtaccgcctcgggt

SEQ ID NO: 86 - DNA, 48 bp, synthetic construct
  source 1..48: mol_type = other DNA
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 12: mod_base = OTHER; note = Uracil
  modified_base 43: mod_base = OTHER; note = Uracil
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage
acccgaggcggtacgagccacacgtactacgctcgtaccgcctcgggt

SEQ ID NO: 87 - DNA, 47 bp, synthetic construct
  source 1..47: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 12: mod_base = OTHER; note = Uracil
  modified_base 39: mod_base = OTHER; note = Uracil
  modified_base 40: mod_base = OTHER; note = Nucleotide with phosphorothioate linkage
  modified_base 41: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
cccgagcaggatcgagccacacgtactacgctcgatcctgctcgggt

SEQ ID NO: 88 - DNA, 48 bp, synthetic construct
  source 1..48: mol_type = other DNA
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 13: mod_base = OTHER; note = Uracil
  modified_base 40: mod_base = OTHER; note = Uracil
  modified_base 41: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage
acccgagcaggatcgagccacacgtactacgctcgatcctgctcgggt

SEQ ID NO: 89 - DNA, 34 bp, synthetic construct
  source 1..34: mol_type = other DNA
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 17: mod_base = OTHER; note = Uracil
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
cccgggcggacagcactcacgactccgcccgggt

SEQ ID NO: 90 - DNA, 35 bp, synthetic construct
  source 1..35: mol_type = other DNA
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 18: mod_base = OTHER; note = Uracil
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
  modified_base 24: mod_base = OTHER; note = Nucleotide with phosphorothioate linkage
acccgggcggacagcactcacgactccgcccgggt

SEQ ID NO: 91 - DNA, 17 bp, synthetic construct
  source 1..17: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
cacgactccgcccgggt

SEQ ID NO: 92 - DNA, 17 bp, synthetic construct
  source 1..17: mol_type = other DNA
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
acccgggcggacagcac

SEQ ID NO: 93 - DNA, 43 bp, synthetic construct
  source 1..43: mol_type = other DNA
cccgggcggaagagccacacgtactacgctcttccgcccgggt

SEQ ID NO: 94 - DNA, 21 bp, synthetic construct
  source 1..21: mol_type = other DNA
cccgggcggatccgcccgggt

SEQ ID NO: 95 - DNA, 53 bp, synthetic construct
  source 1..53: mol_type = other DNA
cccgggcggaaattctcctccccaccttccccaccctccccatccgcccgggt

SEQ ID NO: 96 - DNA, 16 bp, synthetic construct
  source 1..16: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
cacgactccgcccggg

SEQ ID NO: 97 - DNA, 16 bp, synthetic construct
  source 1..16: mol_type = other DNA
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
cccgggcggacagcac

SEQ ID NO: 98 - DNA, 47 bp, synthetic construct; identical to SEQ ID NO: 87
  source 1..47: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 40: mod_base = OTHER; note = Nucleotide with phosphorothioate linkage
  modified_base 41: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage

SEQ ID NO: 99 - DNA, 48 bp, synthetic construct; identical to SEQ ID NO: 88
  source 1..48: mol_type = other DNA
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 41: mod_base = OTHER; note = Nucleotide with phosphorothioate linkage
  modified_base 42: mod_base = OTHER; note = Nucleotide with phosphorothioate linkage
  modified_base 43: mod_base = OTHER; note = Nucleotide with phosphorothioate linkage
  modified_base 44: mod_base = OTHER; note = Nucleotide with phosphorothioate linkage
  modified_base 45: mod_base = OTHER; note = Nucleotide with phosphorothioate linkage
  modified_base 46: mod_base = OTHER; note = Nucleotide with phosphorothioate linkage

SEQ ID NO: 100 - DNA, 56 bp, synthetic construct
  source 1..56: mol_type = other DNA
tatcagcacacaattgcccattatacgcgcgtataatggactattgtgtgctgata

SEQ ID NO: 101 - DNA, 56 bp, synthetic construct
  source 1..56: mol_type = other DNA
tatcagcacacaatagtccattatacgcgcgtataatggactattgtgtgctgata

SEQ ID NO: 102 - DNA, 18 bp, synthetic construct
  source 1..18: mol_type = other DNA
ccgtggtccttcggtacc

SEQ ID NO: 103 - DNA, 18 bp, synthetic construct
  source 1..18: mol_type = other DNA
ggtaccgccttccaccgg

SEQ ID NO: 104 - DNA, 18 bp, synthetic construct
  source 1..18: mol_type = other DNA
ggtaccgaaggaccacgg

SEQ ID NO: 105 - DNA, 18 bp, synthetic construct
  source 1..18: mol_type = other DNA
ccggtggaaggcggtacc

SEQ ID NO: 106 - DNA, 46 bp, unidentified
  source 1..46: mol_type = unassigned DNA
ctggggactttccagcctggggactttccagctgggactttccagg

SEQ ID NO: 107 - DNA, 72 bp, Homo sapiens
  source 1..72: mol_type = genomic DNA
ggtgtggaaagtccccaggctccccagcaggcagaagtatgcaaagcatgcatctcaattagtcagcaacca

SEQ ID NO: 108 - DNA, 336 bp, Homo sapiens
  source 1..336: mol_type = genomic DNA
cagggcagcaggggcaggtgccagcaaggaaggcaggcacgccaggaagacacccatggtgagaagtgcagatggcccgagggcaagtttgctcaactcacccaggtttgctcttgctggggccaagaggactcatgtgccagggccaagggcccttgggggctctcacagggggcttatctgggcttcggttctggagggccaggaacaaacaggcttcaaagccaagggcttggctggcacacagggggcttggtccttcacctctgtcccctctccctacggacacatataagaccctggtcacacctgggagaggaggagaggagagcatag

SEQ ID NO: 109 - DNA, 36 bp, Homo sapiens
  source 1..36: mol_type = genomic DNA
ggctggtcttgaactcctgrgctcargtgatcctcc

SEQ ID NO: 110 - DNA, 27 bp, Homo sapiens
  source 1..27: mol_type = genomic DNA
tgcggccaaatctcccgccaggtcagc

SEQ ID NO: 111 - DNA, 27 bp, Homo sapiens
  source 1..27: mol_type = genomic DNA
gctgacctggcgggagatttggccgca

SEQ ID NO: 112 - DNA, 32 bp, Homo sapiens; identical to SEQ ID NO: 59
  source 1..32: mol_type = genomic DNA

SEQ ID NO: 113 - DNA, 18 bp, Homo sapiens
  source 1..18: mol_type = genomic DNA
  misc_difference 2: note = a, c, t, g, unknown or other
  misc_difference 4: note = a, c, t, g, unknown or other
  misc_difference 5: note = a, c, t, g, unknown or other
  misc_difference 7: note = a, c, t, g, unknown or other
  misc_difference 8: note = a, c, t, g, unknown or other
  misc_difference 11: note = a, c, t, g, unknown or other
  misc_difference 15: note = a, c, t, g, unknown or other
  misc_difference 17: note = a, c, t, g, unknown or other
rnynncnngyngktnyny

SEQ ID NO: 114 - DNA, 11 bp, Homo sapiens
  source 1..11: mol_type = genomic DNA
wtttatrtttw

SEQ ID NO: 115 - DNA, 404 bp, Homo sapiens
  source 1..404: mol_type = genomic DNA
aggcagacccaggggccgcatgcagcagggcctgaggagggaggtgtggacggaggaggcccgctgccattcttggtatggttctcactccaggagcacagctgcatctggtctcactctgggcagcttataaggcctggtgtgagttttgtttatgcaagtgcagcataaaaggaacaaatctaccagcaccggggctgttgccactgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagac

SEQ ID NO: 116 - DNA, 46 bp, Homo sapiens; identical to SEQ ID NO: 106
  source 1..46: mol_type = genomic DNA

SEQ ID NO: 117 - DNA, 11 bp, Homo sapiens
  source 1..11: mol_type = genomic DNA
ggggactttcc

SEQ ID NO: 118 - DNA, 55 bp, Homo sapiens
  source 1..55: mol_type = genomic DNA
ggggactttccggggactttccggggactttccggggactttccggggactttcc

SEQ ID NO: 119 - DNA, 176 bp, Homo sapiens
  source 1..176: mol_type = genomic DNA
ttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagac

SEQ ID NO: 120 - DNA, 2186 bp, Homo sapiens
  source 1..2186: mol_type = genomic DNA
catgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgc

SEQ ID NO: 121 - DNA, 156 bp, Homo sapiens; identical to SEQ ID NO: 39
  source 1..156: mol_type = genomic DNA

SEQ ID NO: 122 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 123 - DNA, 24 bp, synthetic construct
  source 1..24: mol_type = other DNA
tgtatgtgactgcccaagatgaag

SEQ ID NO: 124 - DNA, 22 bp, synthetic construct
  source 1..22: mol_type = other DNA
agaggaggttggtctcactacc

SEQ ID NO: 125 - DNA, 23 bp, synthetic construct
  source 1..23: mol_type = other DNA
gagagtgattgagagtggaccac

SEQ ID NO: 126 - DNA, 22 bp, synthetic construct
  source 1..22: mol_type = other DNA
cacaaccctctgcacccagttt

SEQ ID NO: 127 - DNA, 23 bp, synthetic construct
  source 1..23: mol_type = other DNA
agaatcaccagcagcaagtgtcc

SEQ ID NO: 128 - DNA, 22 bp, synthetic construct
  source 1..22: mol_type = other DNA
tcctgaacccacttctgcttgg

SEQ ID NO: 129 - DNA, 22 bp, synthetic construct
  source 1..22: mol_type = other DNA
cctgctgctttgcctacattgc

SEQ ID NO: 130 - DNA, 22 bp, synthetic construct
  source 1..22: mol_type = other DNA
acacacttggcggttctttcgg

SEQ ID NO: 131 - DNA, 22 bp, synthetic construct
  source 1..22: mol_type = other DNA
ggcagaaagcttgtctcaaccc

SEQ ID NO: 132 - DNA, 22 bp, synthetic construct
  source 1..22: mol_type = other DNA
ctccttcaggaacagccaccaa

SEQ ID NO: 133 - DNA, 22 bp, synthetic construct
  source 1..22: mol_type = other DNA
ctcttctgcctgctgcactttg

SEQ ID NO: 134 - DNA, 22 bp, synthetic construct
  source 1..22: mol_type = other DNA
atgggctacaggcttgtcactc

SEQ ID NO: 135 - DNA, 22 bp, synthetic construct
  source 1..22: mol_type = other DNA
gagccttctctgctccctgata

SEQ ID NO: 136 - DNA, 22 bp, synthetic construct
  source 1..22: mol_type = other DNA
gactgaggcttggaatctgctg

SEQ ID NO: 137 - DNA, 22 bp, synthetic construct
  source 1..22: mol_type = other DNA
ccacagaccttccaggagaatg

SEQ ID NO: 138 - DNA, 23 bp, synthetic construct
  source 1..23: mol_type = other DNA
gtgcagttcagtgatcgtacagg

SEQ ID NO: 139 - DNA, 21 bp, synthetic construct
  source 1..21: mol_type = other DNA
gccctcagctacgacccagtg

SEQ ID NO: 140 - DNA, 22 bp, synthetic construct
  source 1..22: mol_type = other DNA
agcttctgtggatagcggtcct

SEQ ID NO: 141 - DNA, 22 bp, synthetic construct
  source 1..22: mol_type = other DNA
gctgtggatgtccaacaagagg

SEQ ID NO: 142 - DNA, 22 bp, synthetic construct
  source 1..22: mol_type = other DNA
tcctgcatggtgaaggggttca

SEQ ID NO: 143 - DNA, 21 bp, synthetic construct
  source 1..21: mol_type = other DNA
aaccgccacccagagtcctgt

SEQ ID NO: 144 - DNA, 21 bp, synthetic construct
  source 1..21: mol_type = other DNA
acaggcaacgggcgtggtaca

SEQ ID NO: 145 - DNA, 22 bp, synthetic construct
  source 1..22: mol_type = other DNA
ggaataggcacactggagagtc

SEQ ID NO: 146 - DNA, 23 bp, synthetic construct
  source 1..23: mol_type = other DNA
ctctccgtctttcttctccacac

SEQ ID NO: 147 - DNA, 22 bp, synthetic construct
  source 1..22: mol_type = other DNA
gatagccagcctagaggtatgg

SEQ ID NO: 148 - DNA, 24 bp, synthetic construct
  source 1..24: mol_type = other DNA
ccttgatgttatcaggaggattca